(19) United States
(12) Patent Application Publication
(10) Pub. No.: US 2017/0281622 A1
(43) Pub. Date: Oct. 5, 2017

(54) DERIVATIVES OF MACROCYCLIC N-ARYL-2-AMINO-4-ARYL-PYRIMIDINE POLYETHERS AS INHIBITORS OF FTL3 AND JAK

(71) Applicant: PIERRE FABRE MEDICAMENT, Boulogne-Billancourt (FR)

(72) Inventors: El Bachir KALOUN, Roquettes (FR); Serge GRISONI, Portet Sur Garonne (FR); Bruno GOMES, Saint Lys (FR); Philippe SCHMITT, Nailloux (FR)

(73) Assignee: PIERRE FABRE MEDICAMENT, Boulogne-Billancourt (FR)

(21) Appl. No.: 15/508,218

(22) PCT Filed: Sep. 2, 2015
(86) PCT No.: PCT/EP2015/070066

§ 371 Date: Mar. 2, 2017

(30) Foreign Application Priority Data
Sep. 2, 2014 (FR) 1458203

(51) Int. Cl. A61K 31/505; A61K 31/44; A61K 31/496; C08G 65/00; A61K 31/4412; A61K 31/165

(52) U.S. Cl. CPC A61K 31/505; A61K 31/4412; A61K 31/165; A61K 31/496; C08G 65/00; A61K 31/44

(57) ABSTRACT

The present invention relates to a compound with the following formula: formula (I) or a salt and/or a pharmaceutically acceptable solvate thereof, in particular for use as a drug, in particular in the treatment of cancer, as well as to the pharmaceutical compositions that contain same and to the methods for preparing same.

The present invention relates to derivatives of macrocyclic N-aryl-2-amino-4-aryl-pyrimidine polyethers, and to the therapeutic use thereof, particularly in the treatment of cancer, and to the methods for synthesizing same.

Mutations of tyrosine kinase receptors play a crucial role in the pathogenesis of many cancers. For example, the FLT3 receptor is often mutated in acute myeloid leukemia (in about 30% of cases) (Gilliland et al. 2002 Blood 100: 1532-1542). Mutations that result in an increase in the kinase activity of the FLT3 receptor make the tumor cell relatively dependent on this receptor for its proliferation and survival, which thus makes this mutated receptor a relevant target in oncology. Three types of FLT3-activating mutations are identified today in acute myeloid leukemia (AML): internal tandem duplication (FLT3-ITD), which is detected in about 20% of cases, point mutations in the catalytic domain of the receptor, which constitute 6-8% of cases, and point mutations in the juxtamembrane and extracellular domains, which are rare (2%) (Kayser et al. 2014 Leukemia & Lymphoma 55: 243-255).

The new generations of FLT3 inhibitors undergoing clinical evaluation have shown encouraging results for the treatment of AMLs expressing a mutated form of FLT3. However, most patient responses remain insufficient as they are incomplete and transient, resulting in a relapse rate that remains too high. The causes of these relapses/resistances are many. They can bring into play secondary mutations of the FLT3 receptor or the activation of alternative signaling pathways leading to downstream reactivation of the FLT3 receptor pathway. In addition, whereas the leukemic cells circulating in the patient's blood can be relatively sensitive to the tyrosine kinase inhibitors, the leukemic cells harbored in the patient's marrow are more refractory to treatment, suggesting a role of the bone marrow (microenvironment) in therapeutic resistance (Weisberg et al. 2012 Leukemia 26: 2233-2244). This stromal microenvironment of the leukemic cells, constituted by the bone marrow, would protect the leukemic cells from the effects of tyrosine kinase inhibitors. The IL-6/JAK/STAT signaling pathway is one of the major pathways that would help confer a survival advantage on the leukemic cells expressing a mutated form of FLT3. Moreover, it has been shown that the therapeutic combination of a JAK inhibitor and an FLT3 inhibitor made it possible to increase the effects of FLT3 inhibition and to overcome the resistance induced by the stromal microenvironment (Weisberg et al. op.cit.). Generally, the JAK family of kinases is described as playing an important role in the control of proliferation, cell survival and apoptosis. These JAK kinases are the object of genetic alterations associated with many tumor pathologies, including hematological malignancies.

The present invention has made it possible, surprisingly, to identify compounds having a dual activity as inhibitor of both JAK and FLT3. These compounds further exhibit a remarkable activity.

The present invention relates to derivatives of macrocyclic N-aryl-2-amino-4-aryl-pyrimidine polyethers, and to the therapeutic use thereof, particularly in the treatment of cancer, and to the method for synthesizing same.

The present invention more particularly relates to a compound of the following general formula (I):

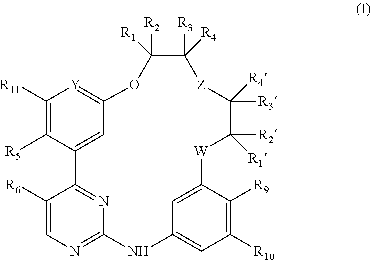

or a pharmaceutically acceptable salt and/or solvate thereof,
wherein:
   W represents an oxygen or sulfur atom,
   Y represents a nitrogen atom or a CRy group wherein Ry represents a hydrogen atom, a halogen atom, a (C₁-C₆)alkyl, (C₁-C₅)haloalkyl, (C₁-C₆)alkoxy, (C₁-C₆)haloalkoxy, OH, CN, NO₂, NR₁₂R₁₃, CO₂H or CO₂((C₁-C₆)alkyl) group,
   Z represents a (CR_{Q1}R_{Q2})ₙQ(CR_{Q3}R_{Q4})ₘ group, wherein n and m represent, independently of each other, an integer between 0 and 3,
   Q represents O, S, S(O) or S(O)₂,
   R_{Q1}, R_{Q2}, R_{Q3} and R_{Q4} represent, independently of each other, a hydrogen atom or a (C₁-C₆)alkyl group,
   R₁, R₂, R₁′ and R₂′ represent, independently of each other, a hydrogen atom or a (C₁-C₆)alkyl group,
   R₃, R₄, R₃′ and R₄′ represent, independently of each other, a hydrogen atom, a (C₁-C₆)alkyl or OH group or R₃ and R₄ and/or R₃′ and R₄′ together form, with the carbon atom that bears them, an optionally substituted monocyclic carbocycle or heterocycle,
   R₅ and R₆ represent, independently of each other, a hydrogen atom, a halogen atom, a (C₁-C₆)alkyl, (C₁-C₆)haloalkyl, (C₁-C₆)alkoxy, (C₁-C₆)haloalkoxy, (C₁-C₆)thioalkoxy, (C₁-C₆)halothioalkoxy, OH, SH, CN, NO₂, or NR₇R₈ group,
   R₉ and R₁₀ represent, independently of each other, a hydrogen atom, a halogen atom, an optionally substituted (C₁-C₆)alkyl, optionally substituted (C₂-C₆)alkenyl, optionally substituted (C₂-C₆)alkynyl, optionally substituted (C₁-C₆)alkoxy, optionally substituted (C₁-C₆)thioalkoxy, CN, NO₂, NR₁₄R₁₅, OH, SH, CO₂R₅₄, CONR₅₅R₅₆ group, an optionally substituted carbocycle or an optionally substituted heterocycle,
   R₁₁ represents a hydrogen atom, a halogen atom, or a (C₁-C₆)alkyl, (C₁-C₆)haloalkyl, (C₁-C₆)alkoxy or (C₁-C₆)haloalkoxy group, and
   R₇, R₈, R₁₂, R₁₃, R₁₄, R₁₅, R₅₄, R₅₅ and R₅₆ represent, independently of each other, a hydrogen atom or an optionally substituted (C₁-C₆)alkyl, optionally substituted (C₂-C₆)alkenyl, or optionally substituted (C₂-C₆)alkynyl group, or R₇ and R₈, R₁₂ and R₁₃, R₁₄ and R₁₅ and/or R₅₅ and R₅₆, independently of each other, form with the nitrogen atom that bears them an optionally substituted nitrogen containing heterocycle.

The stereoisomers of the compounds of general formula (I) also form part of the present invention, as well as the mixtures thereof, in particular in the form of a racemic mixture.

The tautomers of the compounds of general formula (I) also form part of the present invention.

By “stereoisomer” is meant, within the meaning of the present invention, a geometrical isomer (or configurational isomer) or an optical isomer.

Geometrical isomers result from the different position of the substituents on a double bond which can then have a Z or E configuration, also called cis or trans.

Optical isomers result in particular from the different spatial position of the substituents on a carbon atom comprising four different substituents. This carbon atom then constitutes a chiral or asymmetrical center. Optical isomers include diastereoisomers and enantiomers. Optical isomers that are non-superimposable mirror images of each other are called “enantiomers”. Optical isomers that are not superimposable mirror images of each other are called “diastereoisomers”.

A mixture containing equal quantities of two individual enantiomeric forms of opposite chirality is called a “racemic mixture”.

By “tautomer” is meant, within the meaning of the present invention, a constitutional isomer of the compound obtained by prototropy, i.e. by migration of a hydrogen atom and change of location of a double bond. The different tautomers of a compound are generally interconvertible and present in equilibrium in solution, in proportions that can vary according to the solvent used, the temperature or the pH.

In the present invention, by “pharmaceutically acceptable” is meant that which is useful in the preparation of a pharmaceutical composition which is generally safe, nontoxic and neither biologically nor otherwise undesirable and which is acceptable for veterinary as well as human pharmaceutical use.

By “pharmaceutically acceptable salt and/or solvate” of a compound is meant a salt and/or solvate that is pharmaceutically acceptable, as defined herein, and that has the desired pharmacological activity of the parent compound.

The pharmaceutically acceptable salts of the compounds of the present invention comprise the conventional nontoxic salts of the compounds of the invention such as those formed from organic or inorganic acids or from organic or inorganic bases. By way of example, mention may be made of the salts derived from inorganic acids such as hydrochloric, hydrobromic, phosphoric, sulfuric acids, and those derived from organic acids such as acetic, trifluoroacetic, propionic, succinic, fumaric, malic, tartaric, citric, ascorbic, maleic, glutamic, benzoic, salicylic, toluenesulfonic, methanesulfonic, stearic, lactic acids. By way of example, mention may be made of the salts derived from inorganic bases such as sodium hydroxide, potassium hydroxide or calcium hydroxide and the salts derived from organic bases such as lysine or arginine.

These salts can be synthesized from the compounds of the invention containing a base or acid moiety and the corresponding acids or bases according to conventional chemical methods.

The pharmaceutically acceptable solvates of the compounds of the present invention comprise conventional solvates such as those formed during the final step of preparation of the compounds of the invention due to the presence of solvents. By way of example, mention may be made of the solvates due to the presence of water (hydrates) or of ethanol.

The term “halogen” represents a fluorine, chlorine, bromine or iodine.

By “(C₁-C₆)alkyl” group is meant, in the meaning of the present invention, a linear or branched, saturated hydrocarbon chain having 1 to 6, particularly 1 to 4, carbon atoms. By way of example, mention may be made of methyl, ethyl, propyl, isopropyl, butyl, isobutyl, sec-butyl, tert-butyl, pentyl or hexyl groups.

By “(C₂-C₆)alkenyl” group is meant, in the meaning of the present invention, a linear or branched hydrocarbon chain having at least one double bond and having 2 to 6, particularly 2 to 4, carbon atoms. By way of example, mention may be made of ethenyl or allyl groups.

By “(C₂-C₆)alkynyl” group is meant, in the meaning of the present invention, a linear or branched hydrocarbon chain having at least one triple bond and having 2 to 6, particularly 2 to 4, carbon atoms. By way of example, mention may be made of ethynyl or propynyl groups.

By “(C₁-C₆)haloalkyl” is meant, in the meaning of the present invention, a (C₁-C₆)alkyl group, as defined above, wherein one or more hydrogen atoms have been each replaced by a halogen atom as defined above. It may be in particular a CF₃ group.

By “(C₁-C₆)alkoxy” group is meant, in the meaning of the present invention, a (C₁-C₆)alkyl group as defined above, attached to the rest of the molecule via an oxygen atom. By way of example, mention may be made of methoxy, ethoxy, propoxy, isopropoxy, butoxy or tert-butoxy groups.

By “(C₁-C₆)haloalkoxy” is meant, in the meaning of the present invention, a (C₁-C₆)haloalkyl group, as defined above, attached to the rest of the molecule via an oxygen atom. It may be in particular an OCF₃ group.

By “(C₁-C₆)thioalkoxy” group is meant, in the meaning of the present invention, a (C₁-C₆)alkyl group as defined above, attached to the rest of the molecule via a sulfur atom. By way of example, mention may be made of thiomethoxy, thioethoxy, thiopropoxy, thio-isopropoxy, thiobutoxy or thio-ted-butoxy groups.

By “(C₁-C₆)halothioalkoxy” is meant, in the meaning of the present invention, a (C₁-C₆)haloalkyl group, as defined above, attached to the rest of the molecule via a sulfur atom. It may be in particular an SCF₃ group.

By “(C₁-C₆)alkyl-amino” group is meant, in the meaning of the present invention, a (C₁-C₆)alkyl group, as defined above, attached to the rest of the molecule via an NH group. By way of example, mention may be made of methylamino, ethylamino, propylamino or butylamino groups.

By “di((C₁-C₆)alkyl)amino” group is meant, in the meaning of the present invention, a (C₁-C₆)alkyl group, as defined above, attached to the rest of the molecule via an NAlk group with Alk representing a (C₁-C₆)alkyl group as defined above. By way of example, mention may be made of dimethylamino, diethylamino, methylethylamino groups, etc.

By “carbocycle” is meant, in the meaning of the present invention, a saturated, unsaturated or aromatic monocyclic or polycyclic hydrocarbon system comprising 3 to 12 carbon atoms. The polycyclic system comprises at least 2, particularly 2 or 3, fused or bridged rings. Each ring of the monocyclic or polycyclic system comprises advantageously 3 to 8, particularly 4 to 7, in particular 5 or 6, carbon atoms. By way of example, mention may be made of an adamantyl, cyclohexyl, cyclopentyl, cyclobutyl, cyclopropyl, cyclohexenyl, phenyl, naphthyl group.

By “aryl” is meant, in the meaning of the present invention, an aromatic hydrocarbon group having preferably 6 to 10 carbon atoms, and comprising one or more fused rings, such as for example a phenyl or naphthyl group. Advantageously, it is phenyl.

By “aryl-(C₁-C₆)alkyl” is meant, in the meaning of the present invention, an aryl group as defined above, attached to the rest of the molecule via a (C₁-C₆)alkyl chain as defined above. By way of example, mention may be made of the benzyl group.

By “(C₁-C₆)alkyl-aryl” is meant, in the meaning of the present invention, a (C₁-C₆)alkyl group as defined above, attached to the rest of the molecule via an aryl group as defined above. By way of example, mention may be made of the tolyl group (CH₃Ph).

By “heterocycle” is meant, in the meaning of the present invention, a saturated, unsaturated or aromatic monocyclic or bicyclic hydrocarbon group, preferably saturated or unsaturated but non-aromatic, containing 3 to 12 carbon atoms, wherein 1 to 4, particularly 1 or 2, carbon atoms are each replaced, independently of each other, by a heteroatom selected from N, O and S, particularly selected from N and O. The bicyclic group comprises two fused or bridged rings. Each ring of the monocyclic group or of the bicyclic group comprises advantageously 3 to 8, particularly 4 to 7, in particular 5 or 6, carbon atoms or heteroatoms forming the ring. By way of example, mention may be made of azetidine, oxetane, thiooxetane, pyrrolidine, pyrroline, pyrrole, tetrahydrofuran, dihydrofuran, furan, tetrahydrothiophene, dihydrothiophene, thiophene, piperidine, dihydropyridine, tetrahydropyridine, pyridine, pyran, dihydropyran, tetrahydropyran, thiopyran, dihydrothiopyran, tetrahydrothiopyran, morpholine, thiomorpholine, piperazine, homopiperazine (or diazepane), azepine, pyrazine, pyrimidine, pyridazine, perhydropyrrolo[3,4-c]pyrrole, 2,5-diazabicyclo[4.2.0]octane, 2,5-diazabicyclo[2.2.1]heptane and imidazole heterocycles. Preferably, the heterocycle will be non-aromatic and can be in particular an azetidine, oxetane, thiooxetane, pyrrolidine, pyrroline, tetrahydrofuran, dihydrofuran, tetrahydrothiophene, dihydrothiophene, piperidine, dihydropyridine, tetrahydropyridine, pyran, dihydropyran, tetrahydropyran, thiopyran, dihydrothiopyran, tetrahydrothiopyran, morpholine, thiomorpholine, piperazine, homopiperazine (or diazepane), perhydropyrrolo[3,4-c]pyrrole, 2.5-diazabicyclo[4.2.0]octane. 3.8-diazabicyclo[3.2.1]octane and 2,5-diazabicyclo[2.2.1]heptane ring.

By “nitrogen containing heterocycle” is meant, in the meaning of the present invention, a heterocycle as defined above comprising at least one nitrogen atom, in particular non-aromatic, preferably saturated. It can be in particular a monocyclic group or a bicyclic group each ring of which comprises 5 to 7, preferably 5 or 6, members and optionally comprising, in addition to the nitrogen atom, another heteroatom preferably selected from oxygen and nitrogen. It will be in particular a piperidine, optionally bridged piperazine (e.g. piperazine, 2,5-diazabicyclo[4.2.0]octane, 3,8-diazabicyclo[3.2.1]octane or 2,5-diazabicyclo[2.2.1]heptane group; particularly a piperazine, 2,5-diazabicyclo[4.2.0]octane or 2,5-diazabicyclo[2.2.1]heptane), morpholine, perhydropyrrolo[3,4-c]pyrrole, diazepane (e.g. 1,3-diazepane or 1,4-diazepane) or pyrrolidine group.

By “fused” rings is meant, in the meaning of the present invention, two rings attached to each other by two adjacent carbon atoms.

By “bridged” rings is meant, in the meaning of the present invention, two rings attached to each other by two non-adjacent carbon atoms,

By “bridged piperazine” is meant, in the meaning of the present invention, a piperazine ring wherein two non-adjacent carbon atoms are connected by a saturated or unsaturated hydrocarbon chain, preferably saturated, comprising advantageously 1 to 5, particularly 1 to 3, preferably 1 or 2 carbon atoms. It can be in particular a 2,5-diazabicyclo[4.2.0]octane, a 3,8-diazabicyclo[3.2.1]octane or a 2,5-diazabicyclo[2.2.1]heptane.

By “unsaturated” group is meant, in the meaning of the present invention, a group comprising at least one C═C or C≡C bond.

By “unsaturated” ring is meant, in the meaning of the present invention, a ring comprising at least one C═C bond but non-aromatic,

By “optionally substituted” group is meant, in the meaning of the present invention, a group optionally substituted by one or more substituents. This/these substituent(s) may be selected particularly from:
a halogen atom,
a (C₁-C₆)alkyl group optionally substituted by one or more groups selected from a halogen atom, OR₁₆, SR₁₇, NR₁₈R₁₉, a carbocycle and a heterocycle,
oxo (═O), CN, NO₂, OR₂₀, SR₂₁, NR₂₂R₂₃, C(O)R₂₄, CO₂R₂₅, OC(O)R₂₅, S(O)R₂₇, SO₂R₂₈, NR₂₉C(O)R₃₀, C(O)NR₃₁R₃₂, NR₃₃CO₂R₃₄, OC(O)NR₃₃R₃₆, NR37CONR₃₈R₃₉ and OCO₂R₄₀ groups,
a carbocycle optionally substituted by one or more groups selected from a halogen atom, a (C₁-C₆)alkyl group, oxo (═O), OR₄₁, SR₄₂ and NR₄₃R₄₄,
a heterocycle optionally substituted by one or more groups selected from a halogen atom, a (C₁-C₆)alkyl group, oxo (═O), OR₄₅, SR₄₆ and NR₄₇R₄₈, and
an —O(CH₂)ₙO— group wherein n represents an integer between 1 and 5, particularly between 2 and 3 (the two oxygens of this group can be attached to the same atom or to two different atoms, advantageously they are attached to the same atom, in particular to the same carbon atom, making it possible in this case to form a cyclic acetal),
   wherein:
R₁₆ to R₄₈ represent, independently of each other, a hydrogen atom, a (C₁-C₆)alkyl, aryl, aryl-(C₁-C₆)alkyl, heterocycle or heterocycle-(C₁-C₆)alkyl group,
   the aryl ring of these groups being optionally substituted by one or more groups selected from a halogen atom and a (C₁-C₆)alkyl group, and
   the heterocyclic ring of these groups being optionally substituted by one or more groups selected from a halogen atom, a (C₁-C₆)alkyl group, and oxo (═O), or
R₂₂ and R₂₃, R₃₁ and R₃₂, R₃₅ and R₃₆, R₃₈ and R₃₉, R₄₃ and R₄₄, and/or R₄₇ and R₄₈ together form, with the nitrogen atom that bears them, a nitrogen containing heterocycle optionally substituted by one or more groups selected from a halogen atom, a (C₁-C₆)alkyl group, and oxo (═O).

Y will represent more particularly a nitrogen atom or a CRy group wherein Ry represents a hydrogen atom, a halogen atom, a (C₁-C₆)alkyl, (C₁-C₆)haloalkyl, (C₁-C₆)alkoxy, (C₁-C₆)haloalkoxy, NR₁₂R₁₃, CO₂H or CO₂((C₁-C₆)alkyl) group, wherein R₁₂ and R₁₃ represent, independently of each other, a hydrogen atom or a (C₁-C₆)alkyl group optionally substituted by one or more halogen atoms, or R₁₂ and R₁₃ form with the nitrogen atom that bears them a preferably non-aromatic 5- or 6-membered heterocycle, optionally comprising another heteroatom selected from O, N and S, and particularly O and N, said heterocycle being optionally substituted by a (C₁-C₆)alkyl group.

Y will represent more particularly a nitrogen atom or a CRy group wherein Ry represents a hydrogen atom, a halogen atom, a (C₁-C₆)alkyl, (C₁-C₆)haloalkyl, (C₁-C₆)alkoxy, (C₁-C₆)haloalkoxy or NR₁₂R₁₃ group, wherein R₁₂ and R₁₃ represent, independently of each other, a hydrogen atom or a (C₁-C₆)alkyl group optionally substituted by one or more halogen atoms, or R₁₂ and R₁₃ form with the nitrogen atom that bears them a preferably non-aromatic 5- or 6-membered heterocycle, optionally comprising another heteroatom selected from O, N and S, and particularly O and N, said heterocycle being optionally substituted by a (C₁-C₆)alkyl group.

Y will represent more particularly a nitrogen atom or a CRy group wherein Ry represents a hydrogen atom, a halogen atom, a (C₁-C₆)alkyl, (C₁-C₆)alkoxy, or NR₁₂R₁₃ group, wherein R₁₂ and R₁₃ represent, independently of each other, a hydrogen atom or a (C₁-C₆)alkyl group.

Y will represent in particular a CRy group wherein Ry is as defined according to one of the previous definitions. Ry will represent in particular a hydrogen atom, a halogen atom, a (C₁-C₆)alkyl or (C₁-C₆)haloalkyl group; advantageously a hydrogen atom or a halogen atom (e.g. F). Y will represent in particular a CH or CF group.

Q will represent more particularly an oxygen atom.

n and m represent, independently of each other, 0, 1, 2 or 3. Advantageously, n and m represent, independently of each other, 0 or 1. Preferably, n and m each represent 0.

R₃, R₄, R₃′ and R₄′ represent in particular, independently of each other, a hydrogen atom, a (C₁-C₆)alkyl or OH group, preferably a hydrogen atom or a (C₁-C₆)alkyl group, or R₃ and R₄ and/or R₃′ and R₄′ together form, with the carbon atom that bears them, a monocyclic carbocycle or heterocycle optionally substituted by one or more groups selected from OH, (C₁-C₆)alkyl and oxo (═O).

The monocyclic carbocycle can be in particular a C₃ to C₆, particularly C₅ or C₆, monocyclic carbocycle, particularly saturated, for example a cyclopropyl, a cyclobutyl, a cyclopentyl or a cyclohexyl.

The monocyclic heterocycle can be in particular a monocyclic 3- to 6-membered, particularly a 5- or 6-membered heterocycle, preferably non-aromatic, comprising advantageously an oxygen atom, for example an oxirane, an oxetane, a tetrahydrofuran or a tetrahydropyran.

R₁, R₂, R₁′, R₂′, R₃, R₄, R₃′ and R₄′ will represent more particularly a hydrogen atom or a (C₁-C₆)alkyl group, preferably a hydrogen atom.

According to a particular embodiment, Z═O and R₁═R₂═R₁′═R₂′═R₃═R₄═R₃′═R₄′═H.

R₇, R₈, R₁₂ and R₁₃ represent advantageously, independently of each other, a hydrogen atom or a (C₁-C₆)alkyl, (C₂-C₆)alkenyl or (C₂-C₆)alkynyl group, said group being optionally substituted by one or more groups selected from a halogen atom, CN, OR₅₇, NR₅₈R₅₉, COOR₆₀ and CONR₆₁R₆₂ wherein R₅₇ to R₆₂ represent, independently of each other, a hydrogen atom or a (C₁-C₈)alkyl group,
or R₇ and R₈ and/or R₁₂ and R₁₃, independently of each other, form with the nitrogen atom that bears them a nitrogen containing heterocycle (such as piperidine, optionally bridged piperazine (e.g. piperazine, 2,5-diazabicyclo[4.2.0]octane or 2,5-diazabicyclo[2.2.1]heptane), morpholine, perhydropyrrolo[3,4-c]pyrrole, diazepane (e.g. 1,3-diazepane or 1,4-diazepane) or pyrrolidine) optionally substituted by one or more groups selected from (C₁-C₆)alkyl, (C₂-C₆)alkenyl, (C₂-C₆)alkynyl and C₃ to C₈ saturated or unsaturated monocyclic carbocycle groups.

R₇, R₈, R₁₂ and R₁₃ represent in particular, independently of each other, a hydrogen atom or a (C₁-C₆)alkyl group optionally substituted by one or more groups selected from a halogen atom, CN, OR₅₇, NR₅₈R₅₉, COOR₆₀ and CONR₆₁R₆₂ wherein R₅₇ to R₆₂ represent, independently of each other, a hydrogen atom or a (C₁-C₆)alkyl group,
or R₇ and R₈ and/or R₁₂ and R₁₃, independently of each other, form with the nitrogen atom that bears them a nitrogen containing heterocycle selected from piperidine, piperazine, morpholine and pyrrolidine rings and optionally substituted by one or more (C₁-C₆)alkyl groups.

R₇, R₈, R₁₂ and R₁₃ represent in particular, independently of each other, a hydrogen atom or a (C₁-C₆)alkyl group optionally substituted by one or more groups selected from a halogen atom,
or R₇ and R₈ and/or R₁₂ and R₁₃, independently of each other, form with the nitrogen atom that bears them a nitrogen containing heterocycle selected from piperidine, piperazine, morpholine and pyrrolidine rings and optionally substituted by one or more (C₁-C₆)alkyl groups.

R₇, R₈, R₁₂ and R₁₃ will represent in particular, independently of each other, a hydrogen atom or a (C₁-C₆)alkyl group.

R₅ and R₆ represent, independently of each other, a hydrogen atom, a halogen atom, a (C₁-C₆)alkyl, (C₁-C₆)haloalkyl, (C₁-C₆)alkoxy, (C₁-C₆)haloalkoxy, (C₁-C₆)thioalkoxy, (C₁-C₆)halothioalkoxy, OH, SH, CN, NO₂, or NR₇R₈ group, wherein R₇ and R₈ advantageously represent, independently of each other, a hydrogen atom or a (C₁-C₆)alkyl group optionally substituted by one or more halogen atoms.

R₅ and R₆ represent in particular, independently of each other, a hydrogen atom, a halogen atom, a (C₁-C₆)alkyl, (C₁-C₆)haloalkyl, (C₁-C₆)alkoxy or NR₇R₈ group, wherein R₇ and R₈ advantageously represent, independently of each other, a hydrogen atom or a (C₁-C₆)alkyl group.

R₅ and R₆ represent in particular, independently of each other, a hydrogen atom, a halogen atom, a (C₁-C₆)alkyl or (C₁-C₆)haloalkyl group.

R₅ and R₆ will represent in particular, independently of each other, a hydrogen atom or a (C₁-C₆)alkyl group.

R₁₁ represents advantageously a hydrogen atom, a halogen atom, or a (C₁-C₆)alkyl or (C₁-C₆)haloalkyl group; particularly a hydrogen or halogen atom. R₁₁ can represent in particular H or F.

R₉ and R₁₀ represent, independently of each other, a hydrogen atom, a halogen atom, an optionally substituted (C₁-C₆)alkyl, optionally substituted (C₂-C₆)alkenyl, optionally substituted (C₂-C₆)alkynyl, optionally substituted (C₁-C₅)alkoxy, optionally substituted (C₁-C₆)thioalkoxy, CN, NO₂, OH, SH, NR₁₄R₁₅, CO₂R₅₄, CONR₅₅R₅₆ group, an optionally substituted carbocycle or an optionally substituted heterocycle.

R₉ and R₁₀ represent more particularly, independently of each other, a hydrogen atom, a halogen atom, an optionally substituted (C₁-C₆)alkyl, optionally substituted (C₂-C₆)alkenyl, optionally substituted (C₂-C₆)alkynyl, optionally substituted (C₁-C₆)alkoxy, optionally substituted (C₁-C₆)thioalkoxy, NR₁₄R₁₅, CO₂R₅₄, CONR₅₅R₅₆ group, an optionally substituted carbocycle or an optionally substituted heterocycle, notably wherein R₁₅≠H and R₅₆≠H.

R₉ and R₁₀ represent in particular, independently of each other, a hydrogen atom, a halogen atom, an optionally substituted (C₁-C₆)alkyl, optionally substituted (C₂-C₆)alkenyl, optionally substituted (C₂-C₆)alkynyl, optionally substituted (C₁-C₆)alkoxy, optionally substituted (C₁-C₆)thioalkoxy, NR₁₄R₁₅, CO₂R₅₄, CONR₅₅R₅₆ group, or an optionally substituted heterocycle, notably wherein R₁₅≠H and R₅₆≠H.

R₉ and R₁₀ represent in particular, independently of each other, a hydrogen atom, a halogen atom, CO₂R₅₄, CONR₅₅R₅₆, or a (C₁-C₆)alkyl, (C₂-C₆)alkenyl, (C₂-C₆)alkynyl, (C₁-C₆)alkoxy, (C₁-C₆)thioalkoxy, (C₁-C₆)alkyl-amino, di((C₁-C₆)alkyl)amino or heterocycle group, said group being optionally substituted, notably wherein R₅₆≠H and preferably wherein R₅₅ and R₅₆ form with the nitrogen atom that bears them an optionally substituted nitrogen containing heterocycle.

R₉ and R₁₀ represent more particularly, independently of each other, a hydrogen atom, a halogen atom, CONR₅₅R₅₆, or a (C₁-C₆)alkyl, (C₂-C₆)alkenyl, (C₂-C₆)alkynyl, (C₁-C₆)alkoxy, (C₁-C₆)thioalkoxy, (C₁-C₆)alkyl-amino, di((C₁-C₆)alkyl)amino or heterocycle group, said group being optionally substituted, notably wherein R₅₆≠H and preferably wherein R₅₅ and R₅₆ form with the nitrogen atom that bears them an optionally substituted nitrogen containing heterocycle.

In the preceding definitions of R₉ and R₁₀, R₁₄ and R₁₅ represent, independently of each other, a hydrogen atom or an optionally substituted (C₁-C₆)alkyl, optionally substituted (C₂-C₆)alkenyl, or optionally substituted (C₂-C₆)alkynyl group, or R₁₄ and R₁₅ form with the nitrogen atom that bears them an optionally substituted nitrogen containing heterocycle. In particular, R₁₄ can represent a hydrogen atom or a (C₁-C₆)alkyl group and R₁₅ can represent a hydrogen atom or an optionally substituted (C₁-C₆)alkyl, optionally substituted (C₂-C₆)alkenyl, or optionally substituted (C₂-C₆)alkynyl group, or R₁₄ and R₁₅ will form with the nitrogen atom that bears them an optionally substituted nitrogen containing heterocycle. Advantageously, R₁₄ will represent a hydrogen atom or a (C₁-C₆)alkyl group and R₁₅ will represent a hydrogen atom or an optionally substituted (C₁-C₆)alkyl group, or R₁₄ and R₁₅ will form with the nitrogen atom that bears them an optionally substituted nitrogen containing heterocycle. Preferably, the optionally substituted nitrogen containing heterocycle will be a monocyclic or bicyclic nitrogen containing heterocycle, preferably monocyclic, particularly non-aromatic, preferably saturated, each ring of which comprises 5 to 7 members, optionally comprising 1 heteroatom in addition to the nitrogen atom selected from N and O, such as an optionally bridged piperazine, piperidine, morpholine, perhydropyrrolo[3,4-c]pyrrole, diazepane (e.g. 1,3-diazepane or 1,4-diazepane) or pyrrolidine ring, the heterocycle being optionally substituted by one or more groups selected from a halogen atom, a (C₁-C₆)alkyl group and oxo (═O).

Preferentially, the optionally bridged piperazine will be a piperazine, 2,5-diazabicyclo[4.2.0]octane, 3,8-diazabicyclo[3.2.1]octane or 2,5-diazabicyclo[2.2.1]heptane ring, particularly a piperazine, 2,5-diazabicyclo[4.2.0]octane or 2,5-diazabicyclo[2.2.1]heptane ring.

In the preceding definitions of R₉ and R₁₀, a carbocycle is more particularly a C₃ to C₆, particularly C₅ or C₆, monocyclic carbocycle, particularly saturated, for example a cyclopropyl, a cyclobutyl, a cyclopentyl, a cyclohexyl or a cyclohexenyl.

In the preceding definitions of R₉ and R₁₀, a heterocycle is more particularly a monocyclic or bicyclic heterocycle, preferably monocyclic, each ring having 5, 6 or 7 members, particularly 6 or 7 members, saturated, unsaturated or aromatic, particularly saturated or aromatic, preferably saturated, the heterocycle comprising 1 or 2 heteroatoms selected from N, O and S, particularly from N and O, and preferably comprising at least one nitrogen atom. The heterocycle can be for example a pyrrolidine, pyrroline, pyrrole, tetrahydrofuran, dihydrofuran, furan, tetrahydrothiophene, dihydrothiophene, thiophene, piperidine, dihydropyridine, tetrahydropyridine, pyridine, pyran, dihydropyran, tetrahydropyran, thiopyran, dihydrothiopyran, tetrahydrothiopyran, morpholine, thiomorpholine, piperazine, homopiperazine, azepine, pyrazine, pyrimidine, pyridazine, perhydropyrrolo[3,4-c]pyrrole, 2,5-diazabicyclo[4.2.0]octane, 2,5-diazabicyclo[2.2.1]heptane, 3,8-diazabicyclo[3.2.1]octane or imidazole ring. The heterocycle will be in particular a pyrrolidine, pyrroline, pyrrole, tetrahydrofuran, dihydrofuran, furan, piperidine, dihydropyridine, tetrahydropyridine, pyridine, pyran, dihydropyran, tetrahydropyran, morpholine, piperazine, homopiperazine, azepine, pyrazine, pyrimidine, pyridazine, perhydropyrrolo[3,4-c]pyrrole, 2,5-diazabicyclo[4.2.0]octane, 2,5-diazabicyclo[2.2.1]heptane, 3,8-diazabicyclo[3.2.1]octane or imidazole ring. The heterocycle will be in particular a pyrrolidine, pyrroline, pyrrole, piperidine, dihydropyridine, tetrahydropyridine, pyridine, morpholine, piperazine, homopiperazine, azepine, pyrazine, pyrimidine, pyridazine, perhydropyrrolo[3,4-c]pyrrole, 2,5-diazabicyclo[4.2.0]octane, 2,5-diazabicyclo[2.2.1]heptane, 3,8-diazabicyclo[3.2.1]octane or imidazole ring. The heterocycle will be more particularly a pyrrolidine, pyrroline, pyrrole, piperidine, dihydropyridine, tetrahydropyridine, pyridine, morpholine, piperazine, homopiperazine, azepine, pyrazine, pyrimidine, pyridazine or imidazole ring. The heterocycle will be in particular a pyrrolidine, pyrrole, piperidine, pyridine, morpholine, piperazine, homopiperazine (e.g. 1,3-diazepane or 1,4-diazepane), azepine, pyrazine, pyrimidine, pyridazine or imidazole ring. The heterocycle can be advantageously a piperidine, pyridine, morpholine, piperazine or 1,4-diazepane ring.

In the preceding definitions of R₉, R₁₀, R₁₄ and R₁₅, an optionally substituted group or ring is a group or ring optionally substituted by one or more substituents, advantageously selected from:
a halogen atom,
a (C₁-C₆)alkyl group optionally substituted by one or more groups selected from a halogen atom, OR₁₆, SR₁₇, NR₁₈R₁₉, a carbocycle and a heterocycle,
oxo (═O), CN, NO₂, OR₂₀, SR₂₁, NR₂₂R₂₃, C(O)R₂₄, CO₂R₂₅, OC(O)R₂₅, S(O)R₂₇, SO₂R₂₈, NR₂₉C(O)R₃₀, C(O)NR₃₁R₃₂, NR₃₃CO₂R₃₄, OC(O)NR₃₅R₃₆, NR₃₇CONR₃₈R₃₉ and OCO₂R₄₀ groups,
a carbocycle optionally substituted by one or more groups selected from a halogen atom, a (C₁-C₆)alkyl group, oxo (═O), OR₄₁, SR₄₂ and NR₄₃R₄₄,
a heterocycle optionally substituted by one or more groups selected from a halogen atom, a (C₁-C₆)alkyl group, oxo (═O), OR₄₅, SR₄₆ and NR₄₇R₄₈, and
an —O(CH₂)ₙO— group wherein n represents an integer between 1 and 5, particularly between 2 and 3 (the two oxygens of this group can be attached to the same atom or to two different atoms, advantageously they are attached to the same atom, in particular to the same carbon atom, making it possible in this case to form a cyclic acetal),
   wherein:
R₁₆ to R₄₈ represent, independently of each other, a hydrogen atom, a (C₁-C₆)alkyl, aryl, aryl-(C₁-C₆)alkyl, heterocycle or heterocycle-(C₁-C₆)alkyl group,
   the aryl ring of these groups being optionally substituted by one or more groups selected from a halogen atom and a (C₁-C₆)alkyl group, and
   the heterocyclic ring of these groups being optionally substituted by one or more groups selected from a halogen atom, a (C₁-C₆)alkyl group, and oxo (═O), or
R₂₂ and R₂₃, R₃₁ and R₃₂, R₃₅ and R₃₆, R₃₈ and R₃₉, R₄₃ and R₄₄, and/or R₄₇ and R₄₈ together form, with the nitrogen atom that bears them, a nitrogen containing heterocycle optionally substituted by one or more groups selected from a halogen atom, a (C₁-C₆)alkyl group, and oxo (═O).

In the preceding definitions of R₉, R₁₀, R₁₄ and R₁₅, the optionally substituted groups or rings are in particular optionally substituted by one or more substituents selected from:
a halogen atom,
a (C₁-C₆)alkyl group optionally substituted by one or more groups selected from a halogen atom, OR₁₆, NR₁₈R₁₉, a C₃ to C₆ monocyclic carbocycle (particularly saturated) and a 3- to 6-membered monocyclic heterocycle (particularly saturated), oxo (═O), OR₂₀, NR₂₂R₂₃, C(O)R₂₄, CO₂R₂₅, OC(O)R₂₆, NR₂₉C(O)R₃₀, C(O)NR₃₁R₃₂, NR₃₃CO₂R₃₄, OC(O)NR₃₅R₃₆, NR₃₇CONR₃₈R₃₉ and OCO₂R₄₀ groups, and more particularly oxo (═O), OR₂₀, NR₂₂R₂₃, CO₂R₂₅, C(O)NR₃₁R₃₂, NR₃₃CO₂R₃₄, OC(O)NR₃₅R₃₆, NR₃₇CONR₃₈R₃₉ and OCO₂R₄₀ groups,
a C₃ to C₆ carbocycle optionally substituted by one or more groups selected from a halogen atom, a (C₁-C₅)alkyl group, oxo (═O), OR₄₁ and NR₄₃R₄₄,
a 3- to 6-membered, particularly a 5- or 6-membered, heterocycle comprising 1 or 2 heteroatoms selected from N and O, preferably saturated, optionally substituted by one or more groups selected from a halogen atom, a (C₁-C₆)alkyl group, oxo (═O), OR₄₅ and NR₄₇R₄₈, and
an —O(CH₂)ₙO— group wherein n represents an integer equal to 2 or 3 (the two oxygens of this group can be attached to the same atom or to two different atoms, advantageously they are attached to the same atom, in particular to the same carbon atom, making it possible in this case to form a cyclic acetal),
   wherein:
R₁₆, R₁₈ to R₂₀, R₂₂ to R₂₆, R₂₉ to R₄₀, R₄₅ and R₄₇ to R₄₈ represent, independently of each other, a hydrogen atom, a (C₁-C₆)alkyl, aryl, aryl-(C₁-C₆)alkyl, heterocycle or heterocycle-(C₁-C₆)alkyl group, particularly a hydrogen atom, a (C₁-C₆)alkyl, aryl, or aryl-(C₁-C₆)alkyl group, in particular a hydrogen atom, or a (C₁-C₆)alkyl group,
   the aryl ring of these groups being a phenyl group and being optionally substituted by one or more groups selected from a halogen atom and a (C₁-C₆)alkyl group, and
   the heterocyclic ring of these groups being a 3- to 6-membered, particularly a 5- or 6-membered, heterocycle comprising 1 or 2 heteroatoms selected from N and O and being optionally substituted by one or more groups selected from a halogen atom, a (C₁-C₆)alkyl group, and oxo (═O), or
R₂₂ and R₂₃, R₃₁ and R₃₂, R₃₅ and R₃₆, R₃₈ and R₃₉, R₄₃ and R₄₄, and/or R₄₇ and R₄₈ together form, with the nitrogen atom that bears them, a 5- or 6-membered nitrogen containing heterocycle, particularly non-aromatic, preferably saturated, optionally comprising 1 heteroatom in addition to the nitrogen atom selected from N and O, such as a piperazine, piperidine, morpholine or pyrrolidine ring, the heterocycle being optionally substituted by one or more groups selected from a halogen atom, a (C₁-C₆)alkyl group, and oxo (═O).

Groups R₀ and R₁₀ can in particular represent, independently of each other:
a hydrogen or halogen atom,
a CO₂R₅₄ group wherein R₅₄ represents a hydrogen atom or a (C₁-C₆)alkyl group optionally substituted by one or more substituents selected from a halogen atom, OR₂₀, NR₂₂R₂₃, C(O)R₂₄, CO₂R₂₅, OC(O)R₂₆, NR₂₉C(O)R₃₀, C(O)NR₃₁R₃₂, NR₃₃CO₂R₃₄, OC(O)NR₃₅R₃₆, NR₃₇CONR₃₈R₃₉ and OCO₂R₄₀; particularly selected from OR₂₀, NR₂₂R₂₃, CO₂R₂₅, C(O)NR₃₁R₃₂, NR₃₃CO₂R₃₄, OC(O)NR₃₅R₃₆, NR₃₇CONR₃₈R₃₉ and OCO₂R₄₀; particularly selected from NR₂₂R₂₃, NR₃₃CO₂R₃₄ and NR₃₇CONR₃₈R₃₉; wherein R₅₄ represents in particular a hydrogen atom or a (C₁-C₆)alkyl group, particularly a hydrogen atom,
a (C₁-C₆)alkyl, (C₂-C₆)alkenyl, (C₂-C₆)alkynyl, (C₁-C₆)alkoxy, (C₁-C₆)thioalkoxy, (C₁-C₆)alkyl-amino or di((C₁-C₆)alkyl)amino group, said group being optionally substituted by one or more substituents selected from a halogen atom, OR₂₀, NR₂₂R₂₃, C(O)R₂₄, CO₂R₂₅, OC(O)R₂₆, NR₂₉C(O)R₃₀, C(O)NR₃₁R₃₂, NR₃₃CO₂R₃₄, OC(O)NR₃₅R₃₆, NR₃₇CONR₃₈R₃₉ and OCO₂R₄₀; particularly selected from OR₂₀, NR₂₂R₂₃, CO₂R₂₅, C(O)NR₃₁R₃₂, NR₃₃CO₂R₃₄, OC(O)NR₃₅R₃₆, NR₃₇CONR₃₈R₃₉ and OCO₂R₄₀; particularly selected from NR₂₂R₂₃, NR₃₃CO₂R₃₄ and NR₃₇CONR₃₈R₃₉, or
a heterocycle or a CONR₅₅R₅₆ group wherein R₅₅ and R₅₆ form with the nitrogen atom that bears them a heterocycle, the heterocycle having 5, 6 or 7 members, particularly 6 or 7 members, comprising 1 or 2 heteroatoms selected from N and O (and comprising at least one nitrogen atom in the case of NR₅₅R₅₆), preferably saturated or aromatic, particularly saturated, (the heterocycle can be in particular an optionally bridged piperazine (e.g. piperazine, 2,5-diazabicyclo[4.2.0]octane, 3,8-diazabicyclo[3.2.1]octane or 2,5-diazabicyclo[2.2.1]heptane), piperidine, perhydropyrrolo[3,4-c]pyrrole, tetrahydropyridine or pyrrolidine ring; particularly a piperazine, piperidine, morpholine, diazepane (e.g. 1,4-diazepane) or pyridine ring) optionally substituted by one or more substituents selected from:
   a halogen atom,
   a (C₁-C₆)alkyl group optionally substituted by one or more groups selected from a halogen atom, OR₁₆, NR₁₈R₁₉, a C₃ to C₆ monocyclic carbocycle (particularly saturated) and a 3- to 6-membered monocyclic heterocycle (particularly saturated),
   oxo (═O), OR₂₀, NR₂₂R₂₃, C(O)R₂₄, CO₂R₂₅, OC(O)R₂₆, NR₂₉C(O)R₃₀, C(O)NR₃₁R₃₂, NR₃₃CO₂R₃₄, OC(O)NR₃₅R₃₆, NR₃₇CONR₃₈R₃₉ and OCO₂R₄₀ groups; and more particularly oxo (═O), OR₂₀, NR₂₂R₂₃, CO₂R₂₅, C(O)NR₃₁R₃₂, NR₃₃CO₂R₃₄, OC(O)NR₃₅R₃₆, NR₃₇CONR₃₈R₃₉ and OCO₂R₄₀ groups,
   a C₃ to C₆ carbocycle optionally substituted by one or more groups selected from a halogen atom, a (C₁-C₆)alkyl group, oxo (═O), OR₄₁ and NR₄₃R₄₄,
   a 3- to 6-membered heterocycle comprising 1 or 2 heteroatoms selected from N and O, particularly saturated or unsaturated, preferably saturated, optionally substituted by one or more groups selected from a halogen atom, a (C₁-C₆)alkyl group, oxo (═O), OR₄₅ and NR₄₇R₄₈, and
   an —O(CH₂)ₙO— group with n representing an integer equal to 2 or 3 (the two oxygens of this group can be attached to the same atom or to two different atoms, advantageously they are attached to the same atom, in particular to the same carbon atom, making it possible in this case to form a cyclic acetal),
      wherein:
R₁₆, R₁₈ to R₂₀, R₂₂ to R₂₆, R₂₉ to R₄₀, R₄₅ and R₄₇ to R₄₈ represent, independently of each other, a hydrogen atom, a (C₁-C₆)alkyl, aryl, aryl-(C₁-C₆)alkyl, heterocycle or heterocycle-(C₁-C₆)alkyl group, particularly a hydrogen atom, a (C₁-C₆)alkyl, aryl, or aryl-(C₁-C₆)alkyl group, in particular a hydrogen atom, or a (C₁-C₆)alkyl group,
   the aryl ring of these groups being a phenyl group and being optionally substituted by one or more groups selected from a halogen atom and a (C₁-C₆)alkyl group, and
   the heterocyclic ring of these groups being a 3- to 6-membered, particularly a 5- or 6-membered, heterocycle comprising 1 or 2 heteroatoms selected from N and O, and being optionally substituted by one or more groups selected from a halogen atom, a (C₁-C₆)alkyl group and oxo (═O), or
R₂₂ and R₂₃, R₃₁ and R₃₂, R₃₅ and R₃₆, R₃₈ and R₃₉, R₄₃ and R₄₄, and/or R₄₇ and R₄₈ together form, with the nitrogen atom that bears them, a 5- or 6-membered nitrogen containing heterocycle, particularly non-aromatic, preferably saturated, optionally comprising 1 heteroatom in addition to the nitrogen atom selected from N and O, such as a piperazine, piperidine, morpholine or pyrrolidine ring, the heterocycle being optionally substituted by one or more groups selected from a halogen atom, a (C₁-C₆)alkyl group, and oxo (═O).

Groups R₉ and R₁₀ can in particular represent, independently of each other:
a hydrogen or halogen atom,
a CO₂R₅₄ group wherein R₅₄ represents a hydrogen atom or a (C₁-C₆)alkyl group optionally substituted by one or more substituents selected from a halogen atom, OR₂₀, NR₂₂R₂₃, C(O)R₂₄, CO₂R₂₅, OC(O)R₂₆, NR₂₉C(O)R₃₀, C(O)NR₃₁R₃₂, NR₃₃CO₂R₃₄, OC(O)NR₃₅R₃₆, NR₃₇CONR₃₈R₃₉ and OCO₂R₄₀; particularly selected from OR₂₀, NR₂₂R₂₃, CO₂R₂₅, C(O)NR₃₁R₃₂, NR₃₃CO₂R₃₄, OC(O)NR₃₅R₃₆, NR₃₇CONR₃₈R₃₉ and OCO₂R₄₀; particularly selected from NR₂₂R₂₃, NR₃₃CO₂R₃₄ and NR₃₇CONR₃₈R₃₉; in particular wherein R₅₄ represents a hydrogen atom or a (C₁-C₆)alkyl group, particularly a hydrogen atom,
a CONR₅₅R₅₆ group wherein R₅₅ and R₅₆ form with the nitrogen atom that bears them a nitrogen containing heterocycle having 5, 6 or 7 members, comprising 1 or 2 heteroatoms selected from N and O of which at least one is a nitrogen atom, particularly non-aromatic, preferably saturated, (the heterocycle can be in particular a piperazine, piperidine, morpholine, diazepane (e.g. 1,4-diazepane) or pyridine ring) optionally substituted by one or more substituents selected from a halogen atom, (C₁-C₆)alkyl, oxo (═O), OR₂₀, NR₂₂R₂₃, C(O)R₂₄, CO₂R₂₅, OC(O)R₂₆, NR₂₉C(O)R₃₀, C(O)NR₃₁R₃₂, NR₃₃CO₂R₃₄, OC(O)NR₃₅R₃₆, NR₃₇CONR₃₈R₃₉ and OCO₂R₄₀: particularly selected from a halogen atom, (C₁-C₆)alkyl, oxo (═O), OR₂₀, NR₂₂R₂₃, CO₂R₂₅, C(O)NR₃₁R₃₂, NR₃₃CO₂R₃₄, OC(O)NR₃₅R₃₆, NR₃₇CONR₃₈R₃₉ and OCO₂R₄₀; notably selected from a halogen atom, (C₁-C₆)alkyl, oxo (═O), OR₂₀, NR₂₂R₂₃, CO₂R₂₅ and C(O)NR₃₁R₃₂,
a (C₁-C₆)alkyl, (C₂-C₆)alkenyl, (C₂-C₆)alkynyl, (C₁-C₆)alkoxy, (C₁-C₆)thioalkoxy, (C₁-C₆)alkyl-amino or di((C₁-C₆)alkyl)amino group, said group being optionally substituted by one or more substituents selected from a halogen atom, OR₂₀, NR₂₂R₂₃, C(O)R₂₄, CO₂R₂₅, OC(O)R₂₆, NR₂₉C(O)R₃₀, C(O)NR₃₁R₃₂, NR₃₃CO₂R₃₄, OC(O)NR₃₅R₃₆, NR₃₇CONR₃₈R₃₉ and OCO₂R₄₀; particularly selected from OR₂₀, NR₂₂R₂₃, CO₂R₂₅, C(O)NR₃₁R₃₂, NR₃₃CO₂R₃₄, OC(O)NR₃₅R₃₆, NR₃₇CONR₃₈R₃₉ and OCO₂R₄₀; notably selected from NR₂₂R₂₃, NR₃₃CO₂R₃₄ and NR₃₇CONR₃₈R₃₆, or
a heterocycle having 5, 6 or 7 members, particularly 6 or 7 members, comprising 1 or 2 heteroatoms selected from N and O and comprising advantageously at least one nitrogen atom, preferably saturated or aromatic, particularly saturated, (the heterocycle can be in particular an optionally bridged piperazine (e.g. piperazine, 2,5-diazabicyclo[4.2.0]octane, 3,8-diazabicyclo[3.2.1]octane or 2,5-diazabicyclo[2.2.1]heptane), piperidine, perhydropyrrolo[3,4-c]pyrrole, tetrahydropyridine or pyrrolidine ring; particularly a piperazine, piperidine, morpholine, diazepane (e.g. 1,4-diazepane) or pyridine ring) optionally substituted by one or more substituents selected from:
   a halogen atom,
   a (C₁-C₆)alkyl group optionally substituted by one or more groups selected from a halogen atom, OR₁₆, NR₁₈R₁₉, a C₃ to C₆ monocyclic carbocycle (particularly saturated) and a 3- to 6-membered monocyclic heterocycle (particularly saturated) comprising 1 or 2 heteroatoms selected from N and O,
   oxo (═O), OR₂₀, NR₂₂R₂₃, C(O)R₂₄, CO₂R₂₅, OC(O)R₂₆, NR₂₉C(O)R₃₀, C(O)NR₃₁R₃₂, NR₃₃CO₂R₃₄, OC(O)NR₃₅R₃₆, NR₃₇CONR₃₈R₃₉ and OCO₂R₄₀ groups; and more particularly oxo (═O), OR₂₀, NR₂₂R₂₃, CO₂R₂₅, C(O)NR₃₁R₃₂, NR₃₃CO₂R₃₄, OC(O)NR₃₅R₃₆, NR₃₇CONR₃₈R₃₉ and OCO₂R₄₀ groups,
   a C₃ to C₆ carbocycle optionally substituted by one or more groups selected from a halogen atom, a (C₁-C₆)alkyl group, oxo (═O), OR₄₁ and NR₄₃R₄₄,
   a 3- to 6-membered heterocycle comprising 1 or 2 heteroatoms selected from N and O, particularly saturated or unsaturated, preferably saturated, optionally substituted by one or more groups selected from a halogen atom, a (C₁-C₆)alkyl group, oxo (═O), OR₄₅ and NR₄₇R₄₈, and
   an —O(CH₂)ₙO— group wherein n represents an integer equal to 2 or 3 (the two oxygens of this group can be attached to the same atom or to two different atoms, advantageously they are attached to the same atom, in particular to the same carbon atom, making it possible in this case to form a cyclic acetal),
      wherein:
R₁₆, R₁₈ to R₂₀, R₂₂ to R₂₆, R₂₉ to R₄₀, R₄₅ and R₄₇ to R₄₈ represent, independently of each other, a hydrogen atom, a (C₁-C₆)alkyl, aryl, aryl-(C₁-C₆)alkyl, heterocycle or heterocycle-(C₁-C₆)alkyl group, particularly a hydrogen atom, a (C₁-C₆)alkyl, aryl, or aryl-(C₁-C₆)alkyl group, in particular a hydrogen atom, or a (C₁-C₆)alkyl group,
   the aryl ring of these groups being a phenyl group and being optionally substituted by one or more groups selected from a halogen atom and a (C₁-C₆)alkyl group, and
   the heterocyclic ring of these groups being a 3- to 6-membered, particularly a 5- or 6-membered, heterocycle comprising 1 or 2 heteroatoms selected from N and O, and being optionally substituted by one or more groups selected from a halogen atom, a (C₁-C₆)alkyl group and oxo (═O), or
R₂₂ and R₂₃, R₃₁ and R₃₂, R₃₅ and R₃₆, R₃₈ and R₃₉, R₄₃ and R₄₄, and/or R₄₇ and R₄₈ together form, with the nitrogen atom that bears them, a 5- or 6-membered nitrogen containing heterocycle, particularly non-aromatic, preferably saturated, optionally comprising 1 heteroatom in addition to the nitrogen atom selected from N and O, such as a piperazine, piperidine, morpholine or pyrrolidine ring, the heterocycle being optionally substituted by one or more groups selected from a halogen atom, a (C₁-C₆)alkyl group, and oxo (═O).

Groups R₉ and R₁₀ can more particularly represent, independently of each other:
a hydrogen or halogen atom (e.g. Br or I),
a CO₂R₅₄ group wherein R₅₄ represents a hydrogen atom or a (C₁-C₆)alkyl group, particularly a hydrogen atom,
a CONR₅₅R₅₆ group wherein R₅₅ and R₅₆ form with the nitrogen atom that bears them a nitrogen containing heterocycle having 5, 6 or 7 members, comprising 1 or 2 heteroatoms selected from N and O of which at least one is a nitrogen atom, particularly non-aromatic, preferably saturated, (the heterocycle can be in particular a piperazine, piperidine, morpholine, diazepane (e.g. 1,4-diazepane) or pyridine ring) optionally substituted by one or more substituents selected from a halogen atom, (C₁-C₆)alkyl, oxo (═O), OR₂₀, NR₂₂R₂₃, CO₂R₂₅, C(O)NR₃₁R₃₂, NR₃₃CO₂R₃₄, OC(O)NR₃₅R₃₆, NR₃₇CONR₃₈R₃₉ and OCO₂R₄₀; particularly selected from a halogen atom, (C₁-C₆)alkyl, oxo (═O), OR₂₀, NR₂₂R₂₃, CO₂R₂₅ and C(O)NR₃₁R₃₂,
a —Z₁—(CH₂)ₘ—R₄₉ group wherein Z₁ represents a single bond, CH₂—CH₂, CH═CH, C≡C, O, S or NR₅₀, particularly a single bond, CH₂—CH₂, C≡C, O or NR₅₀; m represents an integer between 1 and 6, particularly between 1 and 4; R₅₀ represents a hydrogen atom or a (C₁-C₆)alkyl group; and R₄₉ represents a halogen atom, OR₂₀, NR₂₂R₂₃, C(O)R₂₄, CO₂R₂₅, OC(O)R₂₆, NR₂₉C(O)R₃₀, C(O)NR₃₁R₃₂, NR₃₃CO₂R₃₄, OC(O)NR₃₅R₃₆, NR₃₇CONR₃₈R₃₉ or OCO₂R₄₀; particularly OR₂₀, NR₂₂R₂₃, CO₂R₂₅, C(O)NR₃₁R₃₂, NR₃₃CO₂R₃₄, OC(O)NR₃₅R₃₆, NR₃₇CONR₃₈R₃₉ or OCO₂R₄₀; notably NR₂₂R₂₃, NR₃₃CO₂R₃₄, or NR₃₇CONR₃₈R₃₉, or
a heterocycle having 5, 6 or 7 members, particularly 6 or 7 members, comprising 1 or 2 heteroatoms selected from N and O and comprising advantageously at least one nitrogen atom, preferably saturated or aromatic, particularly saturated, (the heterocycle can be in particular an optionally bridged piperazine (e.g. piperazine, 2,5-diazabicyclo[4.2.0]octane, 3,8-diazabicyclo[3.2.1]octane or 2,5-diazabicyclo[2.2.1]heptane), piperidine, perhydropyrrolo[3,4-c]pyrrole, tetrahydropyridine or pyrrolidine ring; particularly a piperazine, piperidine, morpholine, diazepane (e.g. 1,4-diazepane) or pyridine ring) optionally substituted by one or more substituents selected from:
   a halogen atom,
   a (C₁-C₆)alkyl group optionally substituted by one or more groups selected from a halogen atom, OR₁₆, NR₁₈R₁₉, a C₃ to C₆ monocyclic carbocycle (particularly saturated) and a 3- to 6-membered monocyclic heterocycle (particularly saturated) comprising 1 or 2 heteroatoms selected from N and O.
   oxo (═O), OR₂₀, NR₂₂R₂₃, C(O)R₂₄, CO₂R₂₅, OC(O)R₂₆, NR₂₉C(O)R₃₀, C(O)NR₃₁R₃₂, NR₃₃CO₂R₃₄, OC(O)NR₃₅R₃₆, NR₃₇CONR₃₈R₃₉ or OCO₂R₄₀ groups; and more particularly oxo (═O), OR₂₀, NR₂₂R₂₃, CO₂R₂₅, C(O)NR₃₁R₃₂, NR₃₃CO₂R₃₄, OC(O)NR₃₅R₃₆, NR₃₇CONR₃₈R₃₉ or OCO₂R₄₀ groups,
   a C₃ to C₆ carbocycle optionally substituted by one or more groups selected from a halogen atom, a (C₁-C₆)alkyl group, oxo (═O), OR₄₁ and NR₄₃R₄₄,
   a 3- to 6-membered heterocycle comprising 1 or 2 heteroatoms selected from N and O, particularly saturated or unsaturated, particularly non-aromatic, preferably saturated, optionally substituted by one or more groups selected from a halogen atom, a (C₁-C₆)alkyl group, oxo (═O), OR₄₅ and NR₄₇R₄₈, and
an —O(CH₂)ₙO— group wherein n represents an integer equal to 2 or 3 (the two oxygens of this group can be attached to the same atom or to two different atoms, advantageously they are attached to the same atom, in particular to the same carbon atom, making it possible in this case to form a cyclic acetal),
   wherein:
R₁₆, R₁₈ to R₂₀, R₂₂ to R₂₆, R₂₉ to R₄₀, R₄₅ and R₄₇ to R₄₈ represent, independently of each other, a hydrogen atom, a (C₁-C₆)alkyl, aryl, or aryl-(C₁-C₆)alkyl group, particularly a hydrogen atom or a (C₁-C₆)alkyl group,
   the aryl ring of these groups being a phenyl group and being optionally substituted by one or more groups selected from a halogen atom and a (C₁-C₈)alkyl group, or
R₂₂ and R₂₃, R₃₁ and R₃₂, R₃₅ and R₃₆, R₃₈ and R₃₉, R₄₃ and R₄₄, and/or R₄₇ and R₄₈ together form, with the nitrogen atom that bears them, a 5- or 6-membered nitrogen containing heterocycle, particularly non-aromatic, preferably saturated, optionally comprising 1 heteroatom in addition to the nitrogen atom selected from N and O, such as a piperazine, piperidine, morpholine or pyrrolidine ring, the heterocycle being optionally substituted by one or more groups selected from a halogen atom, a (C₁-C₆)alkyl group, and oxo (═O).

According to a particular embodiment of the invention, at least one of R₉ and R₁₀ does not represent a hydrogen atom. According to another particular embodiment of the invention, at least one of R₉ and R₁₀ does not represent a hydrogen atom or a halogen atom.

According to still another embodiment of the invention, one of R₉ and R₁₀ represents a hydrogen atom and the other does not represent a hydrogen atom, and particularly does not represent a hydrogen atom or a halogen atom.

According to a particular embodiment, Z═O, Y represents a CRy group wherein Ry is as defined according to one of the previous definitions and in particular wherein Y═CH or CF and R₁═R₂═R₃═R₄═R₁′═R₂′═R₃′═R₄′═H.

The compounds of formula (I) thus correspond to the compounds of the following formula (Ia):

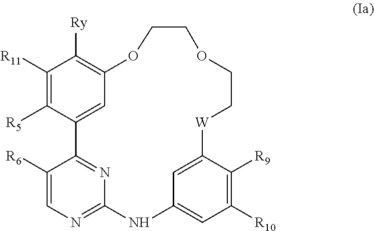

and the pharmaceutically acceptable salts and/or solvates thereof,
wherein:
   Ry is as defined above and advantageously represents a hydrogen atom, a halogen atom, a (C₁-C₆)alkyl or (C₁-C₆)haloalkyl group; particularly a hydrogen atom or a halogen atom (e.g. F),
   a R₅ and R₆ are as defined according to one of the previous definitions and preferably represent, independently of each other, a hydrogen atom or a (C₁-C₆)alkyl group,
   R₁₁ is as defined above and advantageously represents a hydrogen or halogen atom (e.g. F), and
   R₉ and R₁₀ are as defined according to any one of the preceding embodiments.

The compounds of the present invention can be selected particularly from compounds 1 to 46 described in the examples below, in the form of the free base thereof or of a pharmaceutically acceptable salt and/or solvate thereof, particularly in the form of the hydrochloride thereof.

The present invention also relates to a compound of formula (I) as defined above, for use as a drug, in particular intended for the treatment of cancer.

The present invention also relates to the use of a compound of formula (I) as defined above, for the manufacture of a drug, in particular intended for the treatment of cancer.

The present invention also relates to a method for treating cancer, comprising the administration to a person in need thereof of an effective dose of a compound of formula (I) as defined above.

The cancer can be more particularly in this case colon cancer, breast cancer, kidney cancer, liver cancer, pancreatic cancer, prostate cancer, glioblastoma, lung cancer, neuroblastoma, inflammatory myofibroblastic tumor, lymphoma, leukemia, myelodysplastic syndrome, myelofibrosis, ovarian cancer, cancer of the head and neck.

The present invention also relates to a pharmaceutical composition comprising at least one compound of formula (I) as defined above, and at least one pharmaceutically acceptable excipient.

The pharmaceutical compositions according to the invention can be formulated in particular for oral administration or for administration by injection, said compositions being intended for mammals, including humans.

The active ingredient can be administered in unit dosage forms, mixed with standard pharmaceutical excipients, to animals or to human beings.

The suitable oral unit dosage forms include tablets, capsules, powders, granules and oral solutions or suspensions.

When a solid composition is prepared in tablet form, the principal active ingredient is mixed with a pharmaceutical vehicle such as gelatin, starch, lactose, magnesium stearate, talc, gum arabic or analogues. The tablets can be coated with sucrose or other suitable materials or they can be treated so that they have a prolonged or delayed activity and that they continuously release a predetermined quantity of active ingredient.

A capsule preparation is obtained by mixing the active ingredient with a diluent and pouring the mixture obtained into soft or hard capsules.

A preparation in syrup or elixir form can contain the active ingredient together with a sweetener, an antiseptic, as well as a flavor enhancer and a suitable dye.

The water-dispersible powders or granules can contain the active ingredient mixed with dispersants or wetting agents, or suspending agents, as well as with flavor enhancers or sweeteners.

For administration by injection, one uses aqueous suspensions, isotonic saline solutions or sterile solutions for injection that contain pharmacologically compatible dispersants and/or wetting agents.

The active ingredient can be also formulated in microcapsule form, optionally with one or more additive excipients.

The compounds of the invention as active ingredients can be used at doses between 0.01 mg and 1000 mg per day, given in a single dose once per day or administered in several doses throughout the day, for example twice a day in equal doses. The dose administered per day is advantageously between 5 mg and 500 mg, even more advantageously between 10 mg and 200 mg. It may be necessary to use doses outside these ranges, which would be obvious to the person skilled in the art.

The pharmaceutical compositions according to the invention can further comprise at least one other active ingredient, such as an anti-cancer agent.

The present invention also relates to a pharmaceutical composition comprising:
(i) at least one compound of formula (I) as defined above, and
(ii) at least one other active ingredient, such as an anti-cancer agent,
   as a combination product for simultaneous, separate or sequential use.

The present invention also relates to a pharmaceutical composition as defined above for use as a drug, particularly intended for the treatment of cancer.

The present invention also relates to a method for treating cancer, comprising the administration to a person in need thereof of an effective dose of a pharmaceutical composition as defined above.

The present invention also relates to the methods for preparing the compounds of formula (I) according to the invention.

The present invention thus relates to a first method for preparing a compound of formula (I) comprising the coupling reaction between:
a compound of the following formula (II):

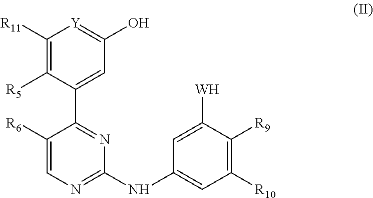

wherein W, Y, R₅, R₆, R₉, R₁₀ and R₁₁ are as defined above, and
a compound of the following formula (III):

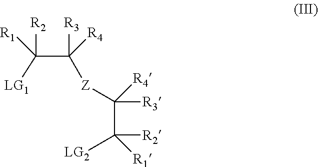

wherein Z, R₁, R₂, R₃, R₄, R₁′, R₂′, R₃′ and R₄′ are as defined above and LG₁ and LG₂ each represent, independently of each other, a leaving group.

By “leaving group” is meant, in the meaning of the present invention, a chemical group that can be easily displaced by a nucleophile during a nucleophilic substitution reaction, the nucleophile being in the present case an alcohol or a thiol. Such a leaving group can be more particularly a halogen atom such as a chlorine or bromine atom or a sulfonate. The sulfonate can be in particular an —OSO₂—R₅₁ group wherein R₅₁ represents a (C₁-C₆)alkyl, aryl, aryl-(C₁-C₆)alkyl or (C₁-C₆)alkykaryl group, said group being optionally substituted by one or more halogen atoms such as fluorine atoms. The sulfonate can be in particular a mesylate (—OS(O₂)—CH₃), a triflate (—OS(O)₂—CF₃) or a tosylate (—OS(O)₂-(p-Me-C₆H₄)).

Groups LG₁ and LG₂ will represent more particularly a halogen atom such as a bromine.

The coupling (or macrocyclization) reaction will be carried out advantageously in the presence of a base such as potassium carbonate or sodium carbonate. Dimethylformamide can be used as the reaction solvent.

Such a method is more particularly used when W═O.

Such a method is illustrated in greater detail in the following Schemes 1, 2 and 3 and in the experimental section below.

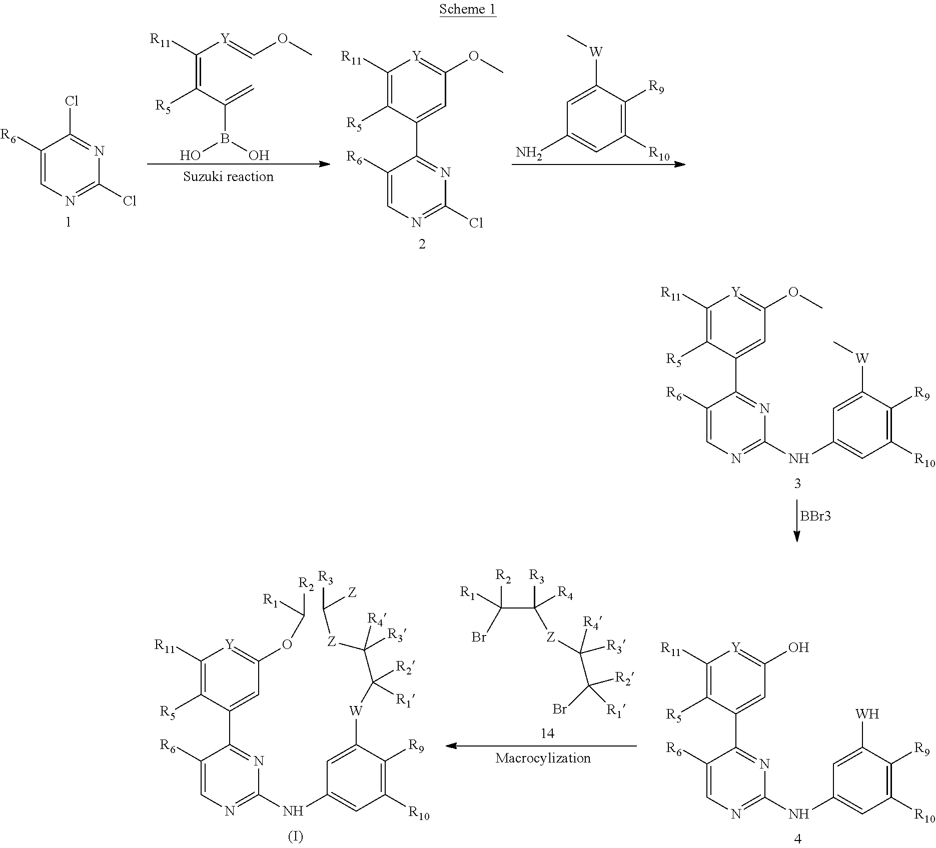

The first step is a catalytic coupling reaction such as Suzuki reaction between a boronate, in acid (as illustrated) or ester form, and an optionally substituted derivative of 2A-dichloropyrimidine. In this case, these compounds are heated together with a palladium catalyst such as Pd(dppf)Cl₂ or Pd(PPh₃)₄, an organic base such as triethylamine or an alcoholate (in particular (C₁-C₆)alkyl-OM with M=Na, K or Li), or an inorganic base such as sodium carbonate, potassium carbonate or cesium carbonate. Toluene, benzene, tetrahydrofuran, dioxane or mixtures thereof can be used as solvents. The preferred reaction temperatures are between 20° C. and 100° C.

The transformation of compounds 2 into compounds 3 can be carried out by an aromatic nucleophilic substitution reaction. In this case, the nucleophiles used are functionalized anilines which are reacted in the presence of an acid such as hydrochloric acid, preferably in a polar solvent such as n-butanol. If need be, these reactions can be carried out in a microwave reactor, particularly in a polar solvent such as N-methyl-2-pyrrolidinone. The preferred reaction temperatures are between 20° C. and 150° C.

Compounds 3 can be transformed into compounds 4 by a demethylation reaction in the presence of BBr₃, particularly in an anhydrous solvent such as dichloromethane, preferably at a temperature between −78° C. and 100° C. Compounds 4 are transformed into compounds of general formula (I) by a macrocyclization reaction in the presence of dibrominated compounds 14.

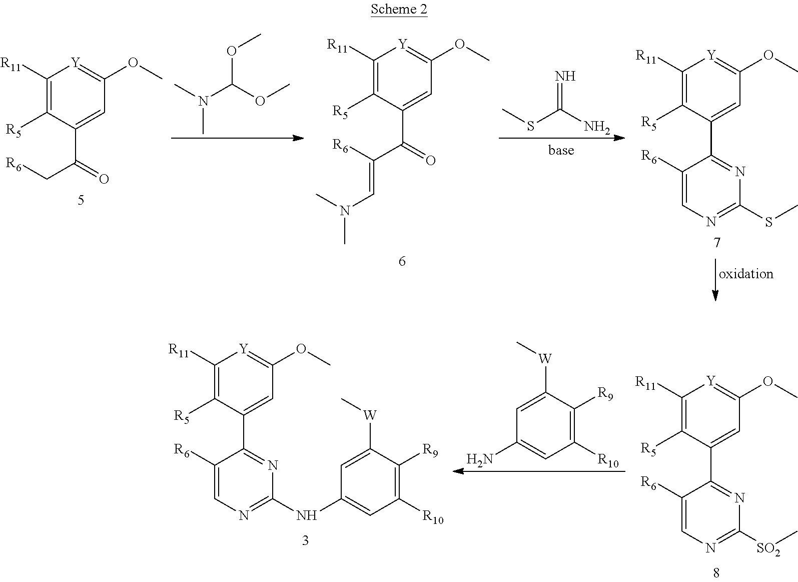

Scheme 2 presents a second synthetic pathway of compounds 3.

The reaction of the acetophenone 5 variously substituted with N,N-dimethylformamide dimethyl acetal gives access to compounds 6. These reactions are typically carried out without solvent or in an anhydrous polar solvent such as dimethylformamide, particularly at a temperature between 0° C. and 170° C.

The second step of cyclization between compound 6 and S-methylisothiourea hemisulfate is typically carried out in the presence of an organic base such as triethylamine, potassium acetate or sodium acetate or an alcoholate, or an inorganic base such as sodium carbonate, potassium carbonate or cesium carbonate. This reaction can be carried out at a temperature between 20° C. and 200° C., particularly in a polar solvent, such as N,N-dimethylformamide, or without solvent in a microwave reactor.

The compounds 7 obtained are subjected to an oxidation of their thiomethoxy function, typically by the use of m-CPBA, oxone or any other equivalent oxidizing agent, to lead to the formation of the corresponding sulfone 8.

These compounds 8 are then engaged in a nucleophilic substitution reaction in the presence of an aniline to form compounds 3.

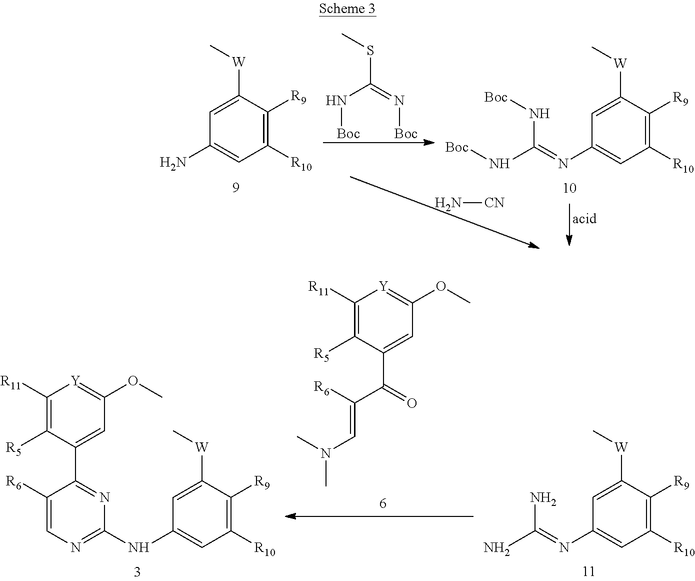

Scheme 3 presents a third synthetic pathway of compounds 3.

The anilines 9 are transformed into protected guanidine derivatives 10 by one of the usual methods well known to the person skilled in the art, derivatives 10 which are deprotected in acidic conditions to give deprotected guanidines 11, for example by using trifluoroacetic acid, particularly for periods varying typically from 1 to 3 days, particularly at temperatures varying between 0° C. and 40° C.

The anilines 9 can also be transformed directly into deprotected guanidines 11 by reaction with cyanamide, in the presence of an acid such as HCl, H₂SO₄ or HCOOH, particularly in the absence of solvent or in the presence of a solvent such as toluene or ethanol, particularly at a temperature between 20° C. and 100° C. This reaction can be carried out by the methods and techniques well known the person skilled in the art.

The guanidines 11 thus obtained are subjected to a condensation reaction with compounds 6 to form the corresponding compounds 3.

The present invention also relates to a second method for preparing a compound of formula (I) comprising the cyclization reaction of a compound of the following formula (VIa) or (VIb):

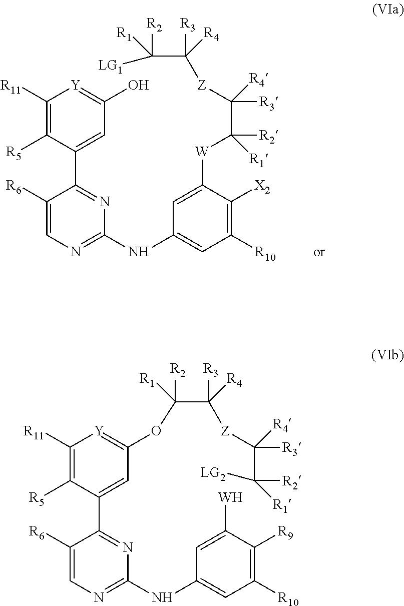

wherein W, Y, Z, R₁ to R₆, R₉ to R₁₁ and R₁′ to R₄′ are as defined above, and LG₁ and LG₂ each represent, independently of each other, a leaving group, such as a halogen atom and in particular a bromine atom.

The cyclization reaction will be carried out advantageously in the presence of a base such as potassium hydroxide or sodium hydroxide. Tetrahydrofuran can be used as the reaction solvent.

Such a method is more particularly used when W═S.

Such a method is illustrated in greater detail in the following Scheme 4 and in the experimental section below.

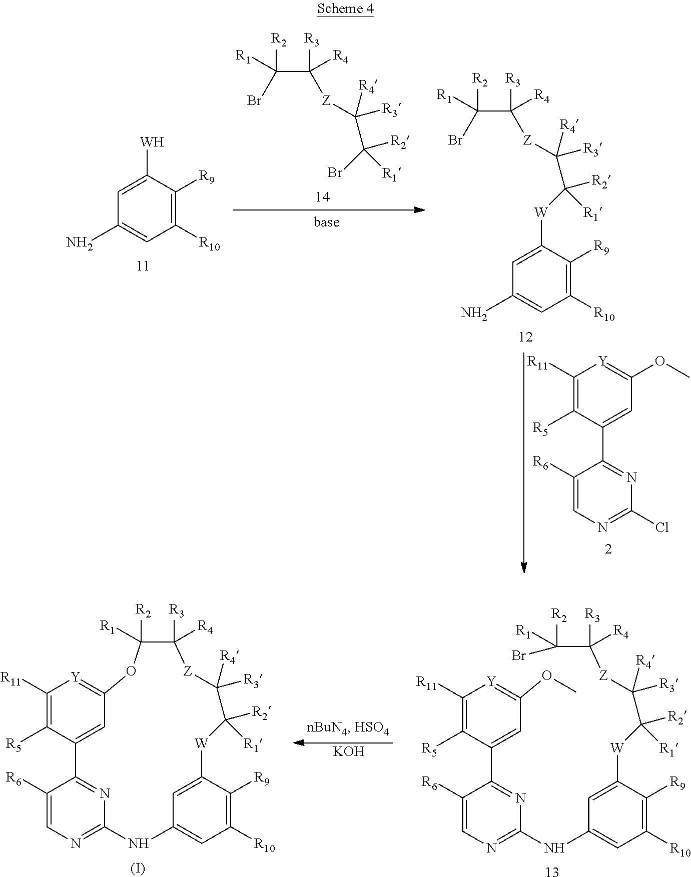

Compound 11 is transformed into compound 12 by reaction with the dibrominated derivative 14. This reaction can be carried out in the presence of an organic or inorganic base such as for example Et₃N, iPr₂NEt, NaH, pyridine, Cs₂CO₃, K₂CO₃ or Na₂CO₃, optionally in the presence of a salt as catalyst which can be KI, Bu₄NI, CuI, LiI, AgBF₄, AgClO₄, Ag₂CO₃, KF, Bu₄NF or CsF or optionally in the presence of a phase transfer agent such as nBu₄N, HSO₄. The reaction can be carried out in an anhydrous polar solvent such as tetrahydrofuran, dimethylformamide, dimethylsulfoxide, acetone or a mixture thereof, particularly at a temperature between −20° C. and 140° C. The reaction can also be carried out in a “screw-capped or sealed test tube” heated by thermal energy or microwave energy, at temperatures between 80° C. and 180° C.

Compounds 12 can be transformed into compounds 13 by an aromatic nucleophilic substitution reaction. The reaction can be carried out in the presence of an acid such as hydrochloric acid, particularly in a polar solvent such as n-butanol. If need be, these reactions can be carried out in a microwave reactor, for example in a polar solvent such as N-methyl-2-pyrrolidinone. Typically, the preferred operating conditions involve temperatures between 20° C. and 150° C.

Compound 13 is then cyclized to give a compound of general formula (I) according to reaction conditions similar to the transformation of compound 11 into compound 12.

The present invention also relates to a third method for preparing a compound of formula (I) wherein R₉ and/or R₁₀ represents an optionally substituted (C₁-C₆)alkoxy, optionally substituted (C₁-C₆)thioalkoxy or NR₁₄R₁₅ group or an optionally substituted heterocycle comprising a heteroatom directly attached to the phenyl ring, comprising the coupling between a compound of the following formula (IVa) or (IVb):

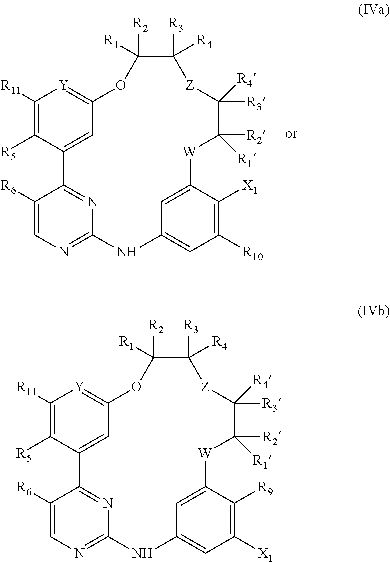

wherein W, Y, Z, R₁ to R₆, R₉ to R₁₁ and R₁′ to R₄′ are as defined above and X₁ represents a halogen atom such as Br, Cl or I, particularly Br, and respectively a compound of formula R₉H or R₁₀H wherein R₉ and R₁₀ are as defined above.

This reaction can be carried out in the presence of an organic or inorganic base, such as Et₃N, iPr₂NEt, NaH, pyridine, Cs₂CO₃, Na₂CO₃ or K₂CO₃, optionally in the presence of a salt as catalyst such as KI, CuI, Bu₄NI, LiI, AgBF₄, AgClO₄, Ag₂CO₃, KF, Bu₄NF or CsF. The solvent used will be preferably an anhydrous polar solvent such as tetrahydrofuran, dimethylformamide, dimethylsulfoxide, acetone or a mixture thereof. The reaction can advantageously be carried out at a temperature between −20° C. and 140° C. The choice of experimental conditions and of reagents for carrying out this reaction is obvious depending on the nature of the nucleophiles R₉H and R₁₀H and will be carried out according to the methods and techniques well known to the person skilled in the art.

The reaction can also be carried out in a “screw-capped or sealed test tube” heated by thermal energy or microwave energy, particularly at temperatures between 80° C. and 180° C. according to the reference (J. Org. Chem. 2009, 74, 5075-5078).

This reaction can also be carried out by catalytic coupling such as described in the reference (Org. Lett. 2002, 17, 2885-2888). This reaction is carried out in the presence of a catalytic quantity of a palladium complex such as (dppf)₂PdCl₂.CH₂Cl₂. The coupling reaction is carried out advantageously at temperatures between 25° C. and 100° C. The solvent used will be preferably a polar aprotic solvent such as tetrahydrofuran or dioxane.

25

The present invention also relates to a fourth method for preparing a compound of formula (I), wherein R₉ and/or R₁₀ represents an optionally substituted (C₁-C₆)alkyl, optionally substituted (C₂-C₆)alkenyl or optionally substituted (C₂-C₆)alkynyl group, an optionally substituted carbocycle or an optionally substituted heterocycle attached to the phenyl ring by means of a carbon atom, comprising the coupling between a compound of the following formula (Va) or (Vb):

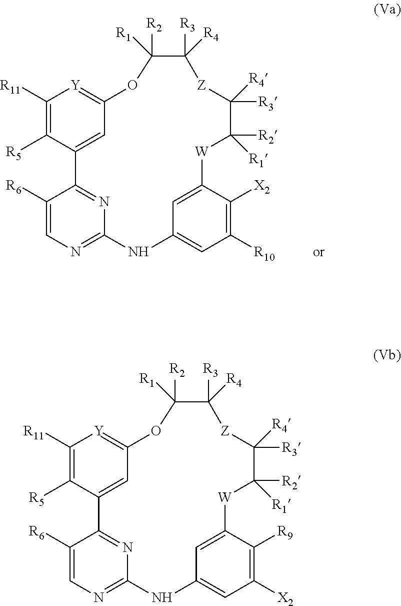

wherein W, Y, Z, R₁ to R₆, R₉ to R₁₁ and R₁′ to R₄′ are as defined above and X₂ represents Br, Cl, I or OTf (OSO₂CF₃),
and respectively a compound of formula R₉—BR₅₂R₅₃ or R₁₀-BR₅₂R₅₃ wherein R₉ and R₁₀ are as defined above and R₅₂ and R₅₃ represent, independently of each other, an OH, (C₁-C₆)alkyl or (C₁-C₆)alkoxy group or R₅₂ and R₅₃ together form an —X₃— or —O—X₃—O— chain wherein X₃ represents a divalent hydrocarbon group comprising 2 to 15, particularly 2 to 10, carbon atoms,

The reaction conditions for such a coupling are well known to the person skilled in the art as it is a Suzuki coupling.

This reaction is advantageously carried out in the presence of a palladium-based catalyst, for example palladium acetate, tetrakis(triphenylphosphine)palladium(0) or tris(dibenzylideneacetone)dipalladium(0). A phosphine such as triphenylphosphine or tricyclohexylphosphine can also be present.

An organic or inorganic base can be present, such as an alcoholate (in particular (C₁-C₆)alkyl-OM with M=Na, K or Li), NMP (N-methyl-morpholine), Et₃N, iPr₂NEt, K₃PO₄, NaH, Cs₂CO₃, Na₂CO₃ or K₂CO₃.

A polar solvent can be used such as tetrahydrofuran, dimethylformamide, acetonitrile, acetone, methylethylketone, ethanol, dimethyl ether, dioxane, water or a mixture thereof. The reaction can be advantageously carried out at a temperature between 20° C. and 140° C.

The —BR₅₂R₅₃ group can be for example a —B(OH)₂, —B((C₁-C₆)alkyl)₂, —B(O(C₁-C₆)alkyl)₂ (e.g. —B(OiPr)₂),

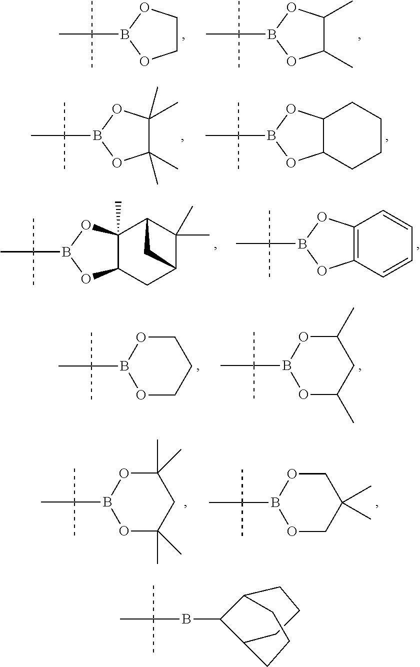

group, etc.

The present invention also relates to fifth method for preparing a compound of formula (I) wherein R₉ and/or R₁₀ represents —Z₁—(CH₂)ₘ—R₄₉ with Z1 representing CH₂—CH₂, CH═CH or C≡C, comprising the following steps:
(1) Sonogashira coupling between a compound of formula (Va) or (Vb) as defined above
   and a compound of formula HC≡C—(CH₂)ₘ—R₄₉ wherein m and R₄₉ are as defined above,
   to give a compound of formula (I) wherein R₉ or R₁₀ represents —C≡C—(CH₂)ₘ—R₄₉, and
(2) optionally reduction of the alkyne function of the compound of formula (I) obtained in the preceding step to give a compound of formula (I) wherein R₉ or R₁₀ represents —CH═CH—(CH₂)ₘ—R₄₉ or —(CH₂)ₘ₊₂—R₄₉.

Step (1):

Sonogashira coupling is a reaction well known to the person skilled in the art who will be able to determine the reaction conditions thereof. It is described particularly in the article by Sonogashira et al. in Tetrahedron Lett. 1975, 16, 4467-4470.

This coupling involves a reaction between an acetylene derivative and a halide or an aryl triflate catalyzed by complexes of palladium and copper.

Such a reaction is typically carried out under inert atmosphere, in the presence of a catalytic quantity of a palladium complex (for example PdCl₂(PPh₃)₂ or Pd(PPh₃)₄), a catalytic quantity of a copper salt (for example CuI), and a base which can be organic, such as triethylamine or DIPEA (diisopropylethylamine), or inorganic, such as sodium carbonate, potassium carbonate or cesium carbonate. The operational conditions generally include reaction temperatures between 20° C. and 45° C., particularly in solvents including dimethylformamide, tetrahydrofuran, dioxane, diethyl ether or a mixture thereof.

Step (2):

The reduction reaction of the triple bond of the alkyne function C≡C to give a double bond CH═CH or a single bond CH₂—CH₂ is well known to the person skilled in the art who will be able to determine the reaction conditions thereof.

This reduction can be carried out for example by hydrogen in the presence of a catalyst, for example of the palladium on carbon type, particularly in a common ethanol-type solvent, to obtain a single bond CH₂—CH₂.

Finally, the present invention relates to a sixth method for preparing a compound of formula (I) wherein R₉ and/or R₁₀ represents a CO₂R₅₄ or CONR₅₅R₅₆ group, which comprises at least one of the following steps:
(a) to obtain a compound of formula (I) wherein R₉ and/or R₁₀ represents a CO₂H group, the reaction of a compound of formula (I) wherein R₉ and/or R₁₀ represents a halogen atom with CO₂;
(b) to obtain a compound of formula (I) wherein R₉ and/or R₁₀ represents a CO₂R₅₄ group wherein R₅₄≠H, the substitution reaction of a compound of formula (I) wherein R₉ and/or R₁₀ represents a CO₂H group, optionally obtained according to step (a), optionally in an activated form, with an alcohol of formula R₅₄OH;
(c) to obtain a compound of formula (I) wherein R₉ and/or R₁₀ represents a CONR₅₅R₅₆ group, the substitution reaction of a compound of formula (I) wherein R₉ and/or R₁₀ represents a CO₂H group, optionally obtained according to step (a), optionally in an activated form, with an amine of formula HNR₅₅R₅₆.

Step (a):

This reaction will be advantageously carried out in the presence of a base such as ((C₁-C₆)alkyl)Li (e.g. BuLi). Tetrahydrofuran can be used as the reaction solvent. The reaction will be carried out preferably at a temperature below 0° C., particularly below −50° C., e.g. at about −78° C.

Steps (b) and (c):

By “activated form” of the CO₂H group (carboxylic acid) is meant, in the meaning of the present invention, a carboxylic acid group wherein the OH moiety of the COOH function has been replaced by an activated leaving group (LG) enabling the coupling of the CO₂H group in an activated form with a hydroxyl (OH) or amino (NH) function by formation of an ester (C(O)—O) or amide (C(O)—N) bond and release of the LG-H compound. The activated forms can be activated esters, activated amides, acyl anhydrides or halides such as acyl chlorides. Activated esters include derivatives formed by the reaction of the carboxylic acid group with N-hydroxybenzotriazole or N-hydroxysuccinimide.

The reactions of steps (b) and (c) are well known to the person skilled in the art.

The substitution reaction with the alcohol will be carried out preferably with a compound of formula (I) bearing a carboxylic acid function (CO₂H) in activated form, particularly in the form of an acyl chloride.

The substitution reaction with the amine can be carried out with a compound of formula (I) bearing a carboxylic acid function (CO₂H) in activated form, particularly in the form of an acyl chloride, or bearing a non-activated carboxylic acid function (CO₂H) in peptide coupling conditions well known to the person skilled in the art.

The peptide coupling can thus be carried in the presence of a coupling agent, such as diisopropylcarbodiimide (DIC), dicyclohexylcarbodiimide (DCC), 1-(3-dimethylaminopropyl)-3-ethylcarbodiimide hydrochloride (EDC), carbonyldiimidazole (CDI), 2-(1H-benzotriazole-1-yl)-1,1,3,3-tetramethyluronium hexafluorophosphate (HBTU), 2-(1H-benzotriazole-1-yl)-1,1,3,3-tetramethyluronium tetrafluoroborate (TBTU), O-(7-azobenzotriazol-1-yl)-1,1,3,3-tetramethyluronium hexafluorophosphate (HATU), (benzotriazol-1-yloxy)tripyrrolodinophosphonium hexafluorophosphate (PyBOP) or propylphosphonic anhydride, optionally combined with a coupling aid such as N-hydroxysuccinimide (NHS), N-hydroxybenzotriazole (HOBt), 3,4-dihydro-3-hydroxy-4-oxo-1,2,3-benzotriazole (HOOBt), 1-hydroxy-7-azabenzotriazole (HAt), N-hydroxysulfosuccinimide (sulfo NHS), dimethylaminopyridine (DMAP), diisopropylethylamine (DIEA) or N-methylmorpholine (NMM). Particularly, it can be carried out in the presence of propylphosphonic anhydride and a base such as DIEA. This reaction can also be carried out under microwaves.

The six general methods described above can be supplemented, if need be, by any standard operations described in the literature, known to the person skilled in the art or exemplified in the experimental section, particularly by additional functionalization and/or protection/deprotection reactions.

One or more additional steps of salification and/or of solvation can be carried out at the end of these three methods to obtain a pharmaceutically acceptable salt and/or solvate of the compound of formula (I).

The salification step can be carried out in conditions well known to the person skilled in the art, in the presence of a pharmaceutically acceptable acid or base.

When the compound of formula (I) is in solvate form, this solvation has generally taken place in the final step of the method, the solvent of the solvate form being in this case the reaction medium solvent.

The compound of formula (I) obtained by one of these six methods mentioned above can be separated from the reaction medium by methods well known to the person skilled in the art, such as for example by extraction, solvent evaporation or by precipitation and filtration.

The compound of formula (I) can be further purified if necessary by techniques well known to the person skilled in the art, such as by recrystallization if the compound is crystalline, by distillation, by column chromatography on silica gel or by high-performance liquid chromatography (HPLC).

The invention is illustrated by the following non-limiting examples.

### EXAMPLES

1. Synthesis of the Compounds According to the Invention

The following abbreviations were used:

DMSO: Dimethylsulfoxide

EI: Electron impact

LCMS: Liquid chromatography coupled to mass spectrometry

NMR: Nuclear magnetic resonance

Compound 1:

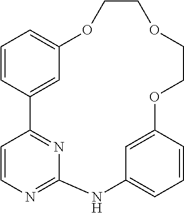 [SMILES: c1cc2cc(c1)OCCOCCOc1cccc(c1)-c1ccnc(n1)N2]

Step 1: 3-(2-chloropyrimidin-4-yl)phenol (intermediate 1)

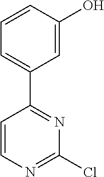 [SMILES: Oc1cccc(-c2ccnc(Cl)n2)c1]

To 5 g (33.6 mmol) of 2,4-dichloropyrimidine in 250 mL of anhydrous tetrahydrofuran (THF) is added 4.41 g (32 mmol) of (3-hydroxyphenyl)boronic acid. The reaction mixture is stirred at room temperature for 10 minutes then 8.47 g (80 mmol) of sodium carbonate dissolved in 20 mL of water then 462 mg (1.59 mmol) of tetrakis(triphenylphosphine)palladium(0) is added at room temperature. The reaction mixture is stirred at 90° C. for 16 hours. Ethyl acetate is added and the organic phase is washed with saturated sodium chloride solution, dried over magnesium sulfate and filtered. The filtrate is evaporated and the residue purified by silica column chromatography using Companion® (eluent: cyclohexane/ethyl acetate: 0 to 10%) to afford 1.54 g (23%) of 3-(2-chloropyrimidin-4-yl)phenol as a white solid.

LCMS (EI, m/z): (M+1) 207.62

¹H NMR: dH ppm (400 MHz, DMSO): 9.82 (1H, s, OH), 8.79-8.80 (1H, d, CHₐᵣₒₘ), 8.06-8.08 (1H, d, CHₐᵣₒₘ), 7.61-7.62 (2H, m, CHₐᵣₒₘ), 7.35-7.39 (1H, d, CHₐᵣₒₘ), 6.99-7.01 (1H, t, CHₐᵣₒₘ)

Step 2: 3-(2-((3-hydroxyphenyl)amino)pyrimidin-4-yl)phenol (intermediate 2)

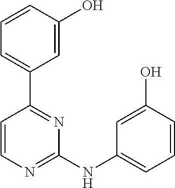 [SMILES: Oc1cccc(Nc2nccc(-c3cccc(O)c3)n2)c1]

In a microwave reactor are mixed 150 mg (0.726 mmol) of 3-(2-chloropyrimidin-4-yl)phenol, 103 mg (0.944 mmol) of 3-aminophenol and 0.5 mL of N-methyl-2-pyrrolidinone. The reaction mixture is heated to 150° C. for 15 minutes. After returning to room temperature, the reaction mixture is hydrolyzed by addition of water and extracted with ethyl acetate. The organic phase is dried over sodium sulfate, filtered and concentrated. The residue obtained is purified by chromatography on silica gel (eluent: dichloromethane/methanol: 95:5) to afford 129 mg (63%) of 3-(2-((3-hydroxyphenyl)amino)pyrimidin-4-yl)phenol as a yellow solid.

LCMS (EI, m/z): (M+1) 280.29

¹H NMR: dH ppm (400 MHz, DMSO): 9.68 (1H, s, OH), 9.50 (1H, s, NH), 9.24 (1H, s, OH), 8.50-8.51 (1H, d, CHₐᵣₒₘ), 7.54-7.58 (2H, m, CHₐᵣₒₘ), 7.28-7.35 (4H, m, CHₐᵣₒₘ), 7.07-7.09 (1H, m, CHₐᵣₒₘ), 6.93-6.95 (1H, m, CHₐᵣₒₘ), 6.37-6.39 (1H, d, CHₐᵣₒₘ)

Step 3: Compound 1

To a stirred solution of 47.7 mg (0.171 mmol) of 3-(2-((3-hydroxyphenyl)amino)pyrimidin-4-yl)phenol in 12.5 mL of N,N-dimethylformamide is added 54.3 mg (0.393 mmol) of potassium carbonate then 21.47 μL (0.171 mmol) of 1-bromo-2-(2-bromoethoxy)ethane in 0.2 mL of N,N-dimethylformamide for one hour. The reaction mixture is stirred at 80° C. for 5 hours. After returning to room temperature, the solvent is evaporated, water is added and the solid formed is filtered and dried under vacuum to afford 5.3 mg (9%) of compound 1 as a beige powder.

LCMS (EI, m/z): (M+1) 350.38

¹H NMR: dH ppm (400 MHz, DMSO): 9.83 (1H, s, NH), 8.68 (1H, m, CHₐᵣₒₘ), 8.58-8.59 (1H, d, CHₐᵣₒₘ), 8.29 (1H, S, CHₐᵣₒₘ), 7.73-7.75 (1H, m, CHₐᵣₒₘ), 7.42-7.50 (2H, m, CHₐᵣₒₘ), 7.10-7.21 (2H, m, CHₐᵣₒₘ), 6.84-6.85 (1H, m, CHₐᵣₒₘ), 6.53-6.55 (1H, m, CHₐᵣₒₘ), 4.31-4.35 (2H, t, CH₂), 4.04 (2H, m, CH₂), 3.93 (4H, m, CH₂).

Compound 2:

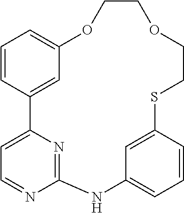 [SMILES: c1cc2cc(c1)SCCOCCOc1cccc(c1)-c1ccnc(n1)N2]

Step 1: 3-((2-(2-bromoethoxy)ethyl)thio)aniline (intermediate 3)

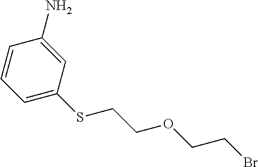 [SMILES: Nc1cccc(SCCOCCBr)c1]

To a stirred solution of 3 g (23.9 mmol) of 3-aminobenzenethiol in 30 mL of NA dimethylformamide is added 3.97 g (28 mmol) of potassium carbonate then 5.56 g (23.9 mmol) of 1-bromo-2-(2-bromoethoxy)ethane. The reaction mixture is stirred at 25° C. for 4 hours. The reaction mixture is hydrolyzed by addition of water and extracted with ethyl acetate. The organic phase is dried over sodium sulfate, filtered and concentrated. The residue obtained is purified by chromatography on silica gel (eluent: ethyl acetate/cyclohexane: 70:30) to afford 1.4 g (21%) of 3-((2-(2-bromoethoxy)ethyl)thio)aniline in the form of an oil.

LCMS (EI, m/z): (M+1) 277.19

¹H NMR: dH ppm (400 MHz, DMSO): 7.01-7.05 (1H, s, CHₐᵣₒₘ), 6.69-6.72 (1H, m, 6.61-6.64 (1H, d, CHₐᵣₒₘ), 6.51-6.53 (1H, d, CHₐᵣₒₘ), 3.68-3.75 (2H, m, CH₂), 3.54-3.68 (4H, m, CH₂), 3.07-3.09 (2H, m, CH₂).

Step 2: 3-(2-((3-((2-(2-bromoethoxy)ethyl)thio)phenyl)amino)pyrimidin-4-yl)phenol (intermediate 4)

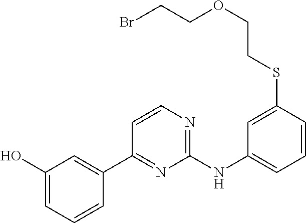 [SMILES: Oc1cccc(-c2ccnc(Nc3cccc(SCCOCCBr)c3)n2)c1]

In a microwave reactor are mixed 134 mg (0.484 mmol) of 3-((2-(2-bromoethoxy)ethyl)thio)aniline and 0.1 g (0.484 mmol) of 3-(2-chloropyrimidin-4-yl)phenol in 0.5 mL of N-methyl-2-pyrrolidinone. The reaction mixture is heated to 150° C. for 15 minutes. After returning to room temperature, the reaction mixture is hydrolyzed by addition of water and extracted with ethyl acetate. The organic phase is dried over sodium sulfate, filtered and concentrated. The residue obtained is purified by chromatography on silica gel (eluent: ethyl acetate/cyclohexane: 70:30) to afford 27 mg (12%) of 3-(2-((3-((2-(2-bromoethoxy)ethyl)thio)phenyl)amino)pyrimidin-4-yl)phenol as a yellow solid.

LCMS (EI, m/z): (M+1) 447.36

¹H NMR: dH ppm (400 MHz, DMSO): 9.70 (1H, s, NH), 9.70 (1H, s, CHₐᵣₒₘ), 8.54-8.55 (1H, d, CHₐᵣₒₘ), 7.91 (1H, s, CHₐᵣₒₘ), 7.67 (1H, d, CHₐᵣₒₘ), 7.54-7.58 (2H, m, CHₐᵣₒₘ), 7.33-7.34 (2H, m, CHₐᵣₒₘ), 7.26 (1H, t, CHₐᵣₒₘ), 6.94-6.96 (2H, m, CHₐᵣₒₘ), 3.66-3.68 (6H, m, CH₂), 3.14-3.17 (2H, m, CH₂).

Step 3: Compound 2

In a 50 mL round-bottom flask and under nitrogen are mixed 27 mg (0.060 mmol) of 3-(2-((3-((2-(2-bromoethoxy)ethyl)thio)phenyl)amino)pyrimidin-4-yl)phenol, 5.09 mg (0.091 mmol) of potassium hydroxide and 1.027 mg (3.02 μmol) of tetrabutylammonium hydrogensulfate in 1 mL of tetrahydrofuran. The reaction mixture is heated to 80° C. for 2 hours. After returning to room temperature, the reaction is hydrolyzed by addition of water and extracted with ethyl acetate. The organic phase is dried over sodium sulfate, filtered and concentrated. The residue obtained is purified by chromatography on silica gel (eluent: ethyl acetate/cyclohexane: 50:50) to afford 7 mg (31%) of compound 2 as a yellow solid.

LCMS (EI, m/z): (M+1) 366.44

¹H NMR: dH ppm (400 MHz, DMSO): 9.77 (1H, s, NH), 8.53-8.57 (2H, m, CHₐᵣₒₘ), 8.03 (1H, s, CHₐᵣₒₘ), 7.64-7.66 (1H, s, CHₐᵣₒₘ), 7.41-7.47 (2H, m, CHₐᵣₒₘ), 7.22-7.26 (1H, m, CHₐᵣₒₘ), 7.16-7.18 (1H, m, CHₐᵣₒₘ), 7.04-7.08 (2H, d, CHₐᵣₒₘ), 4.25-4.28 (2H, t, CH₂), 3.75-3.77 (2H, t, CH₂), 3.69-3.72 (2H, t, CH₂), 3.17-3.21 (2H, t, CH₂).

Compound 3:

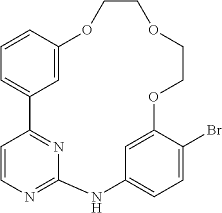 [SMILES: Brc1ccc2cc1OCCOCCOc1cccc(c1)-c1ccnc(n1)N2]

Step 1: (E)-3-(dimethylamino)-1-(3-methoxyphenyl)prop-2-en-1-one (intermediate 5)

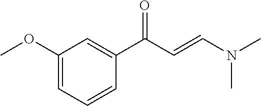

In a microwave reactor are mixed 2 g (13.32 mmol) of 1-(3-methoxyphenyl)ethanone and 11.50 mL (87 mmol) of N,N-dimethylformamide dimethyl acetal. The reaction mixture is heated to 200° C. for 10 minutes. After returning to room temperature, the residue is concentrated to afford 2.250 g (82%) of (E)-3-(dimethylamino)-1-(3-methoxyphenyl)prop-2-en-1-one as a yellow oil,

LCMS (EI, m/z): (M+1) 206.25

¹H NMR: dH ppm (400 MHz, DMSO): 7.69-7.72 (1H, d, CHₐᵣₒₘ), 7.44-7.46 (2H, m, CHₐᵣₒₘ and CH vinyl), 7.28-7.32 (1H, t, CHₐᵣₒₘ), 6.98-7.01 (1H, dd, CHₐᵣₒₘ), 5.67-5.70 (1H, d, CH vinyl), 3.84(3H, s, CH₃), 3.12 (3H, s, CH₃), 2.90 (3H, s, CH₃).

Step 2: 4-(3-methoxyphenyl)-2-(methylthio)pyrimidine (intermediate 6)

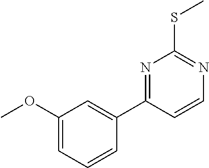

To 2.70 g (13.15 mmol) of (E)-3-(dimethylamino)-1-(3-methoxyphenyl)prop-2-en-1-one in 20 mL of N,N-dimethylformamide is added 5.49 g (19.73 mmol) of 5-methylisothiourea hemisulfate then 3.23 g (32.9 mmol) of potassium acetate. The reaction mixture is heated to 85° C. overnight. After returning to room temperature, the solvent is evaporated, the reaction is hydrolyzed by addition of water, basified and extracted with ethyl acetate. The organic phase is dried over sodium sulfate, filtered and concentrated to afford an oil which is used as such for the following step,

LCMS (EI, m/z): (M+1) 233.30

Step 3: 4-(3-methoxyphenyl)-2-(methylsulfonyl)pyrimidine (intermediate 7)

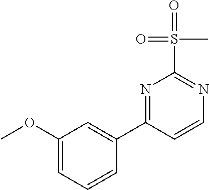

To 2.091 g (9 mmol) of 4-(3-methoxyphenyl)-2-(methylthio)pyrimidine in 30 mL of dichloromethane is added 4.66 g (27.0 mmol) of meta-chloroperbenzoic acid at 0° C. in small portions. The reaction mixture is stirred at 25° C. for 3 hours. The solvent is evaporated, the reaction is hydrolyzed by addition of water, basified and extracted with ethyl acetate. The organic phase is dried over sodium sulfate, filtered and concentrated to afford 0.600 g (25.2%) of 4-(3-methoxyphenyl)-2-(methylsulfonyl)pyrimidine as a yellow solid.

LCMS (EI, m/z): (M+1) 265.30

¹H NMR: dH ppm (400 MHz, DMSO): 9.11-9.12 (1H, d, CHₐᵣₒₘ), 8.42-8.43 (1H, m, CHₐᵣₒₘ), 7.88-7.90 (1H, d, CHₐᵣₒₘ), 7.81-7.82 (1H, t, CHₐᵣₒₘ), 7.51-7.55 (1H, t, CHₐᵣₒₘ), 7.21-7.24 (1H, dd, CHₐᵣₒₘ), 3.87 (3H, s, CH₃), 3.50 (3H, s, CH₃).

Step 4: N-(4-bromo-3-methoxyphenyl)-4-(3-methoxyphenyl)pyrimidin-2-amine (intermediate 8)

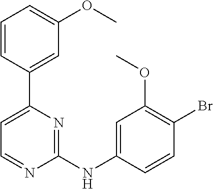 [SMILES: COc1cccc(-c2ccnc(Nc3ccc(Br)c(OC)c3)n2)c1]

In a microwave reactor are mixed 150 mg (0.57 mmol) of 4-(3-methoxyphenyl)-2-(methylsulfonyl)pyrimidine, 149 mg (0.73 mmol) of 4-bromo-3-methoxyaniline and 83 mg (0.73 mmol) of potassium tert-butylate in 3 mL of N,N-dimethylformamide. The reaction mixture is heated to 120° C. for 60 minutes. After returning to room temperature, the residue is concentrated to afford 165 mg (43%) of N-(4-bromo-3-methoxyphenyl)-4-(3-methoxyphenyl)pyrimidin-2-amine as a yellow solid.

LCMS (EI, m/z): (M+1) 387.24

¹H NMR: dH ppm (400 MHz, DMSO): 9.86 (1H, s, NH), 8.59-8.60 (1H, d, CHₐᵣₒₘ), 7.87 (1H, s, CHₐᵣₒₘ), 7.73-7.87 (2H, m, CHₐᵣₒₘ), 7.46-7.50 (3H, m, CHₐᵣₒₘ), 7.35-7.37 (1H, d, CHₐᵣₒₘ), 7.13-7.16 (1H, d, CHₐᵣₒₘ), 3.87 (3H, s, CH₃), 3.86 (3H, s, CH₃).

Step 5: 2-bromo-5-((4-(3-hydroxyphenyl)pyrimidin-2-yl)amino)phenol (intermediate 9)

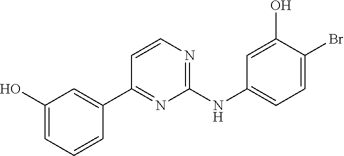 [SMILES: Oc1cccc(-c2ccnc(Nc3ccc(Br)c(O)c3)n2)c1]

To a solution of 5.67 g (14.68 mmol) of N-(4-bromo-3-methoxyphenyl)-4-(3-methoxyphenyl)pyrimidin-2-amine in 160 mL of dichloromethane is added 6.94 mL of tribromo-borane at −78° C. The reaction mixture is then stirred at 45° C. for 5 h then overnight at room temperature. 20 mL of methanol is added to the reaction mixture at 0° C. which is then heated to 35° C. for 25 minutes. The solid formed is filtered then washed twice with 20 mL of ether to afford 6.45 g (100%) of 2-bromo-5-((4-(3-hydroxyphenyl)pyrimidin-2-yl)amino)phenol as a yellow powder.

LC-MS (EI, m/z): (M+1) 440.10

¹H NMR: dH ppm (400 MHz, DMSO): 9.72 (1H, s, NH), 8.53-8.54 (1H, d, CHₐᵣₒₘ), 7.53-7.60 (3H, m, CHₐᵣₒₘ), 7.26-7.38 (4H, m, CHₐᵣₒₘ), 6.94-6.96 (1H, d, CHₐᵣₒₘ).

Step 6: Compound 3

To a stirred solution of 0.93 g (2.11 mmol) of 2-bromo-5-((4-(3-hydroxyphenyl)pyrimidin-2-yl)amino)phenol in 100 mL of N,N-dimethylformamide is added 1.46 g (10.59 mmol) of potassium carbonate then 0.49 g (2.11 mmol) of 1-bromo-2-(2-bromoethoxy)ethane in 50 mL of N,N-dimethylformamide for one hour. The reaction mixture is stirred at 75° C. for 20 hours. After returning to room temperature, the solvent is evaporated, water is added and the solid formed is filtered and dried under vacuum to afford 0.85 g (94%) of compound 3 as a beige powder.

LCMS (EI, m/z): (M+1) 429.27

¹H NMR: dH ppm (400 MHz, DMSO): 9.97 (1H, s, NH), 8.69 (1H, s, CHₐᵣₒₘ), 8.59-8.60 (1H, m, CHₐᵣₒₘ), 8.01 (1H, s, CHₐᵣₒₘ), 7.63-7.65 (1H, d, CHₐᵣₒₘ), 7.43-7.48 (3H, m, CHₐᵣₒₘ), 7.19-7.21 (1H, d, CHₐᵣₒₘ), 6.83-6.85 (1H, d, CHₐᵣₒₘ), 4.24-4.25 (4H, m, CH₂), 3.81-3.85 (4H, m, CH₂).

Compound 4:

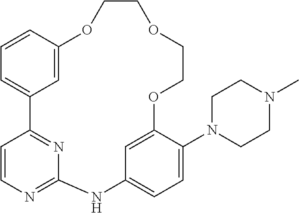 [SMILES: CN1CCN(c2ccc3cc2OCCOCCOc2cccc(c2)-c2ccnc(n2)N3)CC1]

In a 50 mL round-bottom flask are mixed 80 mg (0.168 mmol) of 2-(dicyclohexylphosphino)-2′,4′,6′-triisopropylbiphenyl, 308 mg (0.336 mmol) of (dppf)₂PdCl₂.CH₂Cl₂, 1.8 g (4.2 mmol) of compound 3 and 5.89 g (58 mmol) of 1-methylpiperazine under argon. 23 mL of tetrahydrofuran and 33 mL (33 mmol) of lithium bis(trimethylsilyl)amide (LiHMDS) are added at room temperature. The reaction mixture is heated to 85° C. for 5 hours. After returning to room temperature, the reaction is hydrolyzed by slow addition of water at 0° C. and the medium is extracted with ethyl acetate. The organic phase is dried over sodium sulfate, filtered and concentrated. The residue obtained is purified by chromatography on silica gel (eluent: ethyl acetate/methanol/ammonia: 94:4:2) to afford 692 mg (37%) of compound 4 as a yellow solid.

LCMS (EI, m/z): (M+1) 448.53

¹H NMR: dH ppm (400 MHz, DMSO): 9.63(1H, s, NH), 8.52-8.54 (1H, m, CHₐᵣₒₘ), 8.47 (1H, m, CHₐᵣₒₘ), 8.11 (1H, s, CHₐᵣₒₘ), 7.63-7.65 (1H, d, CHₐᵣₒₘ), 7.44-7.46 (1H, m, CHₐᵣₒₘ), 7.35-7.36 (1H, d, CHₐᵣₒₘ), 7.10-7.20 (1H, d, CHₐᵣₒₘ), 6.51-6.87 (2H, m, 4.21-4.27 (4H, m, CH₂), 3.84 (4H, m, CH₂), 3.67 (4H, m, CH₂), 2.46 (4H, m, CH₂), 2.22 (3H, m, CH₃).

Compound 5:

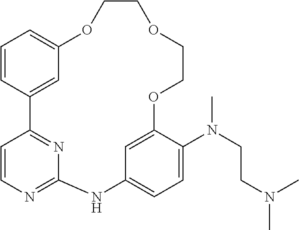 [SMILES: CN(C)CCN(C)c1ccc2cc1OCCOCCOc1cccc(c1)-c1ccnc(n1)N2]

Compound 5 was prepared according to the protocol described for the preparation of compound 3 starting with 150 mg of compound 3 and 107 mg of the amine N1,N1,N2-trimethylethane-1.2-diamine to afford 8.2 mg (5%) of compound 5.

LCMS (EI, m/z): (M+1) 450.54

¹H NMR: dH ppm (400 MHz, DMSO): 9.60 (1H, s, NH), 8.52-8.53 (1H, d, CHₐᵣₒₘ), 8.44 (1H, s, CHₐᵣₒₘ), 8.11 (1H, s, CHₐᵣₒₘ), 7.63-7.65 (1H, d, CHₐᵣₒₘ), 7.44-7.47 (1H, m, CHₐᵣₒₘ), 7.34-7.35 (1H, d, CHₐᵣₒₘ), 7.18-7.20 (1H, d, CHₐᵣₒₘ), 6.78-6.87 (2H, m, CHₐᵣₒₘ), 4.19-4.26 (4H, m, CH₂), 3.79-3.87 (4H, m, CH₂), 3.03-3.07 (2H, m, CH₂), 2.69 (3H, s, CH₂), 2.34-2.38 (2H, m, CH₂), 2.19 (6H, s, CH₂).

Compound 6:

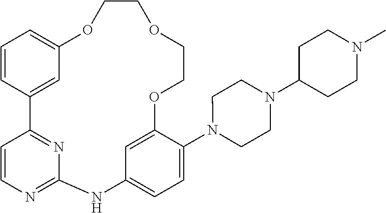 [SMILES: CN1CCC(N2CCN(c3ccc4cc3OCCOCCOc3cccc(c3)-c3ccnc(n3)N4)CC2)CC1]

Compound 6 was prepared according to the protocol described for the preparation of compound 3 starting with 150 mg of compound 3 and 193 mg of the amine 1-(1-methylpiperidin-4-yl)piperazine to afford 45 mg (23%) of compound 6.

LCMS (EI, m/z): (M+1) 431.66

¹H NMR: dH ppm (400 MHz, DMSO); 9.62 (1H, s, NH), 8.52-8.53 (1H, d, CHₐᵣₒₘ), 8.46 (1H, s, CHₐᵣₒₘ), 8.10 (1H, s, CHₐᵣₒₘ), 7.63-7.65 (1H, d, CHₐᵣₒₘ), 7.43-7.47 (1H, m, CHₐᵣₒₘ), 7.34-7.35 (1H, d, CHₐᵣₒₘ), 7.17-7.19 (1H, d, CHₐᵣₒₘ), 6.84 (2H, m, CHₐᵣₒₘ), 4.20-4.26 (4H, m, CH₂), 3.81-3.84 (4H, m, CH₂), 2.93 (4H, m, CH₂), 2.77-2.80 (2H, m, CH₂), 2.60 (4H, m, CH₂), 2.13 (4H, m, CH₂), 1.73-1.86 (4H, m, CH₂), 1.40-1.43 (2H, m, CH₂).

Compound 7:

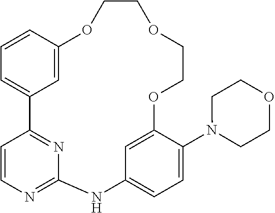 [SMILES: c1cc2cc(c1)-c1ccnc(n1)Nc1ccc(N3CCOCC3)c(c1)OCCOCCO2]

Compound 7 was prepared according to the protocol described for the preparation of compound 3 starting with 150 mg of compound 3 and 92 mg of the amine morpholine to afford 25 mg (16%) of compound 7.

LCMS (EI, m/z): (M+1) 435.48

¹H NMR: dH ppm (400 MHz, DMSO): 9.97 (1H, s, NH), 8.48-8.54 (2H, m, CHₐᵣₒₘ), 8.10 (1H, s, CHₐᵣₒₘ), 7.63-7.65 (1H, d, CHₐᵣₒₘ), 7.45 (1H, t, CHₐᵣₒₘ), 7.35-7.36 (1H, d, CHₐᵣₒₘ), 7.17-7.19 (1H, d, CHₐᵣₒₘ), 6.84 (2H, m, CHₐᵣₒₘ), 4.25 (4H, m, CH₂), 3.84 (4H, m, CH₂), 3.72-3.80 (4H, m, CH₂), 2.93 (4H, m, CH₂).

Compound 8:

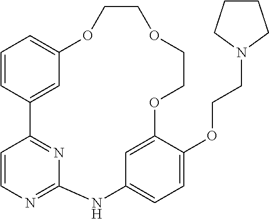 [SMILES: c1cc2cc(c1)-c1ccnc(n1)Nc1ccc(OCCN3CCCC3)c(c1)OCCOCCO2]

In a microwave reactor are mixed 150 mg (3.5 mmol) of compound 3, 66.7 mg (0.36 mmol) of cuprous iodide and 0.4 mL of (hydroxyethyl)pyrrolidine (5.13 mmol). The reaction mixture is heated to 200° C. for 30 minutes. After returning to room temperature, the reaction is hydrolyzed by addition of water and extracted with ethyl acetate. The organic phase is dried over sodium sulfate, filtered and concentrated. The residue obtained is purified by chromatography on silica gel (eluent: ethyl acetate/methanol/ammonia: 94:4:2) to afford 24 mg (15%) of compound 8 as a yellow solid.

LCMS (EI, m/z): (M+1) 463.54

¹H NMR: dH ppm (400 MHz, DMSO): 9.64 (1H, s, NH), 8.52-8.54 (1H, d, CHₐᵣₒₘ), 8.49 (1H, s, CHₐᵣₒₘ), 8.09 (1H, s, CHₐᵣₒₘ), 7.63-7.65 (1H, d, CHₐᵣₒₘ), 7.43-7.47 (1H, m, CHₐᵣₒₘ), 7.35-7.36 (1H, d, CHₐᵣₒₘ), 7.16-7.18 (1H, d, CHₐᵣₒₘ) 6.94-6.97 (1H, m, CHₐᵣₒₘ), 6.82-6.84 (1H, m, CHₐᵣₒₘ), 4.20-4.27 (4H, m, CH₂), 4.03-4.04 (2H, m, CH₂), 3.80 (4H, m, CH₂), 2.68 (2H, s, CH₂), 2.50 (4H, m, CH₂), 1.68 (4H, s, CH₂).

Compound 9:

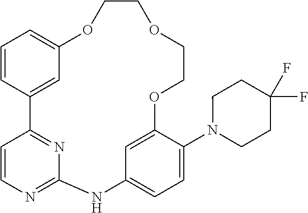 [SMILES: FC1(F)CCN(c2ccc3cc2OCCOCCOc2cccc(c2)-c2ccnc(n2)N3)CC1]

Compound 9 was prepared according to the protocol described for the preparation of compound 3 starting with 200 mg of compound 3 and 294 mg of the amine 4,4-difluoropiperidine to afford 26 mg (12%) of compound 9.

LCMS (EI, m/z): (M+1) 469.50

¹H NMR: dH ppm (400 MHz, DMSO): 9.68 (1H, s, NH), 8.53-8.55 (1H, d, CHₐᵣₒₘ), 8.50 (1H, s, CHₐᵣₒₘ), 8.11 (1H, s, CHₐᵣₒₘ), 7.64-7.66 (1H, d, CHₐᵣₒₘ), 7.44-7.48 (1H, m, CHₐᵣₒₘ), 7.36-7.38 (1H, d, CHₐᵣₒₘ), 7.18-7.20 (1H, d, CHₐᵣₒₘ), 6.91-6.93 (1H, m, CHₐᵣₒₘ), 6.81-6.83 (1H, m, CHₐᵣₒₘ), 4.24-4.25 (4H, m, CH₂), 3.80-3.88 (4H, m, CH₂), 3.06 (4H, m, CH₂), 2.10 (4H, m, CH₂), 2.50 (4H, m, CH₂).

Compound 10:

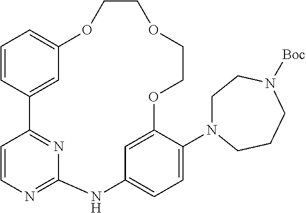 [SMILES: CC(C)(C)OC(=O)N1CCCN(c2ccc3cc2OCCOCCOc2cccc(c2)-c2ccnc(n2)N3)CC1]

Compound 10 was prepared according to the protocol described for the preparation of compound 3 starting with 400 mg of compound 3 and 561 mg of the amine tert-butyl 1,4-diazepane-1-carboxylate to afford 90 mg (18%) of compound 10.

LCMS (EI, m/z): (M+1) 548.64

Compound 11:

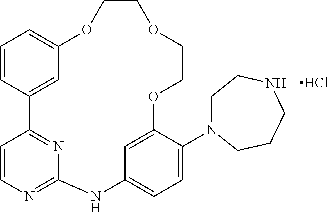 [SMILES: c1cc2cc(c1)-c1ccnc(n1)Nc1ccc(N3CCCNCC3)c(c1)OCCOCCO2 ; Cl]

To 90 mg (0.16 mmol) of compound 10 is added dropwise 10 mL of a solution of hydrochloric acid in isopropanol (5N). The solution is stirred at 20° C. for 8 h. After evaporation to dryness, the solid formed is triturated in ether then recrystallized in isopropanol to obtain 80 mg (92%) of compound 11 as a yellow powder.

LCMS (EI, m/z): (M+1) 448.53

¹H NMR: dH ppm (400 MHz, DMSO): 9.71 (1H, s, NH), 8.88 (1H, s, CHₐᵣₒₘ), 8.53-8.55 (1H, d, CHₐᵣₒₘ), 8.49 (1H, s, CHₐᵣₒₘ), 8.10 (1H, s, CHₐᵣₒₘ), 7.63-7.66 (1H, d, CHₐᵣₒₘ), 7.44-7.48 (1H, t, CHₐᵣₒₘ), 7.37-7.38 (1H, d, CHₐᵣₒₘ), 7.18-7.20 (1H, dd, CHₐᵣₒₘ), 6.78-6.80 (1H, d, CHₐᵣₒₘ), 4.25 (4H, m, CH₂), 3.25-3.26 (6H, m, CH₂), 3.06 (4H, m, CH₂), 2.50 (4H, m, CH₂).

Compound 12:

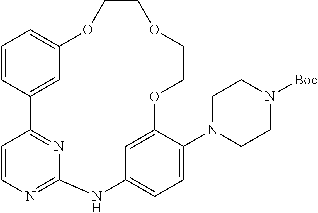 [SMILES: CC(C)(C)OC(=O)N1CCN(c2ccc3cc2OCCOCCOc2cccc(c2)-c2ccnc(n2)N3)CC1]

Compound 12 was prepared according to the protocol described for the preparation of compound 3 starting with 600 mg of compound 3 and 2.08 g of the amine tert-butyl piperazine-1-carboxylate to afford 312 mg (42%) of compound 12.

LCMS (EI, m/z): (M+1) 534.62

¹H NMR: dH ppm (400 MHz, DMSO): 9.65 (1H, s, NH), 8.48-8.53 (2H, m, CHₐᵣₒₘ), 8.10-8.11 (1H, d, CHₐᵣₒₘ), 7.62-7.64 (1H, d, CHₐᵣₒₘ), 7.43-7.47 (1H, t, CHₐᵣₒₘ), 7.35-7.36 (1H, d, CHₐᵣₒₘ), 7.18-7.20 (1H, d, CHₐᵣₒₘ), 6.82-6.84 (2H, m, CHₐᵣₒₘ), 4.21-4.26 (4H, m, CH₂), 3.80-3.86 (4H, m, CH₂), 3.44 (4H, m, CH₂), 2.28 (4H, m, CH₂), 1.42 (9H, s, CH₃).

Compound 13:

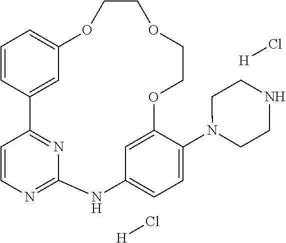

To 312 mg (0.58 mmol) of compound 12 is added dropwise 3 mL of a solution of hydrochloric acid in isopropanol (5N). The solution is stirred at 45° C. for 1 h. After evaporation to dryness, the solid formed is triturated in ether then recrystallized in isopropanol to obtain 296 mg (95%) of compound 13 as a yellow powder.

LCMS (EI, m/z): (M+1) 434.21

¹H NMR: dH ppm (400 MHz, DMSO): 9.98 (1H, s, NH), 9.45 (1H, s, NH), 8.57-8.58 (1H, d, CHₐᵣₒₘ), 8.51-8.52 (1H, m, CHₐᵣₒₘ), 8.09 (1H, t, CHₐᵣₒₘ), 7.66-7.68 (1H, d, CHₐᵣₒₘ), 7.43-7.48 (2H, m, CHₐᵣₒₘ), 7.19-7.21 (1H, dd, CHₐᵣₒₘ), 7.11-7.12 (1H, d, CHₐᵣₒₘ), 6.85-6.88 (1H, d, CHₐᵣₒₘ), 4.24-4.27 (4H, m, CH₂), 3.88-3.91 (2H, m, CH₂), 3.78-3.81 (2H, m, CH₂), 3.31 (8H, m, CH₂).

Compound 14:

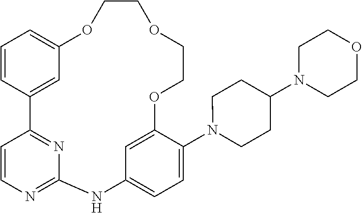 [SMILES: c1cc2cc(c1)-c1ccnc(n1)Nc1ccc(N3CCC(N4CCOCC4)CC3)c(c1)OCCOCCO2]

Compound 14 was prepared according to the protocol described for the preparation of compound 3 starting with 400 mg of compound 3 and 159 mg of the amine 4-(piperidine-4-yl)morpholine to afford 58 mg (12%) of compound 14.

LCMS (EI, m/z): (M+1) 518.61

¹H NMR: dH ppm (400 MHz, DMSO): 9.60 (1H, s, NH), 8.51-8.52 (1H, d, CHₐᵣₒₘ), 8.45-8.46 (1H, d, CHₐᵣₒₘ), 8.10 (1H, s, CHₐᵣₒₘ), 7.62-7.64 (1H, d, CHₐᵣₒₘ), 7.43-7.47 (1H, t, CHₐᵣₒₘ), 7.33-7.35 (1H, d, CHₐᵣₒₘ), 7.18-7.20 (1H, d, CHₐᵣₒₘ), 6.81-6.83 (2H, m, CHₐᵣₒₘ), 4.21-4.26 (4H, m, CH₂), 3.79-3.85 (4H, m, CH₂), 3.85 (4H, m, CH₂), 3.33 (8H, m, CH₂), 2.17-2.24 (1H, t, CH₂), 1.83-1.86 (2H, d, CH₂), 1.47-1.56 (2H, d, CH₂).

Compound 15:

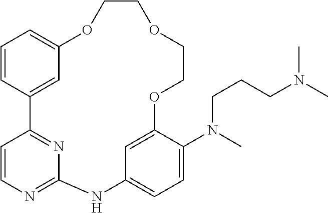 [SMILES: CN(C)CCCN(C)c1ccc2cc1OCCOCCOc1cccc(c1)-c1ccnc(n1)N2]

Compound 15 was prepared according to the protocol described for the preparation of compound 3 starting with 400 mg of compound 3 and 109 mg of the amine N1,N1,N3-trimethyl-propane-1,3-diamine to afford 53 mg (12%) of compound 15.

LCMS (EI, m/z): (M+1) 464.57

¹H NMR: dH ppm (400 MHz, DMSO): 9.58 (1H, s, NH), 8.51-8.52 (1H, d, CHₐᵣₒₘ), 8.43 (1H, s, CHₐᵣₒₘ), 8.10 (1H, s, CHₐᵣₒₘ), 7.62-7.64 (1H, d, CHₐᵣₒₘ), 7.42-7.46 (1H, t, CHₐᵣₒₘ), 7.33-7.34 (1H, d, CHₐᵣₒₘ), 7.16-7.18 (1H, d, CHₐᵣₒₘ), 6.78-6.86 (2H, m, CHₐᵣₒₘ), 4.18-4.25 (4H, m, CH₂), 3.80-3.86 (4H, m, CH₂), 2.93-2.96 (2H, m, CH₂), 2.21-2.22 (2H, t, CH₂), 2.09 (9H, s, CH₃), 1.55-1.58 (2H, m, CH₃).

Compound 16:

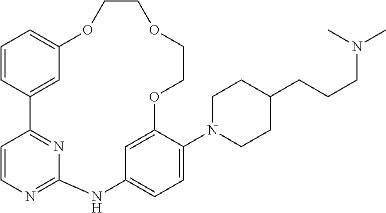 [SMILES: CN(C)CCCC1CCN(c2ccc3cc2OCCOCCOc2cccc(c2)-c2ccnc(n2)N3)CC1]

Compound 16 was prepared according to the protocol described for the preparation of compound 3 starting with 400 mg of compound 3 and 160 mg of the amine 4-[3-(N,N-dimethylamino)-propyl]-piperazine to afford 90 mg (19%) of compound 16.

LCMS (EI, m/z): (M+1) 519.65

¹H NMR: dH ppm (400 MHz, DMSO): 9.61 (1H, s, NH), 8.51-8.52 (1H, d, CHₐᵣₒₘ), 8.46-8.47 (1H, d, CHₐᵣₒₘ), 8.09-8.10 (1H, t, CHₐᵣₒₘ), 7.62-7.64 (1H, d, CHₐᵣₒₘ), 7.42-7.46 (1H, t, CHₐᵣₒₘ), 7.33-7.35 (1H, d, CHₐᵣₒₘ), 7.16-7.18 (1H, dd, CHₐᵣₒₘ), 6.82-6.85 (2H, m, CHₐᵣₒₘ), 4.20-4.26 (4H, m, CH₂), 3.79-3.83 (4H, m, CH₂), 2.29 (4H, m, CH₂), 2.30-2.33 (2H, t, CH₂), 2.49 (4H, m, CH₂), 2.19-2.23 (2H, d, CH₂), 2.11 (6H, s, CH₃), 1.54-1.60 (2H, m, CH₂).

Compound 17:

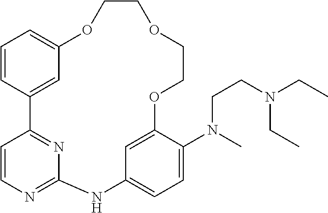 [SMILES: CCN(CC)CCN(C)c1ccc2cc1OCCOCCOc1cccc(c1)-c1ccnc(n1)N2]

Compound 17 was prepared according to the protocol described for the preparation of compound 3 starting with 400 mg of compound 3 and 122 mg of the amine N1,N1-diethyl-N2-methyl-ethane-1,2-diamine to afford 53 mg (12%) of compound 17.

LCMS (EI, m/z): (M+1) 478.59

¹H NMR: dH ppm (400 MHz, DMSO): 9.66 (1H, s, NH), 8.53-8.54 (1H, d, CHₐᵣₒₘ), 8.46-8.47 (1H, d, CHₐᵣₒₘ), 8.09-8.10 (1H, t, CHₐᵣₒₘ), 7.62-7.64 (1H, d, CHₐᵣₒₘ), 7.43-7.47 (1H, t, CHₐᵣₒₘ), 7.35-7.36 (1H, d, CHₐᵣₒₘ), 7.17-7.20 (1H, dd, CHₐᵣₒₘ), 6.96-6.98 (1H, m, CHₐᵣₒₘ), 6.80-6.82 (1H, dd, CHₐᵣₒₘ), 4.16-4.25 (4H, m, CH₂), 3.87-3.90 (2H, t, CH₂), 3.79-3.81 (2H, m, CH₂), 3.13-3.32 (8H, t, CH₂), 2.27 (3H, s, CH₃), 1.11-1.20 (6H, m, CH₃).

Compound 18:

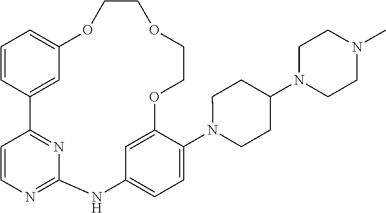 [SMILES: CN1CCN(C2CCN(c3ccc4cc3OCCOCCOc3cccc(c3)-c3ccnc(n3)N4)CC2)CC1]

Compound 18 was prepared according to the protocol described for the preparation of compound 3 starting with 400 mg of compound 3 and 171 mg of the amine 1-methyl-4-(piperidine-4-yl)piperazine to afford 63 mg (12%) of compound 18.

LCMS (EI, m/z): (M+1) 531.30

¹H NMR: dH ppm (400 MHz, DMSO): 9.60 (1H, s, NH), 8.51-8.53 (1H, d, CHₐᵣₒₘ), 8.45 (1H, s, CHₐᵣₒₘ), 8.10 (1H, s, CHₐᵣₒₘ), 7.62-7.64 (1H, d, CHₐᵣₒₘ), 7.42-7.47 (1H, t, CHₐᵣₒₘ), 7.33-7.34 (1H, d, CHₐᵣₒₘ), 7.16-7.18 (1H, dd, Cₐᵣₒₘ), 6.79-6.85 (2H, m, CHₐᵣₒₘ), 4.20-4.25 (4H, m, CH₂), 3.80-3.84 (4H, m, CH₂), 3.32-3.37 (4H, m, CH₂), 2.20-2.32 (8H, t, CH₂), 2.20-2.27 (1H, t, CH₂), 2.12 (3H, s, CH₃), 1.81-1.83 (2H, m, CH₂), 1.51-1.53 (2H, m, CH₂).

Compound 19:

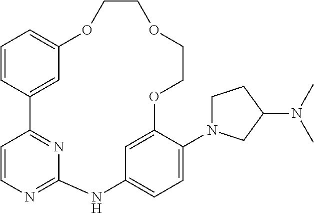 [SMILES: CN(C)C1CCN(c2ccc3cc2OCCOCCOc2cccc(c2)-c2ccnc(n2)N3)C1]

Compound 19 was prepared according to the protocol described for the preparation of compound 3 starting with 400 mg of compound 3 and 107 mg of the amine N,N-dimethyl-pyrrolidin-3-diamine to afford 28 mg (6%) of compound 19.

LCMS (EI, m/z): (M+1) 462.24

¹H NMR: dH ppm (400 MHz, DMSO): 9.51 (1H, s, NH), 8.49-8.50 (1H, d, CHₐᵣₒₘ), 8.41 (1H, s, CHₐᵣₒₘ), 8.10 (1H, s, CHₐᵣₒₘ), 7.61-7.63 (1H, d, CHₐᵣₒₘ), 7.42-7.46 (1H, t, CHₐᵣₒₘ), 7.30-7.31 (1H, d, CHₐᵣₒₘ), 7.16-7.18 (1H, d, CHₐᵣₒₘ), 6.68-6.78 (1H, m, CHₐᵣₒₘ), 6.65-6.67 (1H, m, CHₐᵣₒₘ), 4.09-4.24 (4H, m, CH₂), 3.79-3.85 (4H, m, CH₂), 3.19-3.21 (2H, m, CH₂), 3.11-3.15 (1H, t, CH₂), 2.22 (6H, s, CH₃), 1.99-2.07 (2H, m, CH₂), 1.68-1.77 (2H, m, CH₂).

Compound 20:

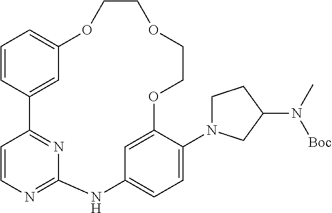 [SMILES: CN(C(=O)OC(C)(C)C)C1CCN(c2ccc3cc2OCCOCCOc2cccc(c2)-c2ccnc(n2)N3)C1]

Compound 20 was prepared according to the protocol described for the preparation of compound 3 starting with 200 mg of compound 3 and 374 mg of the amine tert-butyl methyl(pyrrolidin-3-yl)carbamate to afford 60 mg (22%) of compound 20.

LCMS (EI, m/z): (M+1) 548.64

Compound 21:

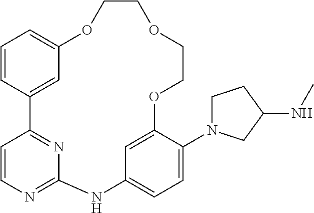 [SMILES: CNC1CCN(c2ccc3cc2OCCOCCOc2cccc(c2)-c2ccnc(n2)N3)C1]

To 60 mg (0.11 mmol) of compound 20 in 2 mL of dichloromethane is added 25 μL (0.329 mmol) of trifluoroacetic acid (TFA) at 0° C. in small portions. The reaction mixture is stirred at 25° C. for 12 hours. The solvent is evaporated, the reaction is hydrolyzed by addition of water, basified and extracted with ethyl acetate. The organic phase is dried over sodium sulfate, filtered and concentrated. The residue obtained is purified by chromatography on silica gel (eluent: ethyl acetate/methanol/ammonia: 90:8:2) to afford 24 mg (48%) of compound 21 as a yellow solid.

LCMS (EI, m/z): (M+1) 448.53

¹H NMR: dH ppm (400 MHz, DMSO): 9.51 (1H, s, NH), 8.50-8.51 (1H, d, CHₐᵣₒₘ), 8.41-8.42 (1H, d, CHₐᵣₒₘ), 8.11 (1H, s, CHₐᵣₒₘ), 7.62-7.64 (1H, d, CHₐᵣₒₘ), 7.43-7.47 (1H, t, CHₐᵣₒₘ), 7.30-7.31 (1H, d, CHₐᵣₒₘ), 7.17-7.19 (1H, d, CHₐᵣₒₘ), 6.77-6.79 (1H, m, CHₐᵣₒₘ), 6.63-6.66 (1H, m, CHₐᵣₒₘ), 4.25-4.27 (2H, m, CH₂), 4.13-4.14 (2H, m, CH₂), 3.79-3.86 (4H, m, CH₂), 3.20 (2H, m, CH₂), 3.10-3.21 (3H, m, NH and CH₂), 3.01-3.03 (1H, m, CH), 2.31 (3H, s, CH₃), 2.01-2.05 (1H, m, CH), 1.64-1.68 (1H, m, CH).

Compound 22:

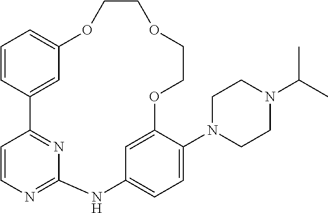 [SMILES: CC(C)N1CCN(c2ccc3cc2OCCOCCOc2cccc(c2)-c2ccnc(n2)N3)CC1]

To a solution of 120 mg (0.24 mmol) of compound 13 and 41 mg (0.71 mmol) of propan-2-one in 3 mL of 1,2-dichloroethane are added 68 μL of acetic acid (1.18 mmol) and, in small fractions, 201 mg (0.95 mmol) of sodium triacetoxyborohydride. The reaction mixture is stirred for 16 hours at room temperature. The solvent is then concentrated, the reaction mixture extracted with ethyl acetate and washed using saturated sodium hydroxide solution. The organic phases are combined, dried over magnesium sulfate then concentrated. The residue is purified by chromatography on silica gel (eluent: dichloromethane/methanol/ammonia: 95:4:1) to afford 24 mg (21%) of compound 22 as a yellow solid,

LCMS (EI, m/z): (M+1) 476.58

¹H NMR: dH ppm (400 MHz, CDCl₃): 8.58-8.60 (1H, d, CHₐᵣₒₘ), 8.42-8.43 (1H, d, CHₐᵣₒₘ), 8.23-8.24 (1H, t, CHₐᵣₒₘ), 8.44-8.46 (1H, d, CHₐᵣₒₘ), 7.36-7.39 (1H, t, CHₐᵣₒₘ), 7.20 (1H, s, NH), 7.12-7.14 (1H, d, CHₐᵣₒₘ), 7.05-7.07 (1H, dd, CHₐᵣₒₘ), 6.88-6.90 (1H, d, CHₐᵣₒₘ), 6.51-6.54 (1H, dt, CHₐᵣₒₘ) 4.39-4.42 (2H, t, CH₂), 4.29-4.31 (2H, m, CH₂), 3.96-4.00 (2H, m, CH₂), 3.89-3.91 (2H, m, CH₂), 2.62-3.22 (8H, m, CH₂), 2.49-2.58 (1H, m, CH), 1.17 (6H, s, CH₃).

Compound 23:

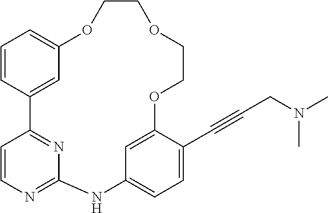 [SMILES: CN(C)CC#Cc1ccc2cc1OCCOCCOc1cccc(c1)-c1ccnc(n1)N2]

In a 10 mL vial are mixed 107 mg (0.25 mmol) of compound 3, 27 mg (0.058 mmol) of 2-(dicyclohexylphosphino)-2′,4′,6′-triisopropylbiphenyl, 4.79 mg (0.013 mmol) of benzonitrile palladium(II) chloride, 48 μL (0.450 mmol) of dimethylamino-2-propyne and 244 mg (0.75 mmol) of cesium carbonate in 0.3 mL of dimethylformamide at room temperature. 81 mg (0.250 mmol) of tetrabutylammonium bromide is added to the reaction mixture. The reaction mixture is heated to 80° C. for 15 hours. After returning to room temperature, the reaction is hydrolyzed by slow addition of water and extracted with ethyl acetate. The organic phase is dried over sodium sulfate, filtered and concentrated. The residue obtained is purified by chromatography on silica gel (eluent: ethyl acetate/methanol/ammonia: 95:4:1) to afford 9 mg (9%) of compound 23 as a yellow solid.

LCMS (EI, m/z): (M+1) 431.50

¹H NMR: dH ppm (400 MHz, DMSO): 10.00 (1H, s, NH), 8.63-8.64 (1H, d, CHₐᵣₒₘ), 8.59-8.60 (1H, d, CHₐᵣₒₘ), 8.11 (1H, m, CHₐᵣₒₘ), 7.64-7.65 (1H, d, CHₐᵣₒₘ), 7.44-7.48 (2H, m, CHₐᵣₒₘ), 7.23-7.25 (1H, d, CHₐᵣₒₘ), 7.18-7.20 (1H, d, CHₐᵣₒₘ), 6.83-6.85 (1H, dd, CHₐᵣₒₘ), 4.23-4.28 (4H, m, CH₂), 3.80-3.86 (4H, m, CH₂), 3.42 (2H, m, CH₂), 2.24 (6H, s, CH₃).

Compound 24:

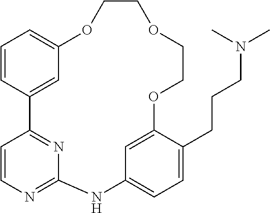 [SMILES: CN(C)CCCc1ccc2cc1OCCOCCOc1cccc(c1)-c1ccnc(n1)N2]

In a round-bottom flask and under argon, introduce 40 mg (0.09 mmol) of compound 23 in 10 mL of a mixture of THF/MeOH (1:1). Degas the mixture under argon and under vacuum. Add 9.8 mg (0.009 mmol) of Pd—C. Degas the mixture under argon and under vacuum then place in a round-bottom flask of hydrogen. The reaction mixture is stirred at 25° C. overnight then filtered on silica and rinsed with ethyl acetate then concentrated. The residue obtained is purified by chromatography on silica gel (eluent: ethyl acetate/methanol/ammonia: 90:8:2) to afford 5.1 mg (12%) of compound 24 as a yellow solid.

LCMS (EI, m/z): (M+1) 435.53

¹H NMR: dH ppm (400 MHz, DMSO): 9.70 (1H, s, NH), 8.54-8.55 (1H, d, CHₐᵣₒₘ), 8.47-8.48 (1H, d, CHₐᵣₒₘ), 8.12 (1H, s, CHₐᵣₒₘ), 7.63-7.64 (1H, d, CHₐᵣₒₘ), 7.44-7.48 (1H, t, CHₐᵣₒₘ), 7.37-7.38 (1H, d, CHₐᵣₒₘ), 7.17-7.20 (1H, d, CHₐᵣₒₘ), 7.00-7.03 (1H, dd, CHₐᵣₒₘ), 6.76-6.78 (1H, dd, CHₐᵣₒₘ), 4.10-4.24 (4H, m, CH₂), 3.79-3.87 (4H, m, CH₂), 2.18-2.21 (2H, m, CH₂), 2.45 (2H, m, CH₂), 2.11 (6H, s, CH₃), 1.60-1.64 (2H, m, CH₂).

Compound 25:

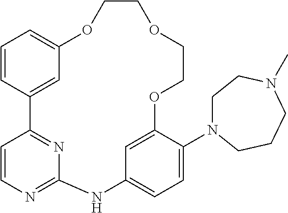 [SMILES: CN1CCCN(c2ccc3cc2OCCOCCOc2cccc(c2)-c2ccnc(n2)N3)CC1]

To a solution of 70 mg (0.13 mmol) of compound 11 in 2 mL of 1,2-dichloroethane is added 37.5 μL (0.269 mmol) of triethylamine. Stir for 10 minutes then add 29.3 μL (0.403 mmol) of aqueous formaldehyde solution and 38.5 μL (0.672 mmol) of acetic acid, then 114 mg (0.538 mmol) of sodium triacetoxyborohydride. The reaction mixture is stirred for 24 hours at room temperature. The solvent is then concentrated, the reaction mixture extracted with ethyl acetate and washed using 1N saturated sodium hydroxide solution. The organic phases are combined, dried over magnesium sulfate then concentrated. The residue is purified by chromatography on silica gel (eluent: dichloromethane/methanol: 90:10) to afford 29 mg (43%) of compound 25 as a yellow solid,

LCMS (EI, m/z): (M+1) 462.56

¹H NMR: dH ppm (400 MHz, DMSO): 9.57 (1H, s, NH), 8.50-8.81 (1H, s, CHₐᵣₒₘ), 8.42-8.43 (1H, d, CHₐᵣₒₘ), 8.10 (1H, dd, CHₐᵣₒₘ), 7.62-7.64 (1H, d, CHₐᵣₒₘ), 7.43-7.47 (1H, dd, CHₐᵣₒₘ), 7.32-7.35 (1H, d, CHₐᵣₒₘ), 7.16-7.17 (1H, dd, CHₐᵣₒₘ), 6.74-6.83 (2H, d, CHₐᵣₒₘ), 4.17-4.25 (4H, m, CH₂), 3.79-3.84 (4H, m, CH₂), 3.16-3.21 (4H, m, CH₂), 2.55-2.67 (4H, m, CH₂), 2.28 (3H, m, CH₃), 1.83-1.90 (2H, m, CH₂).

Compound 26:

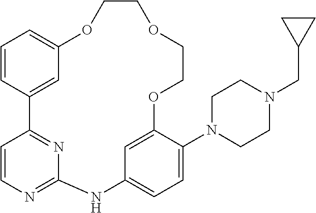 [SMILES: c1cc2cc(c1)-c1ccnc(n1)Nc1ccc(N3CCN(CC4CC4)CC3)c(c1)OCCOCCO2]

To a solution of 120 mg (0.13 mmol) of compound 13 in 3 mL of 1,2-dichloroethane is added 61 μL (0.47 mmol) of triethylamine. Stir for 10 minutes then add 53 μL (0.71 mmol) of cyclopropane carbaldehyde and 136 μL (2.37 mmol) of acetic acid, then 201 mg (0.94 mmol) of sodium triacetoxyborohydride. The reaction mixture is stirred for 24 hours at room temperature. The solvent is then concentrated, the reaction mixture extracted with ethyl acetate and washed using 1N saturated sodium hydroxide solution. The organic phases are combined, dried over magnesium sulfate then concentrated. The residue is purified by chromatography on silica gel (eluent: dichloromethane/methanol/ammonia: 90:8:2) to afford 28 mg (24%) of compound 26 as a yellow solid.

LCMS (EI, m/z): (M+1) 488.59

¹H NMR: dH ppm (400 MHz, DMSO): 9.62 (1H, d, CHₐᵣₒₘ), 8.51-8.53 (1H, d, CHₐᵣₒₘ), 8.46-8.47 (1H, d, CHₐᵣₒₘ), 8.10 (1H, t, CHₐᵣₒₘ), 7.62-7.64 (1H, d, CHₐᵣₒₘ), 7.42-7.46 (1H, t, CHₐᵣₒₘ), 7.34-7.35 (1H, d, CHₐᵣₒₘ), 7.16-7.18 (1H, dd, CHₐᵣₒₘ), 6.80-6.86 (2H, m, CHₐᵣₒₘ), 4.20-4.26 (4H, m, CH₂), 3.78-3.86 (4H, m, CH₂), 2.94 (4H, m, CH₂), 2.56 (4H, m, CH₂), 2.22 (2H, m, CH), 0.85 (1H, s, CH), 0.47 (2H, m, CH₂), 0.09 (2H, s, CH₂).

Compound 27:

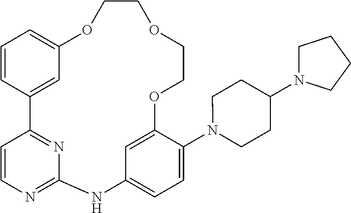 [SMILES: c1cc2cc(c1)-c1ccnc(n1)Nc1ccc(N3CCC(N4CCCC4)CC3)c(c1)OCCOCCO2]

Compound 27 was prepared according to the protocol described for the preparation of compound 3 starting with 400 mg of compound 3 and 577 mg of the amine 4-(pyrrolidin-1-yl)piperidine to afford 63 mg (13%) of compound 27.

LCMS (EI, m/z): (M+1) 502.62

¹H NMR: dH ppm (400 MHz, DMSO): 9.61 (1H, s, NH), 8.51-8.52 (1H, d, CHₐᵣₒₘ), 8.45-8.46 (1H, d, CHₐᵣₒₘ), 8.10 (1H, s, CHₐᵣₒₘ), 7.62-7.64 (1H, d, CHₐᵣₒₘ), 7.42-7.46 (1H, t, CHₐᵣₒₘ), 7.33-7.35 (1H, d, CHₐᵣₒₘ), 7.16-7.18 (1H, dd, CHₐᵣₒₘ), 6.79-6.85 (2H, m, CHₐᵣₒₘ), 4.21-4.26 (4H, m, CH₂), 3.79-3.84 (4H, m, CH₂), 3.27-3.32 (4H, m, CH₂), 2.25-2.58 (5H, m, CH and CH₂), 1.89-1.94 (2H, m, CH₂), 1.68 (4H, m, CH₂), 1.46-1.57 (2H, m, CH₂).

Compound 28:

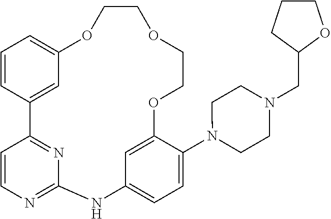 [SMILES: c1cc2cc(c1)-c1ccnc(n1)Nc1ccc(N3CCN(CC4CCCO4)CC3)c(c1)OCCOCCO2]

Compound 28 was prepared according to the protocol described for the preparation of compound 3 starting with 400 mg of compound 3 and 637 mg of the amine 1-((tetrahydrofuran-2-yl)methyl)piperazine to afford 108 mg (22%) of compound 28.

LCMS (EI, m/z): (M+1) 518.62

¹H NMR: dH ppm (400 MHz, DMSO): 9.62 (1H, s, NH), 8.51-8.52 (1H, d, CHₐᵣₒₘ), 8.45-8.46 (1H, d, CHₐᵣₒₘ), 8.10 (1H, s, CHₐᵣₒₘ), 7.62-7.64 (1H, d, CHₐᵣₒₘ), 7.42-7.46 (1H, t, CHₐᵣₒₘ), 7.34-7.35 (1H, d, CHₐᵣₒₘ), 7.16-7.18 (1H, dd, CHₐᵣₒₘ), 6.80-6.85 (2H, m, CHₐᵣₒₘ), 4.20-4.26 (4H, m, CH₂), 3.90-3.97 (1H, m, CH), 3.78-3.84 (4H, m, CH₂), 3.71-3.77 (1H, m, CH), 3.58-3.62 (1H, m, CH), 2.92 (4H, m, CH₂), 2.47-2.67 (4H, m, CH₂), 2.38-2.42 (2H, m, CH₂), 1.89-1.97 (1H, m, CH), 1.73-1.83 (2H, m, CH₂), 1.44-1.53 (1H, m, CH).

Compound 29:

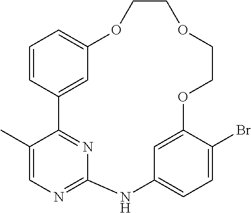 [SMILES: Cc1cnc2nc1-c1cccc(c1)OCCOCCOc1cc(ccc1Br)N2]

Step 1: Intermediate 10

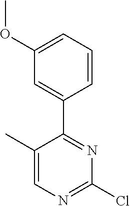 [SMILES: COc1cccc(-c2nc(Cl)ncc2C)c1]

To 1.12 g (6.91 mmol) of 2,4-dichloro-5-methylpyrimidine in 50 mL of anhydrous THF is added 1 g (6.58 mmol) of 3-methoxyphenylboronic acid. The reaction mixture is stirred at room temperature for 10 minutes, then 1.74 g (16.45 mmol) of sodium carbonate dissolved in 8 mL of water and then 0.38 g (0.329 mmol) of tetrakis(triphenylphosphine)palladium(0) are added at room temperature. The reaction mixture is stirred at 90° C. for 16 hours. Ethyl acetate is added and the organic phase is washed with saturated sodium chloride solution, dried over magnesium sulfate, filtered. The filtrate is evaporated and the residue purified by silica column chromatography using Companion® (eluent: cyclohexane/ethyl acetate: 0 to 10%, then cyclohexane/ethyl acetate: 95:5) to afford 1.2 g (77%) of intermediate 10 as a white solid.

LCMS (EI, m/z): (M+1) 235.68

¹H NMR: dH ppm (400 MHz, DMSO): 8.72 (1H, s, CHₐᵣₒₘ), 7.44-7.48 (1H, d, CHₐᵣₒₘ), 7.21-7.23 (1H, d, CHₐᵣₒₘ), 7.18 (1H, m, CHₐᵣₒₘ), 7.10-7.13 (1H, dd, CHₐᵣₒₘ), 3.83 (3H, s, CH₃), 3.33 (3H, s, CH₃).

Step 2: Intermediate 11

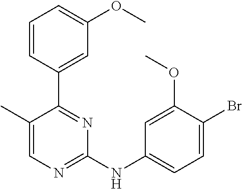 [SMILES: COc1cccc(-c2nc(Nc3ccc(Br)c(OC)c3)ncc2C)c1]

To a solution of 15 mL of n-butanol comprising 0.6 g (2.56 mmol) of intermediate 10 and 0.52 g (2.56 mmol) of 4-bromo-3-methoxyaniline is added 0.5 mL of hydrochloric acid solution (5N). The reaction mixture is stirred at 80° C. overnight. After returning to room temperature, the solid formed is filtered, rinsed with n-butanol to afford 0.79 g (78%) of intermediate 11 as a yellow powder.

LCMS (EI, m/z): (M+1) 401.26

¹H NMR: dH ppm (400 MHz, DMSO): 9.76 (1H, s, NH), 8.46 (1H, d, CHₐᵣₒₘ), 7.92 (1H, s, CHₐᵣₒₘ), 7.38-7.46 (2H, m, CHₐᵣₒₘ), 7.24-7.28 (3H, m, CHₐᵣₒₘ), 7.06-7.08 (1H, d, CHₐᵣₒₘ), 3.81 (3H, s, CH₃), 3.87 (3H, s, CH₃), 2.24 (3H, s, CH₃).

Step 3: Intermediate 12

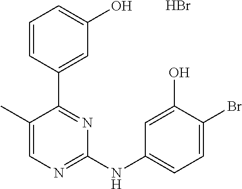 [SMILES: Br ; Cc1cnc(Nc2ccc(Br)c(O)c2)nc1-c1cccc(O)c1]

To a solution of 0.25 g (0.62 mmol) of intermediate 11 in 7 mL of dichloromethane is added 0.3 mL (3.12 mmol) of tribromo-borane at −78° C. The reaction mixture is then stirred at 45° C. for 5 h then overnight at room temperature. 20 mL of methanol is added at 0° C. to the reaction mixture which is then heated to 35° C. for 25 minutes. The solid formed is filtered then washed twice with 20 mL of ether to afford 0.27 g (99%) of intermediate 12 as a yellow powder.

LC-MS (EI m/z): (M+H⁺): 372.02+374.02

¹H NMR: dH ppm (400 MHz, DMSO): 9.62 (1H, s, NH), 8.40 (1H, s, CHₐᵣₒₘ), 7.49-7.50 (1H, m, CHₐᵣₒₘ), 7.28-7.32 (2H, m, CHₐᵣₒₘ), 7.18-7.21 (1H, m, CHₐᵣₒₘ), 7.03-7.08 (2H, m, CHₐᵣₒₘ), 6.86-6.89 (1H, d, CHₐᵣₒₘ), 2.20 (3H, s, CH₃).

To a stirred solution of 0.25 g (0.67 mmol) of intermediate 12 in 19 mL of N,N-dimethylformamide is added 0.46 g (3.37 mmol) of potassium carbonate then 0.15 g (0.67 mmol) of 1-bromo-2-(2-bromoethoxy)ethane in 8 mL of NM-dimethylformamide for one hour. The reaction mixture is stirred at 75° C. for 20 hours. After returning to room temperature, the solvent is evaporated, water is added and the solid formed is filtered and dried under vacuum to afford 0.25 g (64%) of compound 29 as a beige powder.

LCMS (EI, m/z): (M+1) 443.30

¹H NMR: dH ppm (400 MHz, DMSO): 9.84 (1H, s, NH), 8.70 (1H, s, CHₐᵣₒₘ), 8.47 (1H, s, CHₐᵣₒₘ), 7.59 (1H, s, CHₐᵣₒₘ), 7.37-7.43 (2H, m, CHₐᵣₒₘ), 7.29-7.32 (1H, m, CHₐᵣₒₘ), 7.18-7.20 (1H, d, CHₐᵣₒₘ), 6.77-6.79 (1H, d, CHₐᵣₒₘ), 4.30 (2H, m, CH₂), 4.09-4.11 (2H, m, CH₂), 3.77-3.85 (4H, m, CH₂), 2.30 (3H, s, CH₃).

Compound 30:

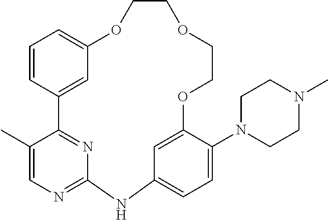

Compound 30 was prepared according to the protocol described for the preparation of compound 3 starting with 124 mg of compound 3 and 393 mg of the amine 1-methylpiperazine to afford 29 mg (22%) of compound 30.

LCMS (EI, m/z): (M+1) 462.55

¹H NMR: dH ppm (400 MHz, DMSO): 9.46 (1H, s, NH), 8.46-8.47 (1H, d, CHₐᵣₒₘ), 8.39 (1H, s, CHₐᵣₒₘ), 7.59 (1H, s, CHₐᵣₒₘ), 7.38-7.42 (1H, t, CHₐᵣₒₘ), 7.28-7.30 (1H, d, CHₐᵣₒₘ), 7.14-7.17 (1H, dd, CHₐᵣₒₘ), 6.73-6.80 (2H, m, CHₐᵣₒₘ), 4.29-4.31 (2H, m, CH₂), 4.07-4.10 (2H, m, CH₂), 3.80-3.82 (4H, m, CH₂), 2.89 (4H, m, CH₂), 2.42 (4H, m, CH₂), 2.29 (3H, m, CH₃), 2.19 (3H, m, CH₃).

Compound 31:

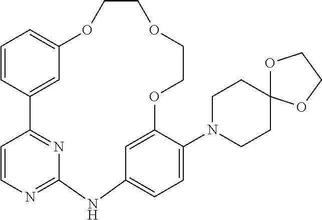 [SMILES: c1cc2cc(c1)-c1ccnc(n1)Nc1ccc(N3CCC4(CC3)OCCO4)c(c1)OCCOCCO2]

Compound 31 was prepared according to the protocol described for the preparation of compound 3 starting with 400 mg of compound 3 and 536 mg of the amine 1,4-dioxa-8-azaspiro[4.5]decane to afford 33 mg (7%) of compound 31.

LCMS (EI, m/z): (M+1) 491.55

¹H NMR: dH ppm (400 MHz, DMSO): 9.63 (1H, s, NH), 8.51-8.52 (1H, d, CHₐᵣₒₘ), 8.45-8.46 (1H, d, CHₐᵣₒₘ), 8.10 (1H, s, CHₐᵣₒₘ), 7.62-7.64 (1H, d, CHₐᵣₒₘ), 7.43-7.47 (1H, t, CHₐᵣₒₘ). 7.34-7.35 (1H, d, CHₐᵣₒₘ), 7.17-7.19 (1H, dd, CHₐᵣₒₘ). 6.79-6.88 (2H, m, CHₐᵣₒₘ), 4.22-4.27 (4H, m, CH₂), 3.90 (4H, s, CH₂), 3.79-3.85 (4H, m, CH₂), 2.98 (4H, m, CH₂), 1.73-1.76 (4H, m, CH₂).

Compound 32:

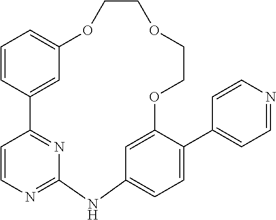 [SMILES: c1cc2cc(c1)-c1ccnc(n1)Nc1ccc(-c3ccncc3)c(c1)OCCOCCO2]

In a microwave reactor is mixed 95 mg (0.771 mmol) of pyridin-4-ylboronic acid dissolved in 10 mL of a 2:1 dioxane/H₂O mixture and are added successively 72 mg (0.17 mmol) of 2-dicyclohexylphosphino-2′,6′-dimethoxy-1,1′-biphenyl, 64 mg (0.070 mmol) of Pd₂(dba)₃, 297 mg (1.4 mmol) of K₃PO₄ and 300 mg (0.77 mmol) of compound 3. The reaction mixture is heated to 150° C. for 20 minutes. After returning to room temperature, the reaction is hydrolyzed by addition of water and extracted with ethyl acetate. The organic phase is dried over sodium sulfate, filtered and concentrated. The residue obtained is purified by chromatography on silica gel (eluent: ethyl acetate/methanol/ammonia: 94:4:2) to afford 96 mg (32%) of compound 32 as a yellow solid.

LCMS (EI, m/z): (M+1) 427.47

¹H NMR: dH ppm (400 MHz, DMSO): 10.04 (1H, s, NH), 8.71-8.22 (1H, d, CHₐᵣₒₘ), 8.60-8.62 (1H, d, CHₐᵣₒₘ), 8.50-8.54 (2H, d, CHₐᵣₒₘ), 8.14 (1H, m, CHₐᵣₒₘ), 7.64-7.66 (1H, d, CHₐᵣₒₘ), 7.55-7.57 (2H, d, CHₐᵣₒₘ), 7.45-7.49 (2H, m, CHₐᵣₒₘ), 7.37-7.39 (1H, d, CHₐᵣₒₘ), 7.18-7.21 (1H, dd, CHₐᵣₒₘ), 6.98-7.01 (1H, dd, CHₐᵣₒₘ), 4.22-4.26 m, CH₂), 3.78-3.84 (4H, m, CH₂).

Compound 33:

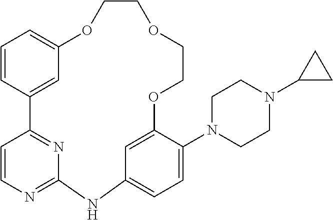 [SMILES: c1cc2cc(c1)-c1ccnc(n1)Nc1ccc(N3CCN(C4CC4)CC3)c(c1)OCCOCCO2]

Compound 33 was prepared according to the protocol described for the preparation of compound 3 starting with compound 3 and the amine 1-cyclopropylpiperazine.

LCMS (EI, m/z): (M+1) 474.57

¹H NMR: dH ppm (400 MHz, CDCl₃): 8.57-8.58 (1H, d, CHₐᵣₒₘ), 8.41-8.43 (1H, d, CHₐᵣₒₘ), 8.24 (1H, m, CHₐᵣₒₘ), 7.44-7.46 (1H, d, CHₐᵣₒₘ), 7.36-7.39 (1H, t, CHₐᵣₒₘ), 7.19 (1H, s, NH), 7.12-7.14 (1H, d, CHₐᵣₒₘ), 7.05-7.08 (1H, dd, CHₐᵣₒₘ), 6.85-6.88 (1H, m, CHₐᵣₒₘ), 6.51-6.55 (1H, d, CHₐᵣₒₘ), 4.40-4.43 (2H, t, CH₂), 4.30-4.32 (2H, t, CH₂), 3.97-4.00 (2H, t, CH₂), 3.90-3.92 (2H, t, CH₂), 3.04 (4H, m, CH₂), 2.81 (4H, m, CH₂), 1.61 (1H, m, CH), 0.47 (4H, m, CH₂).

Compound 34:

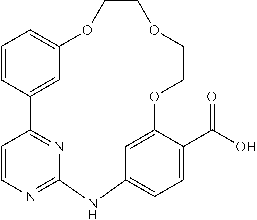 [SMILES: O=C(O)c1ccc2cc1OCCOCCOc1cccc(c1)-c1ccnc(n1)N2]

To 400 mg (0.93 mmol) of compound 3 in 10 mL of tetrahydrofuran is added 1.12 mL (2.8 mmol) of a solution of butyllithium in hexane (2.5 M) at −78° C. The reaction mixture is stirred at −78° C. for 30 minutes then CO₂ is bubbled through the solution for 3 hours. After returning to room temperature, the reaction is hydrolyzed by slow addition of water at 0° C. then a mixture of methanol/acetic acid is added. The solvents are evaporated, the solid formed is filtered and dried under vacuum to afford 367 mg (100%) of compound 34 as a beige powder.

LCMS (EI, m/z): (M+1) 394.39

Compound 35:

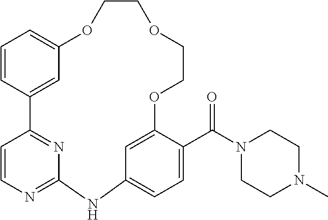 [SMILES: CN1CCN(C(=O)c2ccc3cc2OCCOCCOc2cccc(c2)-c2ccnc(n2)N3)CC1]

In a microwave reactor are added successively 367 mg (0.933 mmol) of compound 34, 815 μL of diisopropylethylamine (IDEA) (4.66 mmol), 333 μL (1.119 mmol) of T3P® and 112 mg (1.12 mmol) of 1-methylpiperazine. The reaction mixture is heated to 120° C. for 120 minutes. After returning to room temperature, the reaction is hydrolyzed by addition of water and extracted with dichloromethane. The organic phase is dried over sodium sulfate, filtered and concentrated. The residue obtained is purified by chromatography on silica gel (eluent: dichloromethane/methanol/ammonia: 94:4:2) to afford 38 mg (9%) of compound 35 as a yellow solid.

LCMS (EI, m/z): (M+1) 476.57

¹H NMR: dH ppm (400 MHz, CDCl₃): 8.71-8.72 (1H, d, CHₐᵣₒₘ), 8.47-8.48 (1H, d, CHₐᵣₒₘ), 8.22 (1H, m, CHₐᵣₒₘ), 7.45-7.47 (1H, d, CHₐᵣₒₘ), 7.37-7.41 (1H, t, CHₐᵣₒₘ), 7.33 (1H, s, NH), 7.18-7.21 (2H, m, CHₐᵣₒₘ), 7.06-7.09 (1H, dd, CHₐᵣₒₘ), 6.54-6.57 (1H, dd, CHₐᵣₒₘ), 4.25-4.38 (4H, m, CH₂), 3.89 (6H, m, CH₂), 3.39 (2H, m, CH₂), 2.35-2.52 (4H, t, CH₂), 2.33 (3H, s, CH₃).

Compound 36:

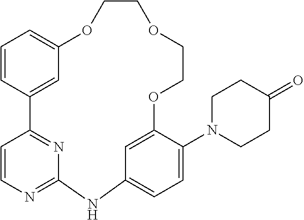 [SMILES: O=C1CCN(c2ccc3cc2OCCOCCOc2cccc(c2)-c2ccnc(n2)N3)CC1]

To 70 mg (0.14 mmol) of compound 31 is added dropwise 1 mL of a solution of hydrochloric acid in isopropanol (5N). The solution is stirred at 100° C. for 12 h. The solid formed is placed in basic medium and extracted with ethyl acetate. The organic phase is dried over sodium sulfate, filtered and concentrated to afford 38 mg (57%) of compound 36 as a yellow solid.

LCMS (EI, m/z): (M+1) 447.50

¹H NMR: dH ppm (400 MHz, DMSO) 9.67 (1H, s, NH), 8.52-8.54 (1H, d, CHₐᵣₒₘ), 8.51-8.52 (1H, d, CHₐᵣₒₘ), 8.11 (1H, s, CHₐᵣₒₘ), 7.63-7.65 (1H, d, CHₐᵣₒₘ), 7.43-7.47 (1H, t, CHₐᵣₒₘ), 7.35-7.37 (1H, d, CHₐᵣₒₘ), 7.17-7.20 (1H, dd, CHₐᵣₒₘ), 6.91-6.93 (1H, d, CHₐᵣₒₘ), 6.80-6.83 (1H, dd, CHₐᵣₒₘ), 4.24-4.29 (4H, m, CH₂), 3.86-3.89 (2H, t, CH₂), 3.80-3.82 (2H, m, CH₂), 3.23-3.26 (4H, t, CH₂), 2.45 (4H, m, CH₂).

Compound 37:

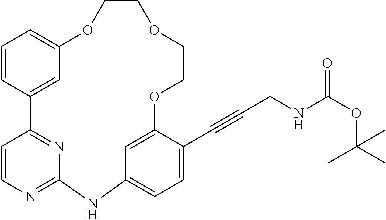 [SMILES: CC(C)(C)OC(=O)NCC#Cc1ccc2cc1OCCOCCOc1cccc(c1)-c1ccnc(n1)N2]

In a 50 mL round-bottom flask are mixed 1 g (2.335 mmol) of compound 3, 0.256 g (0.537 mmol) of 2-(dicyclohexylphosphino)-2′,4′,6′-triisopropylbiphenyl, 0.045 g (0.117 mmol) of benzonitrile palladium(II) chloride, 0.652 g (4.20 mmol) pro-2-yl-carbamic acid tent-butyl ester and 2.28 g (7 mmol) of cesium carbonate in 10 mL of dimethylformamide at room temperature. 0.90 g (2.80 mmol) of tetrabutylammonium bromide is added to the reaction mixture. The reaction mixture is heated to 80° C. for 15 hours. After returning to room temperature, the reaction is hydrolyzed by slow addition of water and extracted with ethyl acetate. The organic phase is dried over sodium sulfate, filtered and concentrated. The residue obtained is purified by chromatography on silica gel (eluent: ethyl acetate/cyclohexane: 40:60) to afford 144 mg (12%) of compound 37 as a yellow solid.

LCMS (EI, m/z): (M+1) 503.56

¹H NMR: dH ppm (400 MHz, DMSO): 10.02 (1H, s, NH). 8.58-8.62 (2H, m, CHₐᵣₒₘ), 809 (1H, s, CHₐᵣₒₘ), 7.64-7.66 (1H, d, CHₐᵣₒₘ), 7.44-7.48 (2H, m, CHₐᵣₒₘ), 7.33 (1H, m, CHₐᵣₒₘ), 7.17-7.24 (2H, m, CHₐᵣₒₘ), 6.83-6.86 (1H, m, CHₐᵣₒₘ), 4.25-4.27 (4H, m, CH₂), 3.96-3.97 (2H, m, CH₂), 3.80-3.86 (4H, m, CH₂), 1.40 (9H, s, CH₃).

Compound 38:

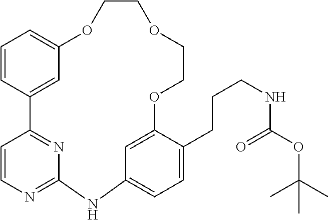 [SMILES: CC(C)(C)OC(=O)NCCCc1ccc2cc1OCCOCCOc1cccc(c1)-c1ccnc(n1)N2]

In a round-bottom flask and under argon, introduce 144 mg (0.28 mmol) of compound 37 in 10 mL of a mixture of THF/MeOH (1:1). Degas the mixture under argon and under vacuum. Add 30.5 mg (0.029 mmol) of Pd—C. Degas the mixture under argon and under vacuum then place in a round-bottom flask of hydrogen. The reaction mixture is stirred at 25° C. overnight then filtered on silica and rinsed with ethyl acetate, the solvent is evaporated and the solid formed is filtered and dried under vacuum to afford 132 mg (91%) of compound 38 as a yellow solid.

LCMS (EI, m/z): (M+1) 507.59

¹H NMR: dH ppm (400 MHz, DMSO): 9.70 (1H, s, NH), 8.54-8.55 (1H, d, CHₐᵣₒₘ), 8.45-8.47 (1H, d, CHₐᵣₒₘ), 8.12 (1H, s, CHₐᵣₒₘ), 7.62-7.64 (1H, d, CHₐᵣₒₘ), 7.43-7.47 (1H, t, CHₐᵣₒₘ), 7.36-7.38 (1H, d, CHₐᵣₒₘ), 7.17-7.18 (1H, dd, CHₐᵣₒₘ), 7.00-7.02 (1H, dd, CHₐᵣₒₘ), 6.76-6.80 (2H, m, CHₐᵣₒₘ), 4.17-4.25 (4H, m, CH₂), 3.79-3.89 (4H, m, CH₂), 2.89-2.93 (2H, m, CH₂), 2.40 (2H, m, CH₂), 1.56-1.67 (2H, m, CH₂), 1.38 (9H, s, CH₃).

Compound 39:

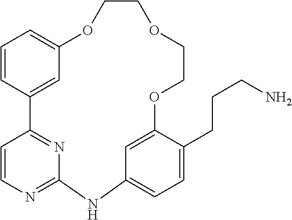 [SMILES: NCCCc1ccc2cc1OCCOCCOc1cccc(c1)-c1ccnc(n1)N2]

To 132 mg (0.261 mmol) of compound 38 is added dropwise 2 mL of a solution of hydrochloric acid in isopropanol (5N). The solution is stirred at 40° C. for 2 h 45 min. Evaporate to dryness. The solid formed is triturated in ether then recrystallized in isopropanol to obtain 100 mg (87%) of compound 39 as a yellow powder.

LCMS (EI, m/z): (M+1) 407.20

¹H NMR: dH ppm (400 MHz, DMSO): 9.78 (1H, s, NH), 8.56-8.57 (1H, d, CHₐᵣₒₘ), 8.50-8.51 (1H, d, CHₐᵣₒₘ), 8.12 (1H, m, CHₐᵣₒₘ), 7.64-7.66 (1H, d, CHₐᵣₒₘ), 7.45-7.49 (1H, t, CHₐᵣₒₘ), 7.39-7.41 (1H, d, CHₐᵣₒₘ), 7.18-7.21 (1H, dd, CHₐᵣₒₘ), 7.04-7.06 (1H, d, CHₐᵣₒₘ), 6.79-6.81 (1H, dd, CHₐᵣₒₘ), 4.21-4.25 (4H, m, CH₂), 3.88-3.91 (2H, t, CH₂), 3.80-3.82 (2H, m, CH₂), 2.73-2.79 (2H, m, CH₂), 2.54-2.58 (2H, t, CH₂), 1.76-1.84 (2H, m, CH₂).

Compound 40:

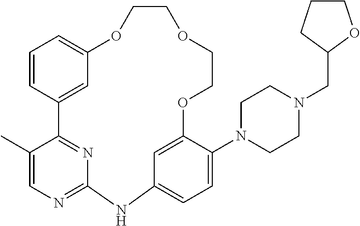 [SMILES: Cc1cnc2nc1-c1cccc(c1)OCCOCCOc1cc(ccc1N1CCN(CC3CCCO3)CC1)N2]

Compound 40 was prepared according to the protocol described for the preparation of compound 3 starting with 115 mg of compound 3 and 177 mg of the amine (1-((tetrahydrofuran-2-yl)methyl)piperazine) to afford 21 mg (15%) of compound 40.

LCMS (EI, m/z): (M+1) 532.64

¹H NMR: dH ppm (400 MHz, DMSO): 9.46 (1H, s, NH), 8.46-8.47 (1H, d, CHₐᵣₒₘ), 8.40 (1H, s, CHₐᵣₒₘ), 7.59 (1H, s, CHₐᵣₒₘ), 7.39-7.43 (1H, t, CHₐᵣₒₘ), 7.28-7.30 (1H, d, CHₐᵣₒₘ), 7.15-7.17 (1H, dd, CHₐᵣₒₘ), 6.74-6.81 (2H, m, CHₐᵣₒₘ), 4.29-4.31 (2H, m, CH₂), 4.07-4.12 (2H, m, CH₂), 3.90-3.97 (1H, m, CH), 3.78-3.82 (4H, m, CH₂), 3.70-3.76 (1H, m, CH), 3.57-3.62 (1H, m, CH), 2.89 (4H, m, CH₂), 2.49-2.68 (5H, m, CH₂), 2.38-2.41 (1H, m, CH), 2.29 (3H, m, CH₃), 1.88-1.96 (1H, m, CH), 1.73-1.83 (2H, m, CH₂), 1.43-1.52 (1H, m, CH).

Compound 41:

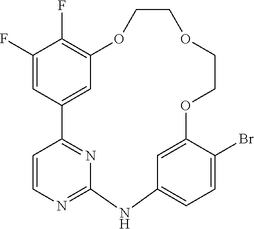 [SMILES: Fc1cc2cc(c1F)OCCOCCOc1cc(ccc1Br)Nc1nccc-2n1]

Step 1: Intermediate 13

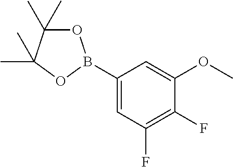

In a 50 mL round-bottom flask, introduce 1 g (4.48 mmol) of 5-bromo-2,3-difluoroanisole in 25 mL of dioxane then add 1.708 g (6.73 mmol) of bis(pinacolato)diboron, 1.320 g (13.45 mmol) of potassium acetate and 0.183 g (0.224 mmol) of bis(diphenylphosphino)ferrocene-palladium(II)dichloride dichloromethane complex. The reaction mixture is stirred at 80° C. overnight. After returning to room temperature, the reaction is hydrolyzed by slow addition of water and extracted with ethyl acetate. The organic phase is dried over sodium sulfate, filtered and concentrated. The residue obtained is purified by chromatography on silica gel (eluent: cyclohexane/ethyl acetate: 95:5) to afford 197 mg (15%) of intermediate 13 as a yellow solid.

LCMS (EI, m/z): (M+1) 271.08

¹H NMR: dH ppm (400 MHz, DMSO): 7.13-7.17 (2H, m, CHₐᵣₒₘ), 3.90 (3H, m, CH₃), 1.30 (12H, m, CH₃).

Step 2: Intermediate 14

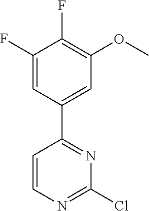 [SMILES: COc1cc(-c2ccnc(Cl)n2)cc(F)c1F]

In a microwave reactor are mixed 114 mg (0.771 mmol) of 2,4-dichloropyrimidine, 197 mg (0.729 mmol) of intermediate 13, 42.1 mg (0.036 mmol) of Pd(PPh₃)₄ and 6 mL of tetrahydrofuran. The reaction mixture is heated to 150° C. for 30 minutes. After returning to room temperature, the reaction is hydrolyzed by addition of water and extracted with ethyl acetate. The organic phase is dried over sodium sulfate, filtered and concentrated. The residue obtained is purified by chromatography on silica gel (eluent: cyclohexane/ethyl acetate: 70:30) to afford 112 mg (60%) of intermediate 14 as a white solid.

LCMS (EI, m/z): (M+1) 257.63

¹H NMR: dH ppm (400 MHz, DMSO): 8.88-8.90 (1H, d, CHₐᵣₒₘ), 8.27-8.28 (1H, d, CHₐᵣₒₘ), 7.85-7.90 (1H, m, CHₐᵣₒₘ), 7.79-7.81 (1H, m, CHₐᵣₒₘ), 4.02 (3H, m, CH₃).

Step 3: Intermediate 15

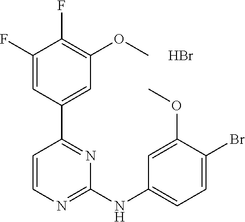 [SMILES: COc1cc(Nc2nccc(-c3cc(F)c(F)c(OC)c3)n2)ccc1Br ; Br]

To a solution of 1.25 g (4.87 mmol) of intermediate 14 and 0.984 g (4.87 mmol) of 4-bromo-3-methoxyaniline in 28 mL of n-butanol is added 0.97 mL of hydrochloric acid solution (5N). The reaction mixture is stirred at 80° C. overnight. After returning to room temperature, the solid formed is filtered then rinsed with n-butanol to afford 1.4 g (68%) of intermediate 15 as a yellow powder.

LCMS (EI, m/z): (M+1) 423.22

¹H NMR: dH ppm (400 MHz, DMSO): 9.90 (1H, s, NH), 8.92-8.94 (1H, d, CHₐᵣₒₘ), 7.82-7.87 (1H, m, CHₐᵣₒₘ), 7.78-7.80 (1H, d, CHₐᵣₒₘ), 7.74-7.75 (1H, d, CHₐᵣₒₘ), 7.57-7.58 (1H, d, CHₐᵣₒₘ), 7.47-48 (1H, d, CHₐᵣₒₘ), 7.38-7.41 (1H, dd, CHₐᵣₒₘ), 4.01 (3H, s, CH₃), 3.86 (3H, s, CH₃).

Step 4: Intermediate 16

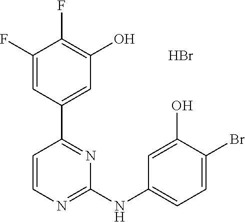 [SMILES: Oc1cc(Nc2nccc(-c3cc(O)c(F)c(F)c3)n2)ccc1Br ; Br]

To a solution of 1.4 g (3.32 mmol) of intermediate 15 in 36 mL of dichloromethane is added 1.56 mL (16.58 mmol) of tribromo-borane at −78° C. The reaction mixture is then stirred at 45° C. for 4 h then overnight at room temperature. 20 mL of methanol is added at 0° C. to the reaction mixture which is then heated to 35° C. for 25 minutes. The solid formed is filtered then washed twice with 20 mL of ether to afford 1.54 g (98%) of intermediate 16 as a yellow powder.

LC-MS (EI m/z): (M+H⁺): 395.17+397.2

¹H NMR: dH ppm (400 MHz, DMSO): 9.75 (1H, s, NH), 8.55-8.56 (1H, d, CHₐᵣₒₘ), 7.61-7.63 (2H, m, CHₐᵣₒₘ), 7.49-7.50 (1H, d, CHₐᵣₒₘ), 7.35-7.37 (2H, m, CHₐᵣₒₘ), 7.23-7.26 (1H, dd, CHₐᵣₒₘ).

Step 5: Compound 41

To a stirred solution of 0.7 g (1.47 mmol) of intermediate 16 in 70 mL of N,N-dimethylformamide is added 0.34 g (1.47 mmol) of potassium carbonate then 0.32 g (1.47 mmol) of 1-bromo-2-(2-bromoethoxy)ethane in 35 mL of N,N-dimethylformamide for one hour. The reaction mixture is stirred at 75° C. for 20 hours. After returning to room temperature, the solvent is evaporated, water is added and the solid formed is filtered and dried under vacuum to afford 0.53 g (78%) of compound 41 as a beige powder.

LCMS (EI, m/z): (M+1) 465.26

¹H NMR: dH ppm (400 MHz, DMSO): 9.98 (1H, s, NH), 8.60-8.60 (1H, d, CHₐᵣₒₘ), 8.54 (1H, s, CHₐᵣₒₘ), 8.05-8.07 (1H, d, CHₐᵣₒₘ), 7.81-7.85 (1H, m, CHₐᵣₒₘ), 7.43-7.47 (2H, m, CHₐᵣₒₘ), 6.83-6.85 (1H, dd, CHₐᵣₒₘ), 4.41 (2H, m, CH₂), 4.23 (2H, m, CH₂), 3.81 (4H, m, CH₂).

Compound 42:

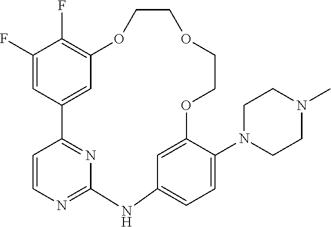 [SMILES: CN1CCN(c2ccc3cc2OCCOCCOc2cc(cc(F)c2F)-c2ccnc(n2)N3)CC1]

Compound 42 was prepared according to the protocol described for the preparation of compound 3 starting with 272 mg of compound 41 and 117 mg of the amine 1-methylpiperazine to afford 14 mg of compound 42 with a yield of 8%.

LCMS (EI, m/z): (M+1) 484.51

¹H NMR: dH ppm (400 MHz, DMSO): 9.66 (1H, s, NH), 8.53-8.55 (1H, d, CHₐᵣₒₘ), 8.34-8.35 (1H, d, CHₐᵣₒₘ), 8.07-8.09 (1H, d, CHₐᵣₒₘ), 7.82-7.87 (1H, m, CHₐᵣₒₘ), 7.39-7.40 (1H, d, CHₐᵣₒₘ), 6.82-6.87 (2H, m, CHₐᵣₒₘ), 4.37-4.41 (2H, m, CH₂), 4.21-4.23 (2H, m, CH₂), 3.75-3.81 (4H, m, CH₂), 2.95 (4H, m, CH₂), 2.46 (4H, m, CH₂), 2.21 (3H, m, CH₃).

Compound 43:

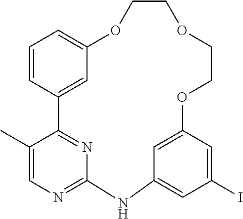 [SMILES: Cc1cnc2nc1-c1cccc(c1)OCCOCCOc1cc(I)cc(c1)N2]

Step 1: Intermediate 17

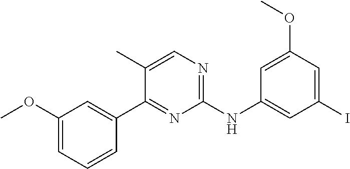 [SMILES: COc1cc(I)cc(Nc2ncc(C)c(-c3cccc(OC)c3)n2)c1]

To a solution of 0.44 g (1.79 mmol) of intermediate 10 and 0.446 g (1.790 mmol) of 3-iodo-5-methoxyaniline in 10 mL of n-Butanol is added 0.35 mL of hydrochloric acid solution (5N). The reaction mixture is stirred at 80° C. overnight. After returning to room temperature, the reaction is hydrolyzed by slow addition of NaHCO₃ solution and extracted with ethyl acetate. The organic phase is dried over sodium sulfate, filtered and concentrated. The residue obtained is purified by chromatography on silica gel (eluent: cyclohexane/ethyl acetate: 90:10) to afford 441 mg (55%) of intermediate 17 as a white solid.

LCMS (EI, m/z): (M+1) 448.27

Step 2: Intermediate 18

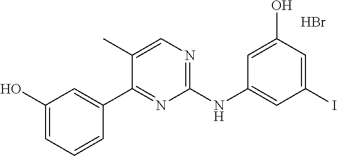 [SMILES: Cc1cnc(Nc2cc(O)cc(I)c2)nc1-c1cccc(O)c1 ; Br]

To a solution of 441 mg (0.98 mmol) of intermediate 17 in 11 mL of dichloromethane is added 0.47 mL (4.94 mmol) of tribromo-borane at −78° C. The reaction mixture is then stirred at 45° C. for 5 hours then overnight at room temperature. 20 mL of methanol is added at 0° C. to the reaction mixture which is heated to 35° C. for 25 minutes. The solid formed is filtered then washed twice with 20 mL of ether to afford 0.50 g (99%) of intermediate 18 as a yellow powder.

LCMS (EI, m/z): (M+1) 420.01

¹H NMR: dH ppm (400 MHz, DMSO): 9.58 (1H, s, NH), 8.42 (1H, s, CHₐᵣₒₘ), 7.70 (1H, s, CHₐᵣₒₘ), 7.29-7.33 (2H, m, CHₐᵣₒₘ), 7.04-7.09 (2H, m, CHₐᵣₒₘ), 6.87-6.90 (1H, d, CHₐᵣₒₘ), 6.69 (1H, d, Hₐᵣₒₘ), 2.21 (3H, s, CH₃).

Step 3: Compound 43

To a stirred solution of 0.54 g (1.09 mmol) of intermediate 18 in 55 mL of N,N-dimethylformamide is added 0.75 g (5.47 mmol) of potassium carbonate then 0.137 mL (1.09 mmol) of 1-bromo-2-(2-bromoethoxy)ethane in 23 mL of N,N-dimethylformamide for one hour. The reaction mixture is stirred at 80° C. for 20 hours. After returning to room temperature, the solvent is evaporated, water is added and the solid formed is filtered and dried under vacuum to afford 0.25 g (64%) of compound 43 as a beige powder.

LCMS (EI, m/z): (M+1) 490.30

¹H NMR: dH ppm (400 MHz, DMSO): 9.74 (1H, s, NH), 8.62 (1H, s, CHₐᵣₒₘ), 8.47 (1H, s, CHₐᵣₒₘ), 7.74 (1H, s, CHₐᵣₒₘ), 7.41-7.42 (2H, m, CHₐᵣₒₘ), 7.20 (1H, s, CHₐᵣₒₘ), 7.09-7.14 (1H, m, CHₐᵣₒₘ), 6.82 (1H, s, CHₐᵣₒₘ), 4.30-4.33 (2H, t, CH₂), 4.00-4.02 (2H, t, CH₂), 3.82-3.86 (4H, m, CH₂), 2.38 (3H, s, CH₃).

Compound 44:

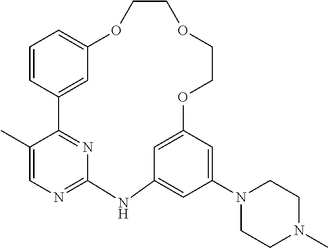

Compound 44 was prepared according to the protocol described for the preparation of compound 3 starting with 490 mg of compound 43 and 401 mg of the amine 1-methylpiperazine to afford 156 mg (34%) of compound 44.

LCMS m/z): (M+1) 462.5

¹H NMR: dH ppm (400 MHz, DMSO): 9.36 (1H, s, NH), 8.42 (1H, s, CHₐᵣₒₘ), 8.07 (1H, s, CHₐᵣₒₘ), 7.76 (1H, s, CHₐᵣₒₘ), 7.40-7.41 (1H, d, CHₐᵣₒₘ), 7.08-7.12 (1H, m, CHₐᵣₒₘ), 6.41 (1H, s, CHₐᵣₒₘ), 6.10 (1H, s, CHₐᵣₒₘ), 4.30-4.32 (2H, t, CH₂), 3.99-4.00 (2H, t, CH₂), 3.81-3.86 (4H, m, CH₂), 3.79 (4H, m, CH₂), 2.44 (4H, m, CH₂), 2.36 (3H, m, CH₃), 2.22 (3H, m, CH₃).

Compound 45:

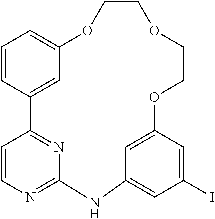 [SMILES: Ic1cc2cc(c1)OCCOCCOc1cccc(c1)-c1ccnc(n1)N2]

Step 1: 2-chloro-4-(3-methoxyphenyl)pyrimidine (Intermediate 19)

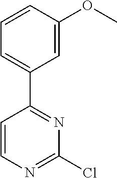

To 11.32 g (76 mmol) of 2,4-dichloropyrimidine in 550 mL of anhydrous THF is added 11 g (72.4 mmol) of 3-methoxyphenyl-boronic acid. The reaction mixture is stirred at room temperature for 10 minutes then 19.1 g of sodium carbonate dissolved in 80 mL of water then 4.18 g (3.62 mmol) of tetrakis(triphenylphosphine)palladium(0) is added at room temperature. The reaction mixture is stirred at 90° C. for 16 hours. Ethyl acetate is added and the organic phase is washed with saturated sodium chloride solution, dried over magnesium sulfate, filtered. The filtrate is evaporated and the residue purified by silica column chromatography using Companion® (eluent: heptane/ethyl acetate: 0 to 10%, then dichloromethane/ethyl acetate: 10%) to afford 15.23 g (95%) of 2-chloro-4-(3-methoxyphenyl)pyrimidine as a white solid.

LCMS (EI, m/z): (M+1) 221.65

¹H NMR: dH ppm (400 MHz, DMSO): 8.82-8.84 (1H, d, CHₐᵣₒₘ), 8.17-8.19 (1H, d, 7.77-7.79 (1H, d, CHₐᵣₒₘ), 7.70-7.71 (1H, d, CHₐᵣₒₘ), 7.47-7.51 (1H, d, CHₐᵣₒₘ), 7.17-7.20 (1H, t, CHₐᵣₒₘ), 3.86 (3H, s, CH₃).

Step 2: Intermediate 20

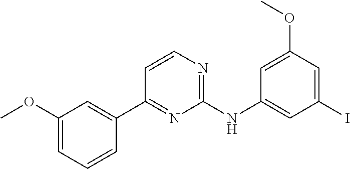 [SMILES: COc1cc(I)cc(Nc2nccc(-c3cccc(OC)c3)n2)c1]

To a solution of 0.5 g (2.26 mmol) of intermediate 19 and 0.56 g (2.26 mmol) of 3-iodo-5-methoxyaniline in 13 mL of n-butanol is added 0.45 mL of hydrochloric acid solution (5N). The reaction mixture is stirred at 80° C. overnight. After returning to room temperature, the reaction is hydrolyzed by slow addition of NaHCO₃ solution and extracted with ethyl acetate. The organic phase is dried over sodium sulfate, filtered and concentrated. The residue obtained is purified by chromatography on silica gel (eluent: cyclohexane/ethyl acetate: 80:20) to afford 693 mg (70%) of intermediate 20 as a yellow solid.

LCMS (EI, m/z): (M+1) 434.24

Step 3: Intermediate 21

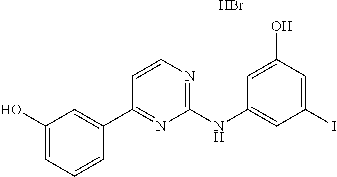 [SMILES: Br ; Oc1cc(I)cc(Nc2nccc(-c3cccc(O)c3)n2)c1]

To a solution of 693 mg (1.6 mmol) of intermediate 20 in 18 mL of dichloromethane is added 0.75 mL (1.6 mmol) of tribromo-borane at −78° C. The reaction mixture is then stirred at 45° C. for 5 h then overnight at room temperature. 20 mL of methanol is added at 0° C. to the reaction mixture which is then heated to 35° C. for 25 minutes. The solid formed is filtered then washed twice with 20 mL of ether to afford 0.76 g (98%) of intermediate 21 as a yellow powder.

LCMS (EI, m/z): (M+1) 406.,18

¹H NMR: dH ppm (400 MHz, DMSO): 9.69 (1H, s, NH), 8.54-8.55 (1H, d, CHₐᵣₒₘ), 7.79 (1H, s, CHₐᵣₒₘ), 7.58-7.60 (1H, d, CHₐᵣₒₘ), 7.52 (1H, m, CHₐᵣₒₘ), 7.32-7.36 (3H, m, CHₐᵣₒₘ), 6.94-6.96 (1H, dd, CHₐᵣₒₘ), 6.75 (1H, t, CH₃).

Step 4: Compound 45

To a stirred solution of 0.76 g (1.56 mmol) of intermediate 21 in 78 mL of N,N-dimethylformamide is added 1.08 g (7.82 mmol) of potassium carbonate then 0.19 mL (1.56 mmol) of 1-bromo-2-(2-bromoethoxy)ethane in 34 mL of N,N-dimethylformamide for one hour. The reaction mixture is stirred at 80° C. for 20 hours. After returning to room temperature, the solvent is evaporated, water is added and the solid formed is filtered and dried under vacuum to afford 0.30 g (40%) of compound 45 as a beige powder,

LCMS (EI, m/z) 476.28

Compound 46:

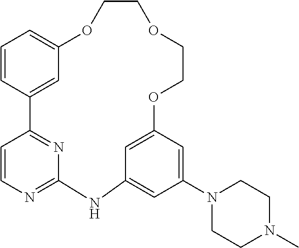 [SMILES: CN1CCN(c2cc3cc(c2)OCCOCCOc2cccc(c2)-c2ccnc(n2)N3)CC1]

Compound 46 was prepared according to the protocol described for the preparation of compound 3 starting with 300 mg of compound 45 and 253 mg of the amine 1-methylpiperazine to afford 115 mg of compound 46 with a yield of 41%,

LCMS (EI, m/z): (M+1) 448.52

¹H NMR: dH ppm (400 MHz, DMSO): 9.54 (1H, s, NH), 8.54-8.55 (1H, d, CHₐᵣₒₘ), 8.27 (1H, S, CHₐᵣₒₘ), 8.14 (1H, s, CHₐᵣₒₘ), 7.70-72 (1H, d, CHₐᵣₒₘ), 7.44-7.46 (1H, d, CHₐᵣₒₘ), 7.40-7.42 (1H, d, CHₐᵣₒₘ), 7.08-7.11 (1H, dd, CHₐᵣₒₘ), 6.44 (1H, s, CHₐᵣₒₘ), 6.16 (1H, s, CHₐᵣₒₘ), 4.28-4.31 (2H, t, CH₂), 4.02-4.05 (2H, t, CH₂), 3.87-3.93 (4H, m, CH₂), 3.16 (4H, m, CH₂), 2.45 (4H, m, CH₂), 2.23 (3H, m, CH₃)

2. Biological Activity of the Compounds According to the Invention

The following abbreviations were used:

ATP: Adenosine-5′-triphosphate

IMDM: Iscove's Modified Dulbecco's Medium

PSFG: Penicillin Streptomycin FunGizone

RPMI: Roswell Park Memorial Institute medium

FCS: Fetal calf serum

Measurement of the In Vitro Inhibitory Activities of the Compounds According to the Invention:

FLT3 (#PV3182), JAK2 (#FV4210) and JAK3 (#PV3855) recombinant enzymes were purchased from Life Technologies, FLT3-ITD (#0778-0000-1) and FLT3^{D835Y} (#14-610) proteins were purchased from ProQinase and Merck Millipore, respectively. All the tests were carried out in 384-well plates. The principle of these binding tests is based on the LanthaScreen® TR-FRET methodology from Life Technologies.

FLT3 tests. The reaction mixture (15 μL total volume) contains the following compounds: 15 nM FLT3, FLT3-ITD or FLT3^{D835Y}, 3 nM kinase tracer 236 (Life Technologies, #PV5592) and 6 nM LanthaScreen® anti-GST antibody coupled to a europium chelate (Life Technologies, #PV5594) for FLT3-ITD and FLT3^{D835Y} or 6 nM LanthaScreen® anti-His antibody coupled to a europium chelate (Life Technologies, #PV5596) for FLT3.

JAK tests. The reaction mixture (15 μL total volume) contains the following compounds: 15 nM JAK2 or JAK3, 150 nM kinase tracer 236 (Life Technologies, #PV5592) for JAK2 or 3 nM for JAK3 and 6 nM LanthaScreen® anti-GST antibody coupled to a europium chelate (Life Technologies, #PV5594) for both enzymes.

The compounds are evaluated at 8 different concentrations prepared by making dilutions from a starting 10 mM stock solution in dimethylsulfoxide (DMSO) (Sigma, #D8418). The final DMSO concentration in the test is 1%. The reaction is carried out at 25° C. for 1 hour and detected on the EnVision® reader (Perkin Elmer) according to the recommendations of the supplier, Life Technologies.

The results are presented (Table 1) as the concentration values of the compound at which 50% of the kinase activity is inhibited, IC₅₀ (μM), generated using the PRISM software (GraphPad).

TABLE 1

| | | | | | JAK3 |
|---|---|---|---|---|---|
| Compound/ | FLT3 | FLT3-ITD | FLT3^{D835Y} | JAK2 | IC₅₀ |
| enzyme | IC₅₀ (μm) | IC₅₀ (μm) | IC₅₀ (μm) | IC₅₀ (μm) | (μm) |
| 4 | 0.0255 | 0.0145 | 0.035 | 0.0077 | 0.05 |
| 46 | 0.0047 | 0.0016 | 0.0062 | 0.060 | 0.10 |
| 6 | 0.11 | 0.12 | 0.0412 | 0.0052 | 0.05 |

In Vitro Measurement of the Antiproliferative Activity of the Compounds According to the Invention:

Cell Lines.

The characteristics of the cell lines used are as follows (Table 2).

TABLE 2

| | | | | Basal | Seeding |
|---|---|---|---|---|---|
| | | Tumor | Oncogene | culture | (Cell |
| Cell lines | Supplier | origin | expressed | medium | density/well) |
| MV4-11 | DSMZ | Acute | FLT3-ITD | IMDM, | 0.4 · 10⁵ |
| | | myeloid | | 10% FCS, | cells/well |
| | | leukemia | | PSFG | (100 μL) |
| MOLM-13 | DSMZ | Acute | FLT3-ITD | RPMI | 0.3 · 10⁵ |
| | | myeloid | | 1640, 15% | cells/well |
| | | leukemia | | FCS, | (100 μL) |
| | | | | PSFG | |

Measurement of Antiproliferative Activity.

MV4-11 and MOLM-13 cell lines are cultured in the culture medium specified in Table 2 above and according to the supplier's recommendations. The tests are carried out in 96-well plates. The cells are divided in two at D0. At D1, they are seeded and treated with the compounds at various concentrations and incubated for 72 h at 37° C. and 5% CO₂. The dilution of the compounds from stock solutions in DMSO (Sigma, #D8418) was made semi-logarithmically for a final concentration in the culture medium of 0.1%. At day 4, cell viability is evaluated by assaying the ATP released by the living cells using the ATPLite® kit (Perkin Elmer, #6016947). The EC₅₀ values (concentration of the compound necessary to obtain 50% of the maximum effect) are calculated using curve-fitting software. The results in the form of EC₅₀ values (in M) are presented in Table 3.

TABLE 3

| Compound/line | MV411 | MOLM13 |
|---|---|---|
| 4 | 5.73E−08 | 1.89E−08 |
| 46 | 1.80E−09 | 3.90E−09 |
| 6 | 2.46E−07 | 1.45E−07 |

1-17. (canceled)

18. A compound of the following general formula (I): 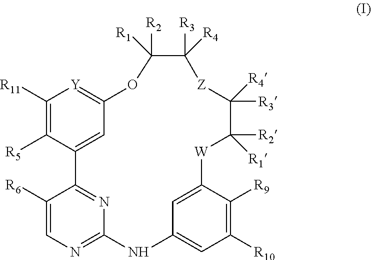
or a pharmaceutically acceptable salt and/or solvate thereof,
wherein:
W represents an oxygen or sulfur atom,
Y represents a nitrogen atom or a CRy group wherein Ry represents a hydrogen atom, a halogen atom, a (C₁-C₆)alkyl, (C₁-C₆)haloalkyl, (C₁-C₆)alkoxy, (C₁-C₆)haloalkoxy, OH, CN, NO₂, NR₁₂R₁₃, CO₂H or CO₂((C₁-C₆)alkyl) group,
Z represents a (CR_{Q1}R_{Q2})ₙQ(CR_{Q3}R_{Q4})ₘ group, wherein n and m represent, independently of each other, an integer between 0 and 3,
Q represents O, S, S(O) or S(O)₂,
R_{Q1}, R_{Q2}, R_{Q3} and R_{Q4} represent, independently of each other, a hydrogen atom or a (C₁-C₆)alkyl group,
R₁, R₂, R₁′ and R₂′ represent, independently of each other, a hydrogen atom or a (C₁-C₆)alkyl group,
R₃, R₄, R₃′ and R₄′ represent, independently of each other, a hydrogen atom, a (C₁-C₆)alkyl or OH group or R₃ and R₄ and/or R₃′ and R₄′ together form, with the carbon atom that bears them, an optionally substituted monocyclic carbocycle or heterocycle,
R₅ and R₆ represent, independently of each other, a hydrogen atom, a halogen atom, a (C₁-C₆)alkyl, (C₁-C₆)haloalkyl, (C₁-C₆)alkoxy, (C₁-C₆)haloalkoxy, (C₁-C₆)thioalkoxy, (C ₁-C₆)halothioalkoxy, OH, SH, CN, NO₂, or NR₇R₈ group,
R₉ and R₁₀ represent, independently of each other, a hydrogen atom, a halogen atom, an optionally substituted (C₁-C₆)alkyl, optionally substituted (C₂-C₆)alkenyl, optionally substituted (C₂-C₆)alkynyl, optionally substituted (C₁-C₆)alkoxy, optionally substituted (C₁-C₆)thioalkoxy, CN, NO₂, OH, SH, NR₁₄R₁₅, CO₂R₅₄, CONR₅₅R₅₆ group, an optionally substituted carbocycle or an optionally substituted heterocycle,
R₁₁ represents a hydrogen atom, a halogen atom, or a (C₁-C₆)alkyl, (C₁-C₆)haloalkyl, (C₁-C₆)alkoxy or (C₁-C₆)haloalkoxy group, and
R₇, R₈, R₁₂, R₁₃, R₁₄, R₁₅, R₅₄, R₅₅ and R₅₆ represent, independently of each other, a hydrogen atom or an optionally substituted (C₁-C₆)alkyl, optionally substituted (C₂-C₆)alkenyl, or optionally substituted (C₂-C₆)alkynyl group, or R₇ and R₈, R₁₂ and R₁₃, R₁₄ and R₁₅ and/or R₅₅ and R₅₆, independently of each other, form with the nitrogen atom that bears them an optionally substituted nitrogen containing heterocycle.

19. The compound according to claim 18, wherein Z represents an oxygen atom and R₁, R₂, R₃, R₄, R₁′, R₂′, R₃′ and R₄′ each represent a hydrogen atom.

20. The compound according to claim 18, wherein Y represents a CRy group wherein Ry represents a hydrogen atom or a halogen atom.

21. The compound according to claim 18, wherein R₁₁ represents a hydrogen atom or a halogen atom.

22. The compound according to claim 18, wherein R₅ and R₆ represent, independently of each other, a hydrogen atom or a (C₁-C₆)alkyl group.

23. The compound according to claim 18, wherein R₉ and R₁₀ represent, independently of each other, a hydrogen atom, a halogen atom, CO₂R₅₄, CONR₅₅R₅₆, or a (C₁-C₆)alkyl, (C₂-C₆)alkenyl, (C₂-C₆)alkynyl, (C₁-C₆)alkoxy, (C₁-C₆)thioalkoxy, (C₁-C₆)alkyl-amino, di((C₁-C₆)alkyl)amino or heterocycle group, said group being optionally substituted by one or more substituents selected from:
a halogen atom,
a (C₁-C₆)alkyl group optionally substituted by one or more groups selected from a halogen atom, OR₁₆, SR₁₇, NR₁₈R₁₉, a carbocycle and a heterocycle,
oxo (═O), CN, NO₂, OR₂₀, SR₂₁, NR₂₂R₂₃, C(O)R₂₄, CO₂R₂₅, OC(O)R₂₆, S(O)R²⁷, SO₂R₂₈, NR₂₉C(O)R₃₀, C(O)NR₃₁R₃₂, NR₃₃CO₂R₃₄, OC(O)NR₃₅R₃₆, NR₃₇CONR₃₈R₃₉ and OCO₂R₄₀ groups,
a carbocycle optionally substituted by one or more groups selected from a halogen atom, a (C₁-C₆)alkyl group, oxo (═O), OR₄₁, SR₄₂ and NR₄₃R₄₄,
a heterocycle optionally substituted by one or more groups selected from a halogen atom, a (C₁-C₆)alkyl group, oxo (═O), OR₄₅, SR₄₆ and NR₄₇R₄₈, and
an —O(CH₂)ₙO— group wherein n represents an integer between 1 and 5,
wherein:
R₁₆ to R₄₈ represent, independently of each other, a hydrogen atom, a (C₁-C₆)alkyl, aryl, aryl-(C₁-C₆)alkyl, heterocycle or heterocycle-(C₁-C₆)alkyl group,
the aryl ring of these groups being optionally substituted by one or more groups selected from a halogen atom and a (C₁-C₆)alkyl group, and
the heterocyclic ring of these groups being optionally substituted by one or more groups selected from a halogen atom, a (C₁-C₆)alkyl group, and oxo (═O), or
R₂₂ and R₂₃, R₃₁ and R₃₂, R₃₅ and R₃₆, R₃₈ and R₃₉, R₄₃ and R₄₄, and/or R₄₇ and R₄₈ together form, with the nitrogen atom that bears them, a nitrogen containing heterocycle optionally substituted by one or more groups selected from a halogen atom, a (C₁-C₆)alkyl group, and oxo (═O).

24. The compound according to claim 18, wherein R₉ and R₁₀ represent, independently of each other:
a hydrogen or halogen atom,
a CO₂R₅₄ group wherein R₅₄ represents a hydrogen atom or a (C₁-C₆)alkyl group,
a CONR₅₅R₅₆ group wherein R₅₅ and R₅₆ form with the nitrogen atom that bears them a nitrogen containing heterocycle having 5, 6 or 7 members, comprising 1 or 2 heteroatoms selected from N and O of which at least one is a nitrogen atom, optionally substituted by one or more substituents selected from a halogen atom, (C₁-C₆)alkyl, oxo (═O), OR₂₀, NR₂₂R₂₃, CO₂R₂₅, C(O)NR₃₁R₃₂, NR₃₃CO₂R₃₄, OC(O)NR₃₅R₃₆, NR₃₇CONR₃₈R₃₉ and OCO₂R₄₀,
a —Z₁—(CH₂)ₘ—R₄₉ group wherein Z₁ represents a single bond, CH₂—CH₂, CH═CH, C≡C, O, S or NR₅₀; m represents an integer between 1 and 6; R₅₀ represents a hydrogen atom or a (C₁-C₆)alkyl group; and R₄₉ represents a halogen atom, OR₂₀, NR₂₂R₂₃, C(O)R₂₄, CO₂R₂₅, OC(O)R₂₆, NR₂₉C(O)R₃₀, C(O)NR₃₁R₃₂, NR₃₃CO₂R₃₄, OC(O)NR₃₅R₃₆, NR₃₇CONR₃₈R₃₉ or OCO₂R₄₀, or
a heterocycle having 5, 6 or 7 members, comprising 1 or 2 heteroatoms selected from N and O, optionally substituted by one or more substituents selected from:
a halogen atom,
a (C₁-C₆)alkyl group optionally substituted by one or more groups selected from a halogen atom, OR₁₆, NR₁₈R₁₉, a C₃ to C₆ monocyclic carbocycle and a 3- to 6-membered monocyclic heterocycle comprising 1 or 2 heteroatoms selected from N and O,
oxo (═O), OR₂₀, NR₂₂R₂₃, C(O)R₂₄, CO₂R₂₅, OC(O)R₂₆, NR₂₉C(O)R₃₀, C(O)NR₃₁R₃₂, NR₃₃CO₂R₃₄, OC(O)NR₃₅R₃₆, NR₃₇CONR₃₈R₃₉ or OCO₂R₄₀ groups,
a C₃ to C₆ carbocycle optionally substituted by one or more groups selected from a halogen atom, a (C₁-C₆)alkyl group, oxo (═O), OR₄₁ and NR₄₃R₄₄,
a 3- to 6-membered heterocycle comprising 1 or 2 heteroatoms selected from N and O, optionally substituted by one or more groups selected from a halogen atom, a (C₁-C₆)alkyl group, oxo (═O), OR₄₅ and NR₄₇R₄₈, and
an —O(CH₂)ₙO— group wherein n represents an integer equal to 2 or 3,
wherein:
R₁₆, R₁₈ to R₂₀, R₂₂ to R₂₆, R₂₉ to R₄₀, R₄₅ and R₄₇ to R₄₈ represent, independently of each other, a hydrogen atom, a (C₁-C₆)alkyl, aryl, or aryl-(C₁-C₆)alkyl group,
the aryl ring of these groups being a phenyl group and being optionally substituted by one or more groups selected from a halogen atom and a (C₁-C₆)alkyl group, or
R₂₂ and R₂₃, R₃₁ and R₃₂, R₃₅ and R₃₆, R₃₈ and R₃₉, R₄₃ and R₄₄, and/or R₄₇ and R₄₈ together form, with the nitrogen atom that bears them, a 5- or 6-membered nitrogen containing heterocycle, optionally comprising 1 heteroatom in addition to the nitrogen atom selected from N and O, the heterocycle being optionally substituted by one or more groups selected from a halogen atom, a (C₁-C₆)alkyl group, and oxo (═O).

25. The compound according to claim 18, wherein it is selected from the following compounds: 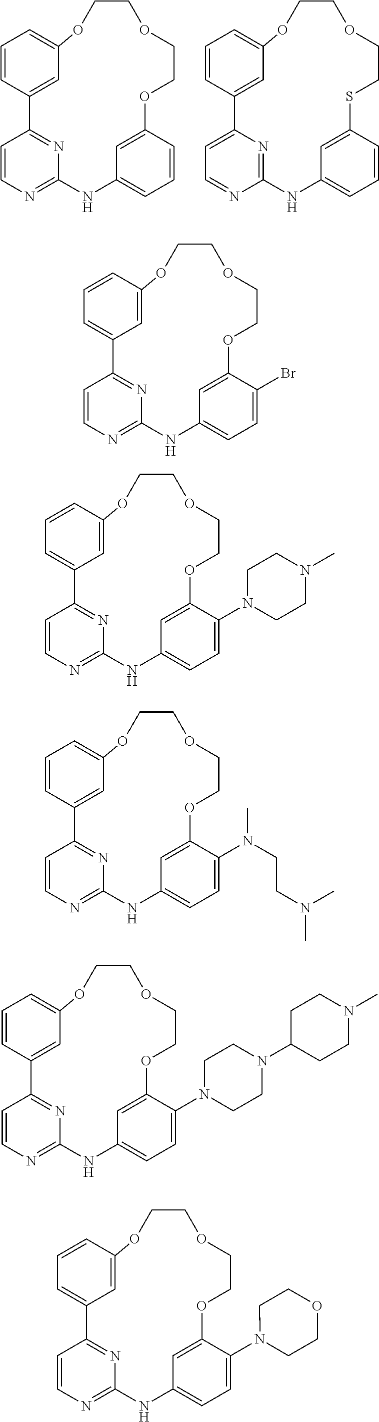 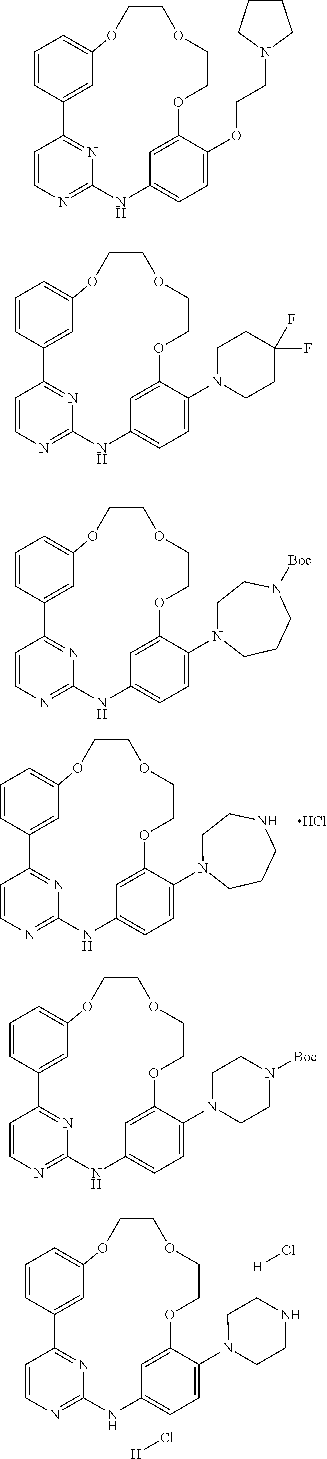 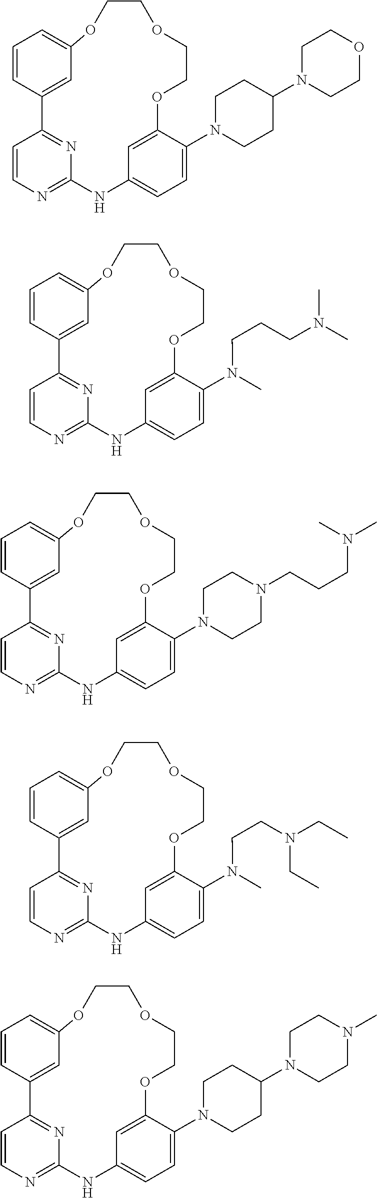 [SMILES: c1cc2cc(c1)-c1ccnc(n1)Nc1ccc(N3CCC(N4CCOCC4)CC3)c(c1)OCCOCCO2 ; CN(C)CCCN(C)c1ccc2cc1OCCOCCOc1cccc(c1)-c1ccnc(n1)N2 ; CN(C)CCCN1CCN(c2ccc3cc2OCCOCCOc2cccc(c2)-c2ccnc(n2)N3)CC1 ; CCN(CC)CCN(C)c1ccc2cc1OCCOCCOc1cccc(c1)-c1ccnc(n1)N2 ; CN1CCN(C2CCN(c3ccc4cc3OCCOCCOc3cccc(c3)-c3ccnc(n3)N4)CC2)CC1] 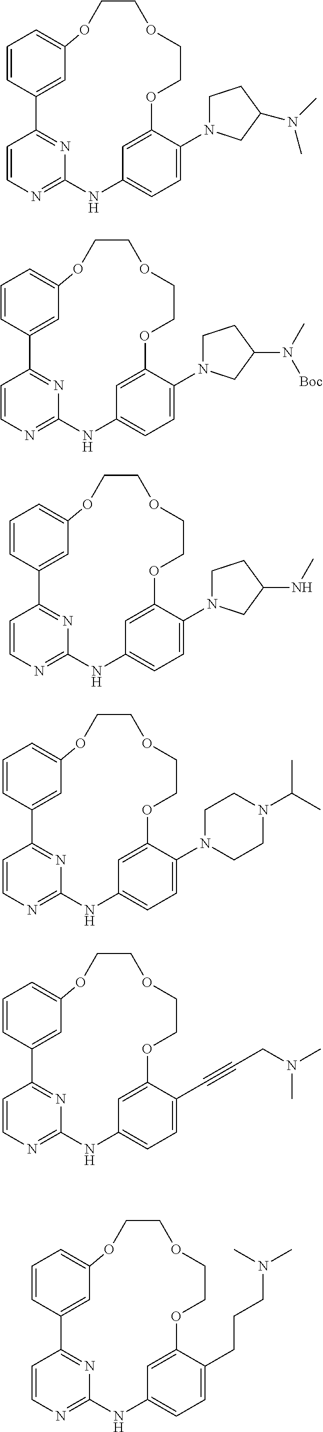 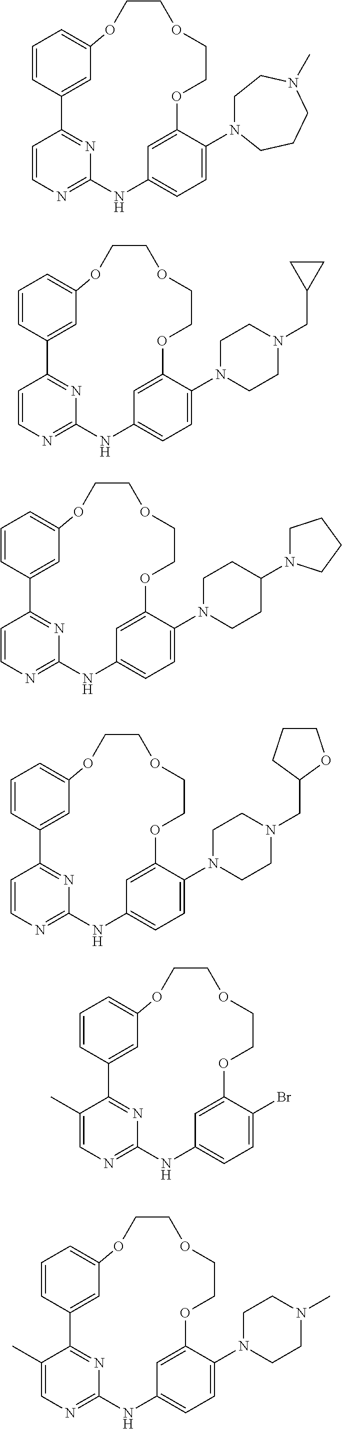 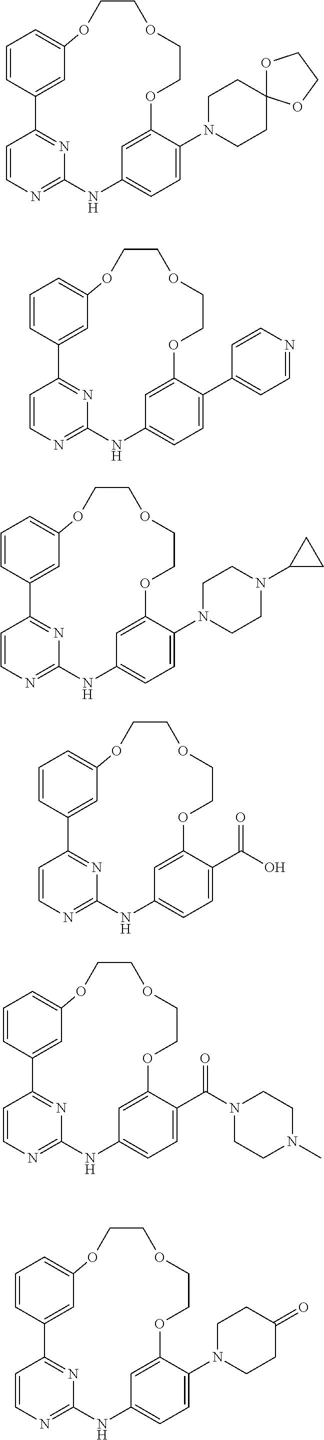 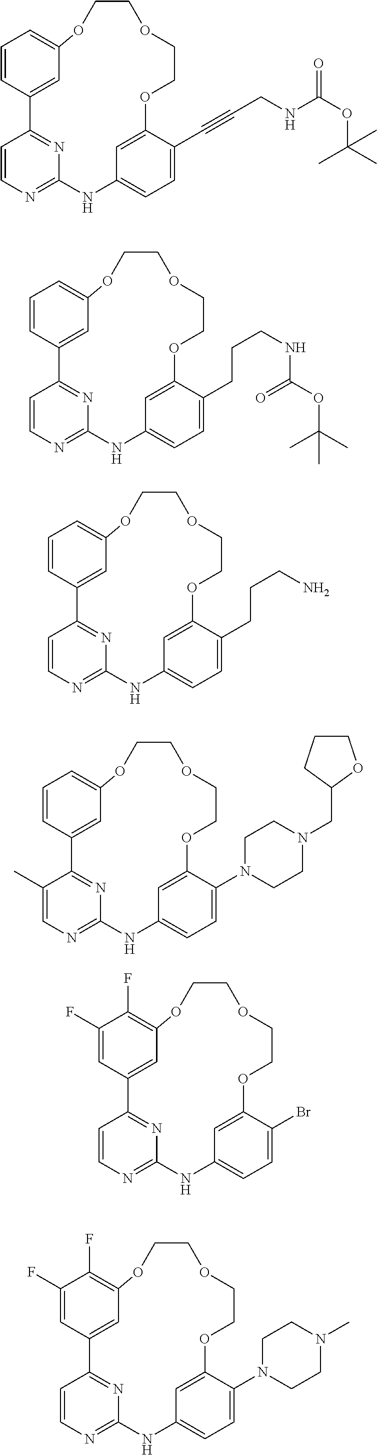 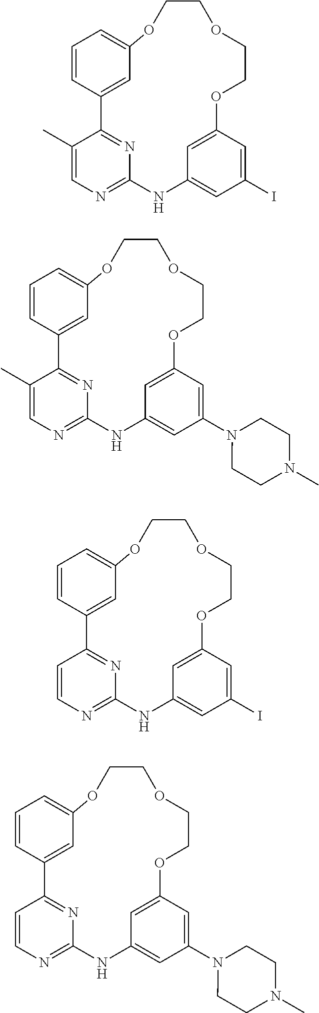
and the pharmaceutically acceptable salts and/or solvates thereof.

26. A pharmaceutical composition comprising at least one compound according to claim 18 and at least one pharmaceutically acceptable excipient.

27. A method for preparing a compound of formula (I) according to claim 18 comprising the coupling reaction between:
a compound of the following formula (II): 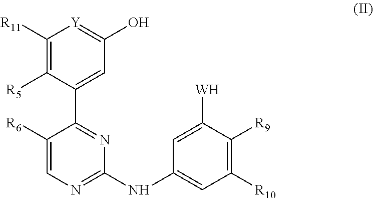
wherein W, Y, R₅, R₆, R₉, R₁₀ and R₁₁ are as defined in claim 1, and
a compound of the following formula (III): 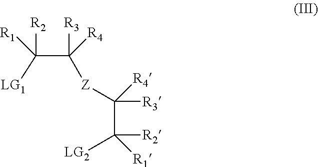
wherein Z, R₁, R₂, R₃, R₄, R₁′, R₂′, R₃′ and R₄′ are as defined in claim 1, and LG₁ and LG₂ each represent, independently of each other, a leaving group.

28. A method for preparing a compound according to claim 18 comprising the cyclization reaction of a compound of the following formula (VIa) or (VIb): 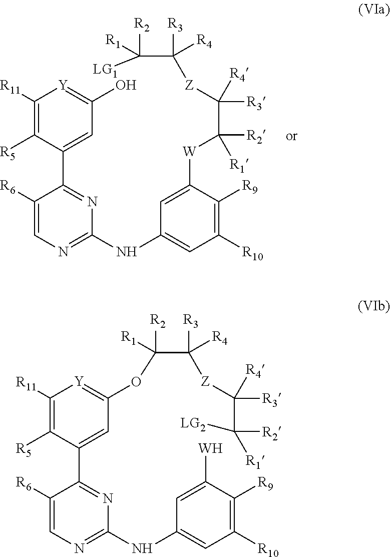
wherein W, Y, Z, R₁ to R₆, R₉ to R₁₁ and R₁′ to R₄′ are as defined in claim 1, and LG₁ and LG₂ each represent, independently of each other, a leaving group.

29. A method for preparing a compound according to claim 18, wherein R₉ and/or R₁₀ represents an optionally substituted (C₁-C₆)alkoxy, optionally substituted (C₁-C₆)thioalkoxy or NR₁₄R₁₅ group or an optionally substituted heterocycle comprising a heteroatom directly attached to the phenyl ring, comprising the coupling between a compound of the following formula (IVa) or (IVb): 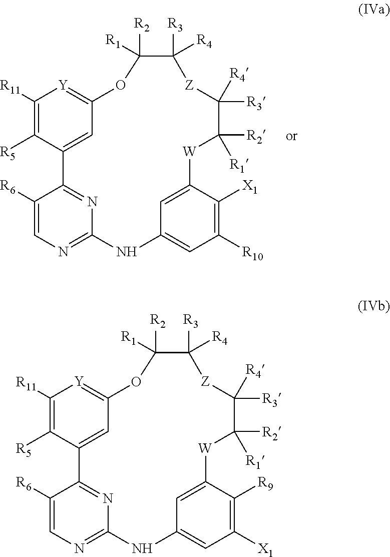
wherein W, Y, Z, R₁ to R₆, R₉ to R₁₁ and R₁′ to R₄′ are as defined in claim 1 and X₁ represents a halogen atom,
and respectively a compound of formula R₉H or R₁₀H wherein R₉ and R₁₀ are as defined above.

30. A method for preparing a compound according to claim 18, wherein R₉ and/or R₁₀ represents an optionally substituted (C₁-C₆)alkyl, optionally substituted (C₂-C₆)alkenyl or optionally substituted (C₂-C₆)alkynyl group, an optionally substituted carbocycle or an optionally substituted heterocycle attached to the phenyl ring by means of a carbon atom, comprising the coupling between a compound of the following formula (Va) or (Vb): 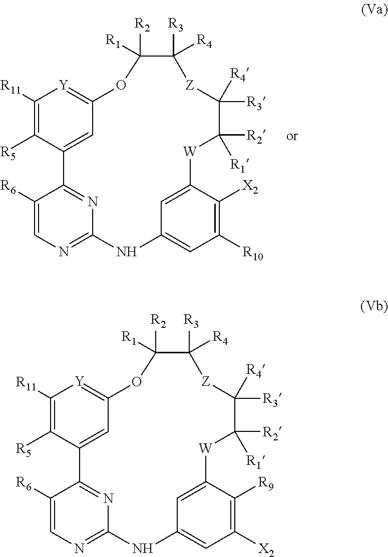
wherein W, Y, Z, R₁ to R₆, R₉ to R₁₁ and R₁′ to R₄′ are as defined in claim 1 and X₂ represents Br, Cl, I or OTf (OSO₂CF₃),
and respectively a compound of formula R₉—BR₅₂R₅₃ or R₁₀—BR₅₂R₅₃ wherein R₉ and R₁₀ are as defined above and R₅₂ and R₅₃ represent, independently of each other, an OH, (C₁-C₆)alkyl or (C₁-C₆)alkoxy group or R₅₂ and R₅₃ together form an —X₃— or —O—X₃—O— chain wherein X₃ represents a divalent hydrocarbon group comprising 2 to 15 carbon atoms.

31. The method for preparing a compound according to claim 24, wherein R₉ and/or R₁₀ represents —Z₁—(CH₂)ₘ—R₄₉ wherein Z₁ represents CH₂—CH₂, CH═CH or C≡C, comprising the following steps:
(1) Sonogashira coupling between a compound of formula (Va) or (Vb) as defined in claim 15, and a compound of formula HC≡C—(CH₂)ₘ—R₁₉ wherein m and R₄₉ are as defined in claim 7, to give a compound of formula (I) wherein R₉ or R₁₀ represents —C≡C—(CH₂)ₘ—R₄₉, and
(2) optionally reduction of the alkyne function of the compound of formula (I) obtained in the preceding step to give a compound of formula (I) wherein R₉ or R₁₀ represents —CH═CH—(CH₂)ₘ—R₄₉ or —(CH₂)ₘ₊₂—R₄₉.

32. A method for preparing a compound according to claim 18, wherein R₉ and/or R₁₀ represents a CO₂R₅₄ or CONR₅₅R₅₆ group, which comprises at least one of the following steps:
(a) to obtain a compound of formula (I) wherein R₉ and/or R₁₀ represents a CO₂H group, the reaction of a compound of formula (I) wherein R₉ and/or R₁₀ represents a halogen atom with CO₂;
(b) to obtain a compound of formula (I) wherein R₉ and/or R₁₀ represents a CO₂R₅₄ group with R₅₄≠H, the substitution reaction of a compound of formula (I) wherein R₉ and/or R₁₀ represents a CO₂H group, optionally obtained according to step (a), optionally in an activated form, with an alcohol of formula R₅₄OH;
(c) to obtain a compound of formula (I) wherein R₉ and/or R₁₀ represents a CONR₅₅R₅₆ group, the substitution reaction of a compound of formula (I) wherein R₉ and/or R₁₀ represents a CO₂H group, optionally obtained according to step (a), optionally in an activated form, with an amine of formula HNR₅₅R₅₆.

33. The compound according to claim 23, wherein n represents an integer equal to 2 or 3.

34. The compound according to claim 24, wherein R₉ and R₁₀ represent, independently of each other:
a hydrogen or halogen atom,
a CO₂R₅₄ group wherein R₅₄ represents a hydrogen atom,
a CONR₅₅R₅₆ group wherein R₅₅ and R₅₆ form with the nitrogen atom that bears them a saturated nitrogen containing heterocycle having 5, 6 or 7 members, comprising 1 or 2 heteroatoms selected from N and O of which at least one is a nitrogen atom, optionally substituted by one or more substituents selected from a halogen atom, (C₁-C₆)alkyl, oxo (═O), OR₂₀, NR₂₂R₂₃, CO₂R₂₅ and C(O)NR₃₁R₃₂,
a —Z₁—(CH₂)ₘ—R₄₉ group wherein Z₁ represents a single bond, CH₂—CH₂, C≡C, O or NR₅₀; m represents an integer between 1 and 4; R₅₀ represents a hydrogen atom or a (C₁-C₆)alkyl group; and R₄₉ represents OR₂₀, NR₂₂R₂₃, CO₂R₂₅, C(O)NR₃₁R₃₂, NR₃₃CO₂R₃₄, OC(O)NR₃₅R₃₆, NR₃₇CONR₃₈R₃₉ or OCO₂R₄₀, or
a saturated or aromatic heterocycle having 6 or 7 members, comprising 1 or 2 heteroatoms selected from N and O and comprising at least one nitrogen atom, optionally substituted by one or more substituents selected from:
a halogen atom,
a (C₁-C₆)alkyl group optionally substituted by one or more groups selected from a halogen atom, OR₁₆, NR₁₈R₁₉, a C₃ to C₆ monocyclic carbocycle and a 3- to 6-membered monocyclic heterocycle comprising 1 or 2 heteroatoms selected from N and O,
oxo (═O), OR₂₀, NR₂₂R₂₃, CO₂R₂₅, C(O)NR₃₁R₃₂, NR₃₃CO₂R₃₄, OC(O)NR₃₅R₃₆, NR₃₇CONR₃₈R₃₉ or OCO₂R₄₀ groups,
a C₃ to C₆ carbocycle optionally substituted by one or more groups selected from a halogen atom, a (C₁-C₆)alkyl group, oxo (═O), OR₄₁ and NR₄₃R₄₄,
a saturated 3- to 6-membered heterocycle comprising 1 or 2 heteroatoms selected from N and O, optionally substituted by one or more groups selected from a halogen atom, a (C₁-C₆)alkyl group, oxo (═O), OR₄₅ and NR₄₇R₄₈, and
an —O(CH₂)ₙO— group wherein n represents an integer equal to 2 or 3,
wherein:
R₁₆, R₁₈ to R₂₀, R₂₂ to R₂₆, R₂₉ to R₄₀, R₄₅ and R₄₇ to R₄₈ represent, independently of each other, a hydrogen atom or a (C₁-C₆)alkyl group, or
R₂₂ and R₂₃, R₃₁ and R₃₂, R₃₅ and R₃₆, R₃₈ and R₃₉, R₄₃ and R₄₄, and/or R₄₇ and R₄₈ together form, with the nitrogen atom that bears them, a heterocycle selected from piperazine, piperidine, morpholine and pyrrolidine, the heterocycle being optionally substituted by one or more groups selected from a halogen atom, a (C₁-C₆)alkyl group, and oxo (═O).

35. The compound according to claim 34, wherein R₄₉ represents NR₂₂R₂₃, NR₃₃CO₂R₃₄, or NR₃₇CONR₃₈R₃₉.

36. The method according to claim 28, wherein LG₁ and LG₂ each represent, independently of each other, a halogen atom.

37. A method for the treatment of cancer comprising the administration to a person in need thereof of an effective dose of a compound according to claim 18.
